(19) United States
(12) Patent Application Publication
(10) Pub. No.: US 2008/0064718 A1
(43) Pub. Date: Mar. 13, 2008

(54) INHIBITORS OF PROTEIN TYROSINE KINASE ACTIVITY

(76) Inventors: Oscar Mario Saavedra, Montreal (CA); Stephen William Claridge, Montreal (CA); Lijie Zhan, Montreal (CA); Franck Raeppel, Montreal (CA); Arkadii Vaisburg, Kirkland (CA); Stephane Raeppel, St. Lazare (CA); Frederic Gaudette, Verdun (CA); Ljubomir Isakovic, Beaconsfield (CA); Marie Claude Granger, Laprairie (CA); Jubrail Rahil, Dollard Des Ormeaux (CA); Michael Mannion, Montreal (CA); Robert Deziel, Mount-Royal (CA)

Correspondence Address: KEOWN & ZUCCHERO, LLP, 500 WEST CUMMINGS PARK, SUITE 1200, WOBURN, MA, 01801, US

(21) Appl. No.: 11/689,583

(22) Filed: Mar. 22, 2007

Related U.S. Application Data
(60) Provisional application No. 60/785,054, filed on Mar. 22, 2006.

(51) Int. Cl. A61K 31/4365; A61P 43/00; C07D 495/04

(52) U.S. Cl. 514/301; 546/114

(57) ABSTRACT

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling and HGF receptor signaling. The invention also provides compositions and methods for treating cell proliferative diseases and conditions.

### RELATED APPLICATIONS

This application claims the benefit of U.S. Provisional Patent Application Ser. No. 60/785,054, filed on Mar. 22, 2006, the contents of which are incorporated herein by reference in its entirety.

### BACKGROUND OF THE INVENTION

1. Field of the Invention

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling and HGF receptor signaling.

2. Summary of the Related Art

Tyrosine kinases may be classified as growth factor receptor (e.g. EGFR, PDGFR, FGFR and erbB2) or non-receptor (e.g. c-src and bcr-abi) kinases. The receptor type tyrosine kinases make up about 20 different subfamilies. The non-receptor type tyrosine kinases make up numerous subfamilies. These tyrosine kinases have diverse biological activity. Receptor tyrosine kinases are large enzymes that span the cell membrane and possess an extracellular binding domain for growth factors, a transmembrane domain, and an intracellular portion that functions as a kinase to phosphorylate a specific tyrosine residue in proteins and hence to influence cell proliferation. Aberrant or inappropriate protein kinase activity can contribute to the rise of disease states associated with such aberrant kinase activity.

Angiogenesis is an important component of certain normal physiological processes such as embryogenesis and wound healing, but aberrant angiogenesis contributes to some pathological disorders and in particular to tumor growth (Fan, T. P. D. et al., Trends Pharmacol. Sci. 1995, 16, 57-66; Folkman, J. Nat. Med. 1995, 1, 27-31). VEGF-A (vascular endothelial growth factor A) is a key factor promoting neovascularization (angiogenesis) of tumors (Jakeman, L. B. et al., Endocrinology 1993, 133, 848-859; Connolly, D. T. et al., J. Biol. Chem. 1989, 264, 20017-20024; Plowman, G. D. et al., Drug News Perspect. 1994, 7, 334-339; Straw, L. M. et al., Exp. Opin. Invest. Drugs 1998, 7, 553-573; and Shawver, L. K. et al., Drug Discov. Today 1997, 2, 50-63). VEGF induces endothelial cell proliferation and migration by signaling through two high affinity receptors, the fms-like tyrosine kinase receptor, Flt-1, and the kinase insert domain-containing receptor, KDR (De Vries, C. et al., Science 1992, 255, 989-991; Terman, B. I. et al., Biochem. Biophys. Res. Commun. 1992, 187, 1579-1586; Plate K. H. et al., Int. J. Cancer, 59: 520-529, 1994). These signaling responses are critically dependent upon receptor dimerization and activation of intrinsic receptor tyrosine kinase (RTK) activity. The binding of VEGF as a disulfide-linked homodimer stimulates receptor dimerization and activation of the RTK domain (Fuh G. et al., J. Biol. Chem., 273: 11197-11204, 1998). The kinase activity autophosphorylates cytoplasmic receptor tyrosine residues, which then serve as binding sites for molecules involved in the propagation of a signaling cascade. Although multiple pathways are likely to be elucidated for both receptors, KDR signaling is most extensively studied, with a mitogenic response suggested to involve ERK-1 and ERK-2 mitogen-activated protein kinases (Wheeler-Jones C. et al., FEBS Lett., 420: 28-32, 1997). Disruption of VEGF receptor signaling is a highly attractive therapeutic target in cancer, as angiogenesis is a prerequisite for all solid tumor growth, and that the mature endothelium remains relatively quiescent (with the exception of the female reproductive system and wound healing). A number of experimental approaches to inhibiting VEGF signaling have been examined, including use of neutralizing antibodies (Kim K. J. et al., Nature (Lond.), 362: 841-844, 1993; Kanai T. et al., Int. J. Cancer, 77: 933-936, 1998; Zhu Z. et al., Cancer Res., 58: 3209-3214, 1998), receptor antagonists (Siemeister G. et al., Proc. Natl. Acad. Sci. USA, 95: 4625-4629, 1998), soluble receptors (Lin P. et al., Cell Growth Differ., 9: 49-58, 1998), antisense constructs (Cheng S-Y. et al., Proc. Natl. Acad. Sci. USA, 93: 8502-8507, 1996) and dominant-negative strategies (Millauer B. et al., Cancer Res., 56: 1615-1620, 1996).

Despite the attractiveness of anti-angiogenic therapy by VEGF inhibition alone, several issues may limit this approach. VEGF expression levels can themselves be elevated by numerous diverse stimuli and perhaps most importantly, the hypoxic state of tumors resulting from VEGFr inhibition, can lead to the induction of factors that themselves promote tumor invasion and metastasis thus, potentially undermining the impact of VEGF inhibitors as cancer therapeutics (Pennacchietti S. et al., Cancer Cell. 2003 April; 3(4):347-61).

The HGF (hepatocyte growth factor) and the HGF receptor, c-met, are implicated in the ability of tumor cells to undermine the activity of VEGF inhibition (Pennacchietti S. et al., Cancer Cell. 2003 April; 3(4):347-61). HGF derived from either stromal fibroblasts surrounding tumor cells or expressed from the tumor itself has been suggested to play a critical role in tumor angiogenesis, invasion and metastasis (Camps J L et al., Proc Natl Acad Sci USA, 87: 75-9, 1990; and Nakamura T et al., Cancer Res, 57: 3305-13, 1997). For example, invasive growth of certain cancer cells is drastically enhanced by tumor-stromal interactions involving the HGF/c-Met (HGF receptor) pathway (Nishimura K et al., Int J Urol, 5: 276-81, 1998; Bae-Jump V et al., Gynecol Oncol, 73: 265-72, 1999; and Nakamura T et al., Biochem Biophys Res Commun, 122: 1450-9, 1984). HGF, which was originally identified as a potent mitogen for hepatocytes (Nakamura T et al., Nature, 342: 440-3, 1989; and Ebert M et al., Cancer Res, 54: 5775-8, 1994) is primarily secreted from stromal cells, and the secreted HGF can promote motility and invasion of various cancer cells that express c-Met in a paracrine manner (Di Renzo M F et al., Oncogene, 6: 1997-2003, 1991; Di Renzo M F et al., Cancer Res, 55: 1129-38, 1995; and Delehedde M et al., Eur J Biochem, 268: 4423-9, 2001). Binding of HGF to c-Met leads to receptor phosphorylation and activation of Ras/mitogen-activated protein kinase (MAPK) signaling pathway, thereby enhancing malignant behaviors of cancer cells (Delehedde M et al., Eur J Biochem, 268: 4423-9, 2001; and Bardelli A et al., Oncogene, 18: 1139-46, 1999). Moreover, stimulation of the HGF/c-met pathway itself can lead to the induction of VEGF expression, itself contributing directly to angiogenic activity (Saucier C et al., Proc Natl Acad Sci USA. 2004 Feb. 24; 101 (8):2345-50).

Thus, anti-tumor anti-angiogenic strategies or approaches that target both VEGF/VEGFr signaling and HGF/c-met signaling may circumvent the ability of tumor cells to overcome VEGF inhibition alone and may represent improved cancer therapeutics.

Here we describe small molecules that are potent inhibitors of protein tyrosine kinase activity, such as that of, for example, both the VEGF receptor KDR and the HGF receptor c-met.

### BRIEF SUMMARY OF THE INVENTION

The present invention provides new compounds and methods for treating cell proliferative diseases. The compounds of the invention are inhibitors of protein tyrosine kinase activity. Preferably, the compounds of the invention are dual function inhibitors, capable of inhibiting both VEGF and HGF receptor signaling. Accordingly, the invention provides new inhibitors of protein tyrosine kinase receptor signaling, such as for example, VEGF receptor signaling and HGF receptor signaling, including the VEGF receptor KDR and the HGF receptor c-met.

In a first aspect, the invention provides compounds of formula I that are useful as kinase inhibitors and, therefore, are useful research tools for the study of the role of kinases in both normal and disease states. Preferrably, the invention provides compounds of Formula I that are useful as inhibitors of VEGF receptor signaling and HGF receptor signaling and, therefore, are useful research tools for the study of the role of VEGF and HGF in both normal and disease states.

In a second aspect, the invention provides compounds of formula II that are useful as kinase inhibitors and, therefore, are useful research tools for the study of the role of kinases in both normal and disease states. Preferrably, the invention provides compounds of Formula II that are useful as inhibitors of VEGF receptor signaling and HGF receptor signaling and, therefore, are useful research tools for the study of the role of VEGF and HGF in both normal and disease states.

In a third aspect, the invention provides compounds of formula III that are useful as kinase inhibitors and, therefore, are useful research tools for the study of the role of kinases in both normal and disease states. Preferrably, the invention provides compounds of Formula III that are useful as inhibitors of VEGF receptor signaling and HGF receptor signaling and, therefore, are useful research tools for the study of the role of VEGF and HGF in both normal and disease states.

In a fourth aspect, the invention provides compounds of formula IV that are useful as kinase inhibitors and, therefore, are useful research tools for the study of the role of kinases in both normal and disease states. Preferrably, the invention provides compounds of Formula IV that are useful as inhibitors of VEGF receptor signaling and HGF receptor signaling and, therefore, are useful research tools for the study of the role of VEGF and HGF in both normal and disease states.

In a fifth aspect, the invention provides compounds of formula V that are useful as kinase inhibitors and, therefore, are useful research tools for the study of the role of kinases in both normal and disease states. Preferrably, the invention provides compounds of Formula V that are useful as inhibitors of VEGF receptor signaling and HGF receptor signaling and, therefore, are useful research tools for the study of the role of VEGF and HGF in both normal and disease states.

In a sixth aspect, the invention provides compounds of formula VI that are useful as kinase inhibitors and, therefore, are useful research tools for the study of the role of kinases in both normal and disease states. Preferrably, the invention provides compounds of Formula VI that are useful as inhibitors of VEGF receptor signaling and HGF receptor signaling and, therefore, are useful research tools for the study of the role of VEGF and HGF in both normal and disease states.

In a seventh aspect, the invention provides compounds of formula VII that are useful as kinase inhibitors and, therefore, are useful research tools for the study of the role of kinases in both normal and disease states. Preferrably, the invention provides compounds of Formula VII that are useful as inhibitors of VEGF receptor signaling and HGF receptor signaling and, therefore, are useful research tools for the study of the role of VEGF and HGF in both normal and disease states.

In an eighth aspect, the invention provides compounds of formula VIII that are useful as kinase inhibitors and, therefore, are useful research tools for the study of the role of kinases in both normal and disease states. Preferrably, the invention provides compounds of Formula VIII that are useful as inhibitors of VEGF receptor signaling and HGF receptor signaling and, therefore, are useful research tools for the study of the role of VEGF and HGF in both normal and disease states.

In a ninth aspect, the invention provides compounds of formula IX that are useful as kinase inhibitors and, therefore, are useful research tools for the study of the role of kinases in both normal and disease states. Preferrably, the invention provides compounds of Formula IX that are useful as inhibitors of VEGF receptor signaling and HGF receptor signaling and, therefore, are useful research tools for the study of the role of VEGF and HGF in both normal and disease states.

In a tenth aspect, the invention provides compounds of formula X that are useful as kinase inhibitors and, therefore, are useful research tools for the study of the role of kinases in both normal and disease states. Preferrably, the invention provides compounds of Formula X that are useful as inhibitors of VEGF receptor signaling and HGF receptor signaling and, therefore, are useful research tools for the study of the role of VEGF and HGF in both normal and disease states.

In an eleventh aspect, the invention provides compounds of formula XI that are useful as kinase inhibitors and, therefore, are useful research tools for the study of the role of kinases in both normal and disease states. Preferrably, the invention provides compounds of Formula XI that are useful as inhibitors of VEGF receptor signaling and HGF receptor signaling and, therefore, are useful research tools for the study of the role of VEGF and HGF in both normal and disease states.

In a twelfth aspect, the invention provides compounds of formula XII that are useful as kinase inhibitors and, therefore, are useful research tools for the study of the role of kinases in both normal and disease states. Preferrably, the invention provides compounds of Formula XII that are useful as inhibitors of VEGF receptor signaling and HGF receptor signaling and, therefore, are useful research tools for the study of the role of VEGF and HGF in both normal and disease states.

In a thirteenth aspect, the invention provides compounds of formula XIII that are useful as kinase inhibitors and, therefore, are useful research tools for the study of the role of kinases in both normal and disease states. Preferrably, the invention provides compounds of Formula XIII that are useful as inhibitors of VEGF receptor signaling and HGF receptor signaling and, therefore, are useful research tools for the study of the role of VEGF and HGF in both normal and disease states.

In a fourteenth aspect, the invention provides compounds of formula XIV that are useful as kinase inhibitors and, therefore, are useful research tools for the study of the role of kinases in both normal and disease states. Preferrably, the invention provides compounds of Formula XIV that are useful as inhibitors of VEGF receptor signaling and HGF receptor signaling and, therefore, are useful research tools for the study of the role of VEGF and HGF in both normal and disease states.

In a fifteenth aspect, the invention provides compounds of formula XV that are useful as kinase inhibitors and, therefore, are useful research tools for the study of the role of kinases in both normal and disease states. Preferrably, the invention provides compounds of Formula XV that are useful as inhibitors of VEGF receptor signaling and HGF receptor signaling and, therefore, are useful research tools for the study of the role of VEGF and HGF in both normal and disease states.

In a sixteenth aspect, the invention provides compounds of formula XVI that are useful as kinase inhibitors and, therefore, are useful research tools for the study of the role of kinases in both normal and disease states. Preferrably, the invention provides compounds of Formula XVI that are useful as inhibitors of VEGF receptor signaling and HGF receptor signaling and, therefore, are useful research tools for the study of the role of VEGF and HGF in both normal and disease states.

In a seventeenth aspect, the invention provides compositions comprising a compound that is an inhibitor of a protein tyrosine kinase, or a pharmaceutically acceptable salt thereof, and a pharmaceutically acceptable carrier, excipient or diluent. Preferably, the invention provides compositions comprising a compound that is an inhibitor of VEGF receptor signaling and HGF receptor signaling, or a pharmaceutically acceptable salt thereof, and a pharmaceutically acceptable carrier, excipient, or diluent.

The eighteenth aspect of the invention provides a method of inhibiting a protein tyrosine kinase, the method comprising contacting the kinase with a compound according to the present invention, or with a composition according to the present invention. Preferably the invention provides a method of inhibiting VEGF receptor signaling and HGF receptor signaling, the method comprising contacting the receptor with a compound according to the present invention, or with a composition according to the present invention. Inhibition of receptor protein kinase activity, preferably VEGF and HGF receptor signaling, can be in a cell or a multicellular organism. If in a multicellular organism, the method according to this aspect of the invention comprises administering to the organism a compound according to the present invention, or a composition according to the present invention. Preferably the organism is a mammal, more preferably a human.

The foregoing merely summarizes certain aspects of the invention and is not intended to be limiting in nature. These aspects and other aspects and embodiments are described more fully below.

### DETAILED DESCRIPTION OF THE PREFERRED EMBODIMENTS

The invention provides compounds and methods for inhibiting protein tyrosine kinase, preferably the VEGF receptor KDR and the HGF receptor c-met. The invention also provides compositions and methods for treating cell proliferative diseases and conditions. The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

For purposes of the present invention, the following definitions will be used (unless expressly stated otherwise):

The terms “inhibitor of VEGF receptor signaling” and “inhibitor of HGF receptor signaling” are used to identify a compound having a structure as defined herein, which is capable, respectively, of interacting with a VEGF receptor and a HGF receptor and inhibiting the activity of VEGF and HGF. In some preferred embodiments, such reduction of activity is at least about 50%, more preferably at least about 75%, and still more preferably at least about 90%.

Reference to “a compound of the formula (I), formula (II), etc.,” (or equivalently, “a compound according to the first aspect”, or “a compound of the present invention”, and the like), herein is understood to include reference to N-oxides, hydrates, solvates, pharmaceutically acceptable salts, prodrugs and complexes thereof, and racemic mixtures, diastereomers, enantiomers and tautomers thereof unless otherwise indicated.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperazinyl and piperidinyl (C₆); C₆-heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃ hydrocarbyl” includes a covalent bond, methyl, ethyl, ethenyl, ethynyl, propyl, propenyl, propynyl, and cyclopropyl.

The term “alkyl” is intended to mean a straight chain or branched aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” is intended to mean a saturated or unsaturated mono-, bi-, tri- or poly-cyclic hydrocarbon group having about 3 to 15 carbons, preferably having 3 to 12 carbons, preferably 3 to 8 carbons, more preferably 3 to 6 carbons, and more preferably still 5 or 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a heteroatom selected from the group consisting of O, S, and N.

The term “aryl” is intended to mean a mono-, bi-, tri- or polycyclic aromatic moiety, preferably a C₆-C₁₄aromatic moiety, preferably comprising one to three aromatic rings. Preferably, the aryl group is a C₆-C₁₀aryl group, more preferably a C₆aryl group. Preferred aryl groups include, without limitation, phenyl, naphthyl, anthracenyl, and fluorenyl.

The terms “aralkyl” or “arylalkyl” is intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic, in which case the group is also known as a heterocycloalkyl. In certain preferred embodiments, the heterocyclic group is a bridged heterocyclic group (for example, a bicyclic moiety with a methylene, ethylene or propylene bridge). In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” is intended to mean a mono-, bi-, tri- or polycyclic group having 5 to 14 ring atoms, preferably 5, 6, 9, or 10 ring atoms; having 6, 10, or 14 pi electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Preferred heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as fuor[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, independently selected non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkyamino, C₁-C₈alkoxycarbonyl, aryloxycarbonyl, C₂-C₈acyl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³), —NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkynyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O₀₋₂—, —SO₂N(R³¹)—, —N(R³¹)—SO₂— and —N(R³⁰)—SO₂N(R³¹)—.

A moiety that is substituted is one in which one or more (preferably one to four, preferably from one to three and more preferably one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a hydrocarbyl, heteroalkyl, heterocyclic and/or aryl group is unsubstituted.

In other preferred embodiments, a hydrocarbyl, heteroalkyl, heterocyclic and/or aryl group is substituted with from 1 to 3 independently selected substituents.

Preferred substituents on alkyl groups include, but are not limited to, hydroxyl, halogen (e.g., a single halogen substituent or multiple halo substituents; in the latter case, groups such as CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, —S(═O)₂R^{e}, —P(═O)₂R^{e}, S(═O)₂OR^{e}, —P(═O)₂OR^{e}, —NR^{b}R^{c}, —NR^{b}S(═O)₂R^{e}, —NR^{b}P(═O)₂R^{e}, —S(═O)₂NR^{b}R^{c}, —P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited about as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cylcoalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cylic substituents at any available point or points of attachement, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In certain preferred embodiments, a heterocyclic group is substituted on carbon, nitrogen and/or sulfur at one or more positions. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on ring groups, such as aryl, heteroaryl, cycloalkyl and heterocyclyl, include halogen, alkoxy and alkyl.

Especially preferred substituents on alkyl groups include halogen and hydroxy.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from one of the specified groups or the substituents being chosen from two or more of the specified groups. In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

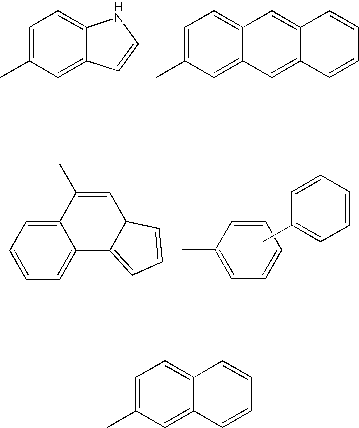

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, “unsubstituted aryl” does not include phenyl substituted with any of the optional substituents for which the definition of the moiety (above) otherwise provides.

A saturated or unsaturated three- to eight-membered carbocyclic ring is preferably a four- to seven-membered, more preferably five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated three- to eight-membered heterocyclic ring contains at least one heteroatom selected from oxygen, nitrogen, and sulfur atoms. The saturated or unsaturated three- to eight-membered heterocyclic ring preferably contains one or two heteroatoms with the remaining ring-constituting atoms being carbon atoms. The saturated or unsaturated three- to eight-membered heterocyclic ring is preferably a saturated or unsaturated four- to seven-membered heterocyclic ring, more preferably a saturated or unsaturated five- or six-membered heterocyclic ring. Examples of saturated or unsaturated three- to eight-membered heterocyclic groups include thienyl, pyridyl, 1,2,3-triazolyl, imidazolyl, isoxazolyl, pyrazolyl, piperazinyl, piperazino, piperidyl, piperidino, morpholinyl, morpholino, homopiperazinyl, homopiperazino, thiomorpholinyl, thiomorpholino, tetrahydropyrrolyl, and azepanyl.

A saturated or unsaturated carboxylic and heterocyclic group may condense with another saturated or heterocyclic group to form a bicyclic group, preferably a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁₋₆ alkyl groups, the two alkyl groups may combine together to form an alkylene chain, preferably a C₁₋₃ alkylene chain. Carbocyclic or heterocyclic groups having this crosslinked structure include bicyclo[2.2.2]octanyl and norbornanyl.

The terms “kinase inhibitor” and “inhibitor of kinase activity”, and the like, are used to identify a compound which is capable of interacting with a kinase and inhibiting its enzymatic activity.

The term “inhibiting kinase enzymatic activity” is used to mean reducing the ability of a kinase to transfer a phosphate group from a donor molecule, such as ATP, to a specific target molecule (substrate). For example, the inhibition of kinase activity may be at least about 10%. In some preferred embodiments of the invention, such reduction of kinase activity is at least about 50%, more preferably at least about 75%, and still more preferably at least about 90%. In other preferred embodiments, kinase activity is reduced by at least 95% and even more preferably by at least 99%. The IC₅₀ value is the concentration of kinase inhibitor which reduces the activity of a kinase to 50% of the uninhibited enzyme.

The term “inhibiting effective amount” is meant to denote a dosage sufficient to cause inhibition of kinase activity. The kinase may be in a cell, which in turn may be in a multicellular organism. The multicellular organism may be, for example, a plant, a fungus or an animal, preferably a mammal and more preferably a human. The fungus may be infecting a plant or a mammal, preferably a human, and could therefore be located in and/or on the plant or mammal. If the kinase is in a multicellular organism, the method according to this aspect of the invention comprises the step of administering to the organism a compound or composition according to the present invention. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

Preferably, such inhibition is specific, i.e., the kinase inhibitor reduces the ability of a kinase to transfer a phosphate group from a donor molecule, such as ATP, to a specific target molecule (substrate) at a concentration that is lower than the concentration of the inhibitor that is required to produce another, unrelated biological effect. Preferably, the concentration of the inhibitor required for kinase inhibitory activity is at least 2-fold lower, more preferably at least 5-fold lower, even more preferably at least 10-fold lower, and most preferably at least 20-fold lower than the concentration required to produce an unrelated biological effect.

The term “therapeutically effective amount” as employed herein is an amount of a compound of the invention, that when administered to a patient, treats the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

The term “patient” as employed herein for the purposes of the present invention includes humans and other animals, particularly mammals, and other organisms. Thus the compounds, compositions and methods of the present invention are applicable to both human therapy and veterinary applications. In a preferred embodiment the patient is a mammal, and in a most preferred embodiment the patient is human.

The terms “treating”, “treatment”, or the like, as used herein covers the treatment of a disease-state in an animal and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, preferably eliminating or curing of the disease. In a preferred embodiment of the present invention the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The present invention also includes prodrugs of compounds of the invention. The term “prodrug” is intended to represent covalently bonded carriers, which are capable of releasing the active ingredient of the prodrug when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the present invention include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of the invention, amides (e.g., trifluoroacetylamino, acetylamino, and the like), and the like.

The compounds of the invention may be administered in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₁₆-alkoxycarbonyloxyethyl esters (e.g., 1-methoxycarbonyloxyethyl) and may be formed at any carboxy group in the compounds of this invention

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁-C₆alkyl or N,N-di-C₁-C₆alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention, or a salt and/or solvate thereof. Solvates of the compounds of the present invention include, for example, hydrates.

Another aspect of the invention provides compositions including a compound, N-oxide, hydrate, solvate, pharmaceutically acceptable salt, complex or prodrug of a compound according to the present invention as described herein, or a racemic mixture, diastereomer, enantiomer or tautomer thereof. For example, in one embodiment of the invention, a composition comprises a compound, N-oxide, hydrate, solvate, pharmaceutically acceptable salt, complex or prodrug of a compound according to the present invention as described herein present in at least about 30% enantiomeric or diastereomeric excess. In certain desirable embodiments of the invention, the compound, N-oxide, hydrates, solvate, pharmaceutically acceptable salt, complex or prodrug is present in at least about 50%, at least about 80%, or even at least about 90% enantiomeric or diastereomeric excess. In certain other desirable embodiments of the invention, the compound, N-oxide, hydrate, solvate, pharmaceutically acceptable salt, complex or prodrug is present in at least about 95%, more preferably at least about 98% and even more preferably at least about 99% enantiomeric or diastereomeric excess. In other embodiments of the invention, a compound, N-oxide, hydrate, solvate, pharmaceutically acceptable salt, complex or prodrug is present as a substantially racemic mixture.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of formula (I) in at least about 30% diastereomeric or enantiomeric excess. In certain embodiments of the invention, the compound is present in at least about 50% enantiomeric or diastereomeric excess, in at least about 80% enantiomeric or diastereomeric excess, or even in at least about 90% enantiomeric or diastereomeric excess. In certain more preferred embodiments of the invention, the compound is present in at least about 95%, even more preferably in at least about 98% enantiomeric or diastereomeric excess, and most preferably in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

Throughout the specification, preferred embodiments of one or more chemical substituents are identified. Also preferred are combinations of preferred embodiments. For example, the invention describes preferred embodiments of R⁷ in the compounds and describes preferred embodiments of group W. Thus, as an example, also contemplated as within the scope of the invention are compounds in which preferred examples of R⁷ are as described and in which preferred examples of group W are as described. Furthermore, compounds excluded from any one particular genus of compounds (e.g., through a proviso clause) are intended to be excluded from the scope of the invention entirely, including from other disclosed genera, unless expressly stated to the contrary.

### Compounds

According to one embodiment, the invention provides compounds of Formula (I) and racemic mixtures, diastereomers and enantiomers thereof: 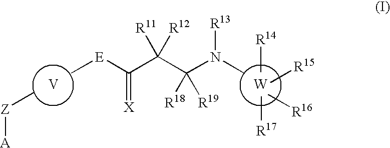
and N-oxides, hydrates, solvates, pharmaceutically acceptable salts, prodrugs and complexes thereof, wherein,
A is a structure selected from the group consisting of 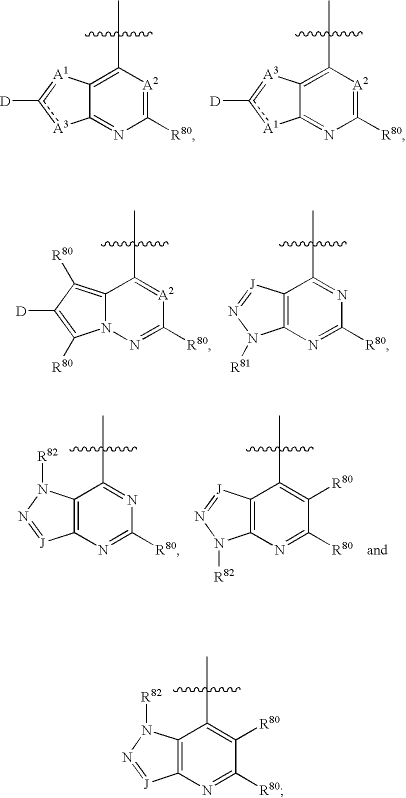
wherein
A¹ is selected from the group consisting of —CH₂—, —O—, —S—, —N(H)—, —N(C₁-C₆ alkyl)-, —N—(Y-aryl)-, —N—OMe, —NCH₂OMe and N-Bn;
Y is a bond or —(C(R^{x})(H))ₜ—, wherein t is an integer from 1 to 6; and
R^{x} at each occurrence is independently selected from the group consisting of H and C₁-C₆ alkyl, wherein the C₁-C₆ alkyl is optionally substituted;
A² is selected from the group consisting of N and CR, wherein R is selected from the group consisting of —H, halogen, —CN, C₁-C₆ alkyl, C₂-C₆ alkenyl, C₂-C₆ alkynyl, —COOH and —C(O)Oalkyl, wherein the C₁-C₆ alkyl, C₂-C₆ alkenyl, C₂-C₆ alkynyl and —C(O)Oalkyl are optionally substituted;
A³ is selected from the group consisting of C-D and N;
each R⁸⁰ is independently selected from the group consisting of H, halogen, NO₂, cyano, OR⁸³, N(R⁸³)₂, CO₂R⁸³, C(O)N(R⁸³)₂, SO₂R⁸³, SO₂N(R⁸³)₂, NR⁸³SO₂R⁸³, NR⁸³C(O)R⁸³, NR⁸³CO₂R⁸³, —CO(CH₂)₁R⁸³, —CONH(CH₂)₁R⁸³, alkylaminoalkyl, alkylaminoalkynyl, C₁-C₆alkyl, substituted C₁-C₆alkyl, C₃-C₇cycloalkyl, substituted C₃-C₇cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl, and substituted heterocycloalkyl;
J is CR⁸⁰ or N;
R⁸¹ is selected from the group consisting of H, C₁-C₆alkyl or substituted C₁-C₆alkyl;
R⁸³ is selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl, and substituted heterocycloalkyl; or
two R⁸³ taken together with the N atom to which they are attached form a heterocyclic ring; and
D is selected from the group consisting of R⁷, R¹ and R²¹, wherein
R⁷ is selected from the group consisting of —H, halogen, nitro, azido, C₁-C₆ alkyl, C₃-C₁₀ cycloalkyl, —C(O)NR⁴²R⁴³, —C(O)N(R¹³)NR⁴²R⁴³, —C(S)NR⁴²R⁴³, —C(S)N(R¹³)NR⁴²R⁴³, —Y—NR⁴²R⁴³, —NR⁴²C(═O)R⁴³, —SO₂R⁴², —SO₂NR⁴²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, —C(═NR⁴²)N³⁷R⁴³, —NR³⁷C(═NR⁴²)NR³⁷R⁴³, —C(O)R⁴², —C(S)R⁴², —CO₂R⁴², —C(O)(heterocyclyl), —C(O)(C₆-C₁₀ aryl), —C(O)(heteroaryl), —Y—(C₆-C₁₀ aryl), —Y-(heteroaryl), —Y-(5-10 membered heterocyclyl), —NR^{6a}R^{6b}, —NR^{6a}SO₂R^{6b}, —NR^{6a}C(O)R^{6b}, —OC(O)R^{6a}, —NR^{6a}C(O)OR^{6b}, —OC(O)NR^{6a}R^{6b}, —OR^{6a}, SR^{6a}, S(O)R^{6a}, —SO₂R^{6a}, —SO₃R^{6a}, —SO₂NR^{6a}R^{6b}, —SO₂NR⁴²R⁴³, —C(O)R^{6a}, —C(S)R^{6a}, —CO₂R^{6a}, —CONR^{6a}R^{6b}, —(C₁-C₄)fluoroalkyl, —(C₁-C₄)fluoroalkoxy, —(CZ³Z⁴)ₐCN, wherein a is an integer ranging from 0 to 6, and the aforementioned R⁷ groups other than —H and halogen are optionally substituted, or R⁷ is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R⁴² and R⁴³ is independently selected from the group consisting of H, C₁-C₆ alkyl, —Y⁴—(C₃-C₁₀ cycloalkyl), —Y⁴—(C₆-C₁₀ aryl), —Y⁴—(C₆-C₁₀ heteroaryl), —Y⁴-(5-10 membered heterocyclyl), —Y⁴—O—Y¹—OR³⁷, —Y¹—CO₂—R³⁷, and —Y⁴—OR³⁷, wherein the alkyl, cycloalkyl, aryl, heteroaryl and heterocyclyl moieties of the foregoing R⁴² and R⁴³ groups are optionally substituted by 1 or more substituents independently selected from R⁴⁴; or
R⁴² and R⁴³ taken together with the nitrogen to which they are attached form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted by 1 to 5 R⁴⁴ substituents, with the proviso that R⁴² and R⁴³ are not both bonded to the nitrogen directly through an oxygen;
Y is a bond or —(C(R^{y})(H))ₜ—, wherein t is an integer from 1 to 6; and
R^{y} at each occurrence is independently selected from the group consisting of H and C₁-C₆ alkyl, wherein the C₁-C₆ alkyl is optionally substituted;
Y⁴ is a bond or is —(C(R³⁷)(H))ₙ, wherein n is an integer ranging from 1 to 6;
R³⁷ is selected from H, OR³⁶, C₁-C₆ alkyl and C₃-C₁₀ cycloalkyl;
Y¹ is —(C(R³⁷)(H))₁₋₆;
each R⁴⁴ is independently selected from the group consisting of halo, cyano, nitro, trifluoromethoxy, trifluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹, —OR³⁷, —SO₂NR³⁶R³⁹, —SO₂R³⁶, —NR³⁶SO₂R³⁹, —NR³⁶SO₂NR³⁷R⁴¹, C₁-C₆ alkyl, C₂-C₆ alkenyl, C₂-C₆ alkynyl, C₃-C₁₀ cycloalkyl, —C₁-C₆ alkylamino, —(CH₂)ⱼ—O—(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl), —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5 to 10 membered heterocyclyl), —C(O)(CH₂)ₙ(5 to 10 membered heterocyclyl), —(CH₂)ⱼNR³⁹(CH₂)NR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, —(CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR³⁹(CH₂)ₙR³⁶, —SO₂(CH₂)ₙ(C₆-C₁₀ aryl), and —SO₂(CH₂)ₙ(5 to 10 membered heterocyclyl) wherein, j is an integer from 0 to 2, n is an integer from 0 to 6 and i is an integer ranging from 2 to 6, the —(CH₂)ᵢ— and —(CH₂)ₙ₁— moieties of the foregoing R⁴⁴ groups optionally include a carbon-carbon double or triple bond wherein n is an integer from 2 to 6, and the alkyl, aryl and heterocyclyl moieties of the foregoing R⁴⁴ groups are optionally substituted by 1 or more substituents independently selected from the group consisting of halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, —SO₂R³⁶, —SO₂NR³⁶R³⁹, C₁-C₆ alkyl, C₃-C₁₀ cycloalkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷, wherein n is an integer from 0 to 6 and i is an integer from 2 to 6;
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, —(CH₂)ₙCN(CH₂)ₙOR³⁷, —(CH₂)ₙCN(CH₂)ₙR³⁷, and —(CH₂)ₙOR³⁷, wherein n is an integer ranging from 0 to 6 and i is an integer ranging from 2 to 6, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —N³⁷R⁴¹, —C₁-C₆ alkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer ranging from 0 to 6 and i is an integer ranging from 2 to 6, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6;
each R³⁷ and R⁴¹ is independently selected from H, OR³⁶, C₁-C₆ alkyl and C₃-C₁₀ cycloalkyl;
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶)ᵤ-(C₅-C₆)cycloalkenyl, —(CZ⁵Z⁶)ᵤ-aryl, —(CZ⁵Z⁶)ᵤ-heterocycle, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of halogen, cyano, nitro, tetrazolyl, guanidino, amidino, methylguanidino, azido, —C(O)Z⁷, —OC(O)NH₂, —OC(O) NHZ⁷, —OC(O)NZ⁷Z⁸, —NHC(O)Z⁷, —NHC(O)NH₂, —NHC(O)NHZ⁷, —NHC(O)NZ⁷Z⁸, C(O)OH, —C(O)OZ⁷, —C(O)NH₂, —C(O)NHZ⁷, —C(O)NZ⁷Z⁸, —P(O)₃H₂, —P(O)₃(Z⁷)₂, —S(O)₃H, —S(O)Z⁷, —S(O)₂Z⁷, —S(O)₃Z⁷, -Z⁷, —OZ⁷, —OH, —NH₂, —NHZ⁷, —NZ⁷Z⁸, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, —(CZ⁹Z¹⁰)ᵣNZ⁷Z⁸, —X⁶(CZ⁹Z¹¹)ᵣ-(C₃-C₈)cycloalkyl, —X⁶(CZ⁹Z¹¹)ᵣ-(C₅-C₈)cycloalkenyl, —X⁶(CZ⁹Z¹⁰)ᵣ-aryl and —X⁶(CZ⁹Z¹⁰)ᵣ-heterocycle, wherein
r is 1, 2, 3 or 4;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of an alkyl of 1 to 12 carbon atoms, an alkenyl of 2 to 12 carbon atoms, an alkynyl of 2 to 12 carbon atoms, a cycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, or
Z⁷ and Z⁸ together may optionally form a heterocycle;
Z⁹ and Z¹⁰ are independently selected from the group consisting of H, F, a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl];
R¹ is —C≡CH or —C≡C—(CR⁴⁵R⁴⁵)ₙ—R⁴⁶; n is an integer from 0 to 6;
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷, —(CH₂)ₙNR⁴⁷R⁴⁸, —(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;

R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙC(O)R⁴⁹, —C(O)₂R⁴⁹, (CH₂)ₙSR⁴⁹, —(CH₂)ₙS(O)R⁴⁹—(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring; and
R²¹ is the group defined by -(Z¹¹)-(Z2)ₘ-(Z¹³)ₘ₁, wherein
Z¹¹ is heterocyclyl, when m and ml are 0, or heterocyclylene, when either m or ml are 1,
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆) alkyl-NR⁵³R⁵³ (C₁-C₃)alkyl, (CH₂)_{q}OR⁵³—C(O)R⁵⁴ and —C(O)OR⁵³
q is 0, 1, 2, 3 or 4;
each R⁵³ is independently (C₁-C₃)alkyl;
R⁵⁴ is (C₁-C₃)alkyl or N(H)R⁵³;
R⁵⁶ is selected from the group consisting of NH₂, (C₁-C₃)alkyl and OR⁵²;
V is a 5 to 7 membered cycloalkyl, aryl, heterocylic or heteroaryl ring system, any of which is optionally substituted with 0 to 4 R² groups;
R² at each occurrence is independently selected from the group consisting of —H, halogen, trihalomethyl, —O-trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, alkylamino, —CH₂(CH₂)₀₋₄-T², wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
each R³ is independently selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
Z is selected from the group consisting of —O—, —S—, —CH₂—, NBn and —NR⁵—, wherein R⁵ is selected from the group consisting of H, C₁-C₆ alkyl, an optionally substituted (C₁-C₅)acyl and C₁-C₆ alkyl-O—C(O), wherein C₁-C₆ alkyl is optionally substituted;
E is selected from the group consisting of —N(H)—, —N(C₁-C₆alkyl)-, —CH₂N(H)— and —N(H)CH₂—;
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted;
each R¹³ is independently selected from the group consisting of H, C₁-C₆alkyl, substituted C₁-C₆alkyl, cycloalkyl, substituted cycloalkyl, OH, unsubstituted —O—(C₁-C₆alkyl), substituted —O—(C₁-C₆alkyl); or
R¹² and R¹³ taken together with the atoms to which they are attached optionally form a 4 to 8 membered cycloalkyl or heterocyclic ring system, which ring system is optionally substituted; or
R¹³ and R¹⁴ taken together with the atoms to which they are attached optionally form a 4 to 8 membered cycloalkyl or heterocyclic ring system, which ring system is optionally substituted;
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F;
X is selected from the group consisting of O, S, NH, N-alkyl, N—OH, N—O-alkyl, and NCN;
W is selected from the group consisting of H, alkyl, alkenyl, alkynyl, —(CH₂)₀₋₅(five- to ten-membered cycloalkyl), —(CH₂)₀₋₅(aryl), —(CH₂)₀₋₅(heterocylic) and —(CH₂)₀₋₅(heteroaryl), each of which is optionally substituted; and
R¹⁴R¹⁵, R¹⁶ and R¹⁷ are independently selected from the group consisting of —H, halogen, trihalomethyl, —O-trihalomethyl, —CN, —NO₂, —NH₂, —OR³, —OCF₃, —NR³R⁴, —S(O)₀₋₂R³, S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
with the proviso that Formula (I) excludes those compounds wherein
V is an unsubstituted 5 or 6 membered aryl ring system or an unsubstituted 5 or 6 membered heteroaryl ring system containing between one and three heteroatoms;
Z is selected from the group consisting of O, S, NH and N (optionally substituted C₁-C₄alkyl); and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, —NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of formula (I-A) and racemic mixtures, diastereomers and enantiomers thereof: 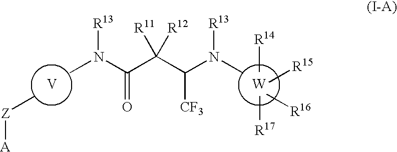
and N-oxides, hydrates, solvates, pharmaceutically acceptable salts, prodrugs and complexes thereof, wherein A, Z, V, E, W, R¹¹, R¹², R¹³, R¹⁴, R¹⁵, R¹⁶ and R¹⁷ are as defined in Formula (I).

In a preferred embodiment of the compounds according to Formula (I-A), W is phenyl.

According to another embodiment, the invention provides compounds of Formula (II) and racemic mixtures, diastereomers and enantiomers thereof: 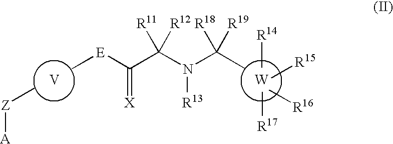
and N-oxides, hydrates, solvates, pharmaceutically acceptable salts, prodrugs and complexes thereof, wherein A, Z, V, E, X, W, R¹¹, R¹², R¹³, R¹⁴, R¹⁵, R¹⁶, R¹⁷, R¹⁸ and R¹⁹ are as defined in Formula (I);
with the proviso that Formula (II) excludes those compounds wherein
V is an unsubstituted 5 or 6 membered aryl ring system or an unsubstituted 5 or 6 membered heteroaryl ring system containing between one and three heteroatoms;
Z is selected from the group consisting of O, S, NH and N (optionally substituted C₁-C₄alkyl);
X is O; and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of formula (II-A) and racemic mixtures, diastereomers and enantiomers thereof: 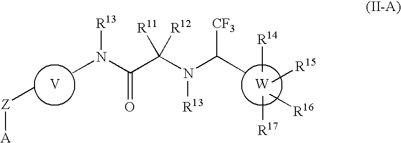
and N-oxides, hydrates, solvates, pharmaceutically acceptable salts, prodrugs and complexes thereof, wherein A, Z, V, E, W, R¹¹, R¹², R¹³, R¹⁴, R¹⁵, R¹⁶ and R¹⁷ are as defined in Formula (I).

In a preferred embodiment of the compounds according to Formula (II-A), W is phenyl.

According to another embodiment, the invention provides compounds of Formula (III) and racemic mixtures, diastereomers and enantiomers thereof: 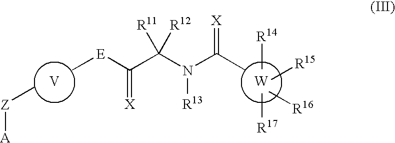
and N-oxides, hydrates, solvates, pharmaceutically acceptable salts, prodrugs and complexes thereof, wherein Z, V, E, X, W, R¹¹, R¹², R¹³, R¹⁴, R¹⁵, R¹⁶, R¹⁷, R¹⁸ and R¹⁹ are as defined in Formula (I); and
A is selected from the group consisting of 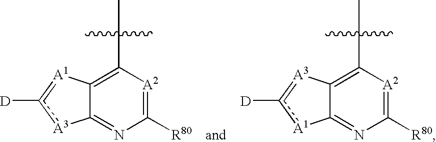
wherein
D, A1, A2, A3 and R⁸⁰ are as defined in Formula (I);
with the proviso that compounds of Formula (III) exclude those compounds wherein
A¹ and A² are as defined in Formula (I);
A³ is selected from the group consisting of C(R^{a}) and N; and
D and R^{a} are independently selected from the group consisting of H, halogen, NO₂, cyano, OR^{c}, NR^{c}R^{c}, CO₂R^{c}, C(O)NR^{c}R^{c}, SO₂R^{c}, SO₂NR^{c}R^{c}, NR^{c}SO₂R^{c}, NR^{c}C(O)R^{c}, NR^{c}CO₂R^{c}, —CO(CH₂)₀₋₄R^{c}, —CONH(CH₂)₀₋₄R^{c}, alkylaminoalkyl, alkylaminoalkynyl, C₁-C₆alkyl, substituted C₁-C₆alkyl, C₃-C₇cycloalkyl, substituted C₃-C₇cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl and substituted heterocycloalkyl; wherein
each R^{c} is independently selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl and substituted heterocycloalkyl; and
with the proviso that Formula (III) excludes those compounds wherein
V is an unsubstituted 5 or 6 membered aryl ring system or an unsubstituted 5 or 6 membered heteroaryl ring system containing between one and three heteroatoms;
Z is selected from the group consisting of O, S, NH and N (optionally substituted C₁-C₄alkyl);
X is O; and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

According to another embodiment, the invention provides compounds of Formula (IV) and racemic mixtures, diastereomers and enantiomers thereof: 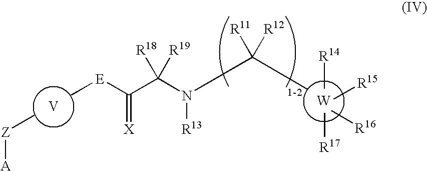
and N-oxides, hydrates, solvates, pharmaceutically acceptable salts, prodrugs and complexes thereof, wherein A, Z, V, E, X, W, R¹¹, R¹², R¹³, R¹⁴, R¹⁵, R¹⁶, R¹⁷, R¹⁸ and R¹⁹ are as defined in Formula (I);
with the proviso that Formula (IV) excludes those compounds wherein
V is an unsubstituted 5 or 6 membered aryl ring system or an unsubstituted 5 or 6 membered heteroaryl ring system containing between one and three heteroatoms;
Z is selected from the group consisting of O, S, NH and N (optionally substituted C₁-C₄alkyl);
X is O; and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of formula (IV-A) and racemic mixtures, diastereomers and enantiomers thereof: 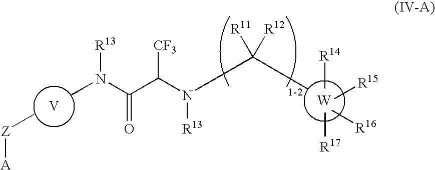
and N-oxides, hydrates, solvates, pharmaceutically acceptable salts, prodrugs and complexes thereof, wherein A, Z, V, W, R¹¹, R¹², R¹³, R¹⁴, R¹⁵, R¹⁶ and R¹⁷ are as defined in Formula (I).

In a preferred embodiment of the compounds according to Formula (IV-A), W is phenyl.

According to another embodiment, the invention provides compounds of Formula (V) and racemic mixtures, diastereomers and enantiomers thereof: 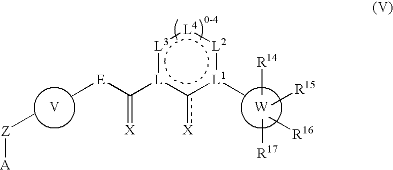
and N-oxides, hydrates, solvates, pharmaceutically acceptable salts, prodrugs and complexes thereof, wherein A, Z, V, E, X, W, R¹⁴, R¹⁵, R¹⁶ and R¹⁷ are as defined in Formula (I); [symbol]
is a single or double bond;
X¹ is selected from the group consisting of O, S, CH₂, N—CN, N—O-alkyl, NH and N(C₁-C₆alkyl) when [symbol] is a double bond, or
X¹ is selected from the group consisting of H, halogen, alkyl, alkenyl, alkynyl, CN, alkoxy, NH(alkyl) and alkyl-thio, each of which is optionally substituted, when [symbol] is a single bond;
L and L¹ are independently selected from the group consisting of —CH—, —N—, —C(halogen)- and —C(C₁-C₆alkyl)-;
L and L³ are independently selected from the group consisting of CH, CH₂, N, O and S;
L⁴ is selected from the group consisting of absent, CH, CH₂, N, O and S; and the group 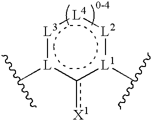
is aromatic or non-aromatic, provided that two 0 are not adjacent to each other; with the proviso that when [symbol] is a single bond, Formula (V) excludes those compounds wherein
Z is O;
V is a 6 membered aryl ring system or a 6 membered heteroaryl ring system containing one heteroatom; and
X¹ is selected from the group consisting of H, halogen, alkyl, alkenyl, alkynyl, CN and alkoxy;
provided that this proviso does not exclude those compounds wherein W is substituted by either an alkenyl or alkynyl.
with the proviso that Formula (V) excludes those compounds wherein
Z is selected from the group consisting of O, S, CH₂, N(Bn), N(H) and N (optionally substituted alkyl);
E is N(H) or N(alkyl);
X is O;
D is selected from the group consisting of H, halogen, NO₂, cyano, OR^{b}, NR^{b}R^{b}, CO₂R^{b}, C(O)NR^{b}R^{b}, SO₂R^{b}, SO₂NR^{b}R^{b}, NR^{b}SO₂R^{b}, NR^{b}C(O)R^{b}, NR^{b}CO₂R^{b}, —CO(CH₂)₁R^{b}, —CONH(CH₂)₁R^{b}, alkylaminoalkyl, alklaminoalkynyl, C₁-C₆alkyl, substituted C₁-C₆alkyl, C₃-C₇cycloalkyl, substituted C₃-C₇ cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl and substituted heterocycloalkyl;
   wherein R^{b} is selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl and substituted heterocycloalkyl; and the group 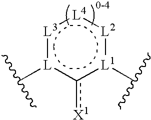
is selected from the group consisting of aryl, heteroaryl, and heterocylcoalkyl; and
with the proviso that Formula (V) excludes those compounds wherein
A is selected from the group consisting of 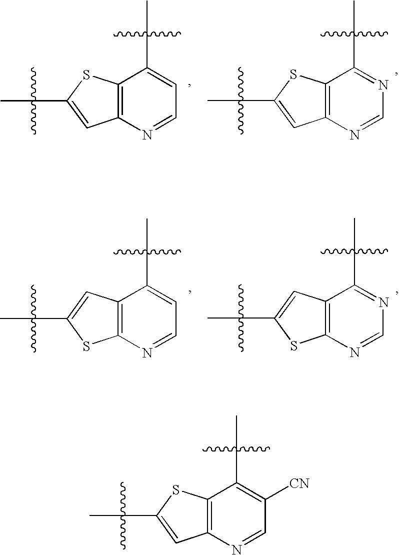
E is selected from the group consisting of —N(H)—, —N(C₁-C₆alkyl)- and —N(H)CH₂—;
R¹⁴, R¹⁵, R¹⁶ and R¹⁷ are each H;
Z is selected from the group consisting of —O—, —N(C(O)(C₁-C₆alkyl)), —S—, —CH₂—, —N(H)—, and —N(C₁-C₆alkyl); and
D is selected from the group consisting of —H, C₁-C₆ alkyl, C₃-C₁₀ cycloalkyl, —C(O)NR⁴²R⁴³, —Y—NR⁴²R⁴³, —NR⁴²C(═O)R⁴, —SO₂R⁴², —SO₂NR⁴²R⁴³, —NR³⁷SO₂R⁴², —NR³⁷S₂NR⁴²R⁴³, C(═N—OR⁴²)R⁴³, —C(═NR⁴²)R⁴³, —NR³⁷C(═NR⁴²)R⁴³, —C(═NR⁴²)N³⁷R⁴³, —NR³⁷C(═NR⁴²)NR³⁷R⁴³, —C(O)R⁴², —CO₂R⁴², —C(O)(C₆-C₁₀ aryl), —Y—(C₆-C₁₀ aryl), —Y-(5-10 membered heterocyclyl), —CO₂R^{6a}, wherein the aforementioned D groups other than —H are optionally substituted, or is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different;
wherein R⁴², R⁴³, Y, R³⁷, R^{6a}, Z³ and Z⁴ are as defined in Formula (I); and
with the proviso that Formula (V) excludes those compounds wherein
X is O (except when Z¹² is OC(S) or C(O)) wherein Z¹² is as defined in Formula (I);
X¹ is O;
V is an unsubstituted 5 or 6 membered aryl ring system or an unsubstituted 5 or 6 membered heteroaryl ring system containing between one and three heteroatoms;
Z is selected from the group consisting of O, S, NH and N (optionally substituted C₁-C₄alkyl); and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of Formula (V-A) and racemic mixtures, diastereomers and enantiomers thereof: 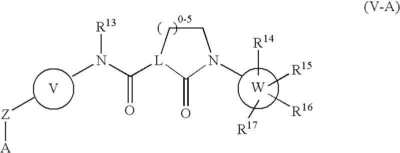
and N-oxides, hydrates, solvates, pharmaceutically acceptable salts, prodrugs and complexes thereof, wherein A, Z, V, W, R¹³, R¹⁴, R¹⁵, R¹⁶, R¹⁷ are as defined in Formula (I), and L is either —CH— or N. In a preferred embodiment of the compounds according to Formula (V-A), W is phenyl.

In a preferred embodiment of the present invention, the invention provides compounds of Formula (V-B) and racemic mixtures, diastereomers and enantiomers thereof: 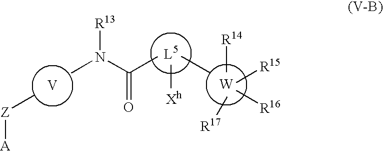
and N-oxides, hydrates, solvates, pharmaceutically acceptable salts, prodrugs and complexes thereof, wherein A, Z, V, W, R¹³, R¹⁴, R¹⁵, R¹⁶, R¹⁷ are as defined in Formula (I), and wherein L⁵ is selected from the group consisting of thiazolyl, phenyl and pyrazole, preferably pyrazole, and X^{h} is selected from the group consisting of absent, H, halogen, —NH₂, alkyl and —CF₃, preferably —CF₃.

According to another embodiment, the invention provides compounds of Formula (VI) and racemic mixtures, diastereomers and enantiomers thereof: 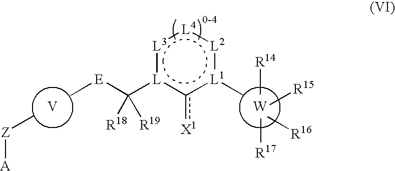
and N-oxides, hydrates, solvates, pharmaceutically acceptable salts, prodrugs and complexes thereof, wherein A, Z, V, E, W, R¹⁴, R¹⁵, R¹⁶, R¹⁶, R¹⁷, R¹⁸ and R¹⁹ are as defined in Formula (I); [symbol]
is a single or double bond;
X¹ is selected from the group consisting of O, S, CH₂, N—CN, N—O-alkyl, NH and N(C₁-C₆alkyl) when [symbol] is a double bond or
X¹ is selected from the group consisting of H, halogen, alkyl, alkenyl, alkynyl, CN, alkoxy, NH(alkyl) and alkyl-thio, when [symbol] is a single bond;
L and L¹ are independently selected from the group consisting of —CH—, —N—, —C(halogen)- and —C(C₁-C₆alkyl)-;
L² and L³ are independently selected from the group consisting of CH, CH₂, N, O and S;
L⁴ is selected from the group consisting of absent, CH, CH₂, N, O and S; and the group 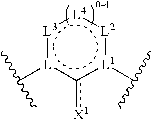
is aromatic or non-aromatic, provided that two 0 are not adjacent to each other; with the proviso that when [symbol] is a single bond, Formula (VI) excludes those compounds wherein
Z is O;
V is a 6 membered aryl ring system or a 6 membered heteroaryl ring system containing one heteroatom; and
X¹ is selected from the group consisting of H, halogen, alkyl, alkenyl, alkynyl, CN and alkoxy;
provided that this proviso does not exclude those compounds wherein W is substituted by either an alkenyl or alkynyl.
with the proviso that Formula (VI) excludes those compounds wherein
A is selected from the group consisting of 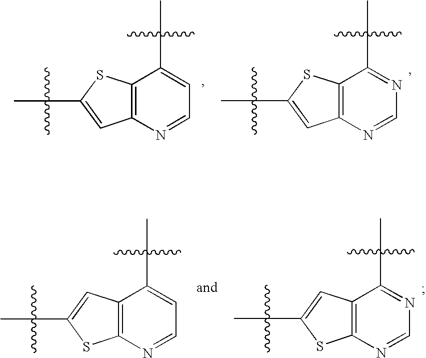
Z is —N(H)—, —N(C₁-C₆alkyl)-, —O—, —S—, —CH₂, —
R¹⁸ and R¹⁹ are H;
X¹ is O; and
W is unsubstituted; and
with the proviso that Formula (VI) excludes those compounds wherein
A is 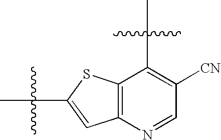
Z is NH or N(C₁-C₆alkyl);
R¹⁸ and R¹⁹ are H;
X¹ is O; and
W is unsubstituted; and
with the proviso that Formula (VI) excludes those compounds wherein
X¹ is O;
V is a 5 or 6 membered aryl ring system or a 5 or 6 membered heteroaryl ring system containing between one and three heteroatoms;
Z is selected from the group consisting of O, S, NH and N (optionally substituted C₁-C₄alkyl); and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of Formula (VI-A) and racemic mixtures, diastereomers and enantiomers thereof: 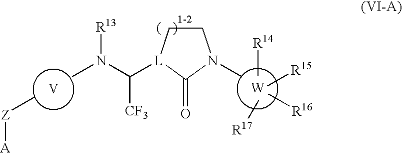
and N-oxides, hydrates, solvates, pharmaceutically acceptable salts, prodrugs and complexes thereof, wherein A, Z, V, W, R¹³, R¹⁴, R¹⁵, R¹⁶, R¹⁷ are as defined in Formula (I), and L is either —CH— or N.

In a preferred embodiment of the compounds according to Formula (VI-A), W is phenyl.

According to another embodiment, the invention provides compounds of Formula (VII) and racemic mixtures, diastereomers and enantiomers thereof: 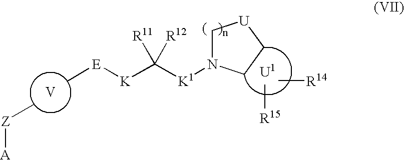
and N-oxides, hydrates, solvates, pharmaceutically acceptable salts, prodrugs and complexes thereof, wherein A, Z, V, E, R¹¹, R¹², R¹⁴ and R¹⁵ are as defined in Formula (I);
K and K¹ are independently selected from the group consisting of —C(O)—, —C(S)—, —C(NH)—, —C(NCN)— and —C(R¹⁸R¹⁹)—;
wherein R¹⁸ and R¹⁹ are as defined in Formula (I);
U is selected from the group consisting of O, S, SO₂, NH, and N(C₁-C₆alkyl), wherein the C₁-C₆alkyl is optionally substituted with a substituent selected from the group consisting of —OH, -alkoxy, amino, NH(C₁-C₆alkyl), N(C₁-C₆alkyl)₂, 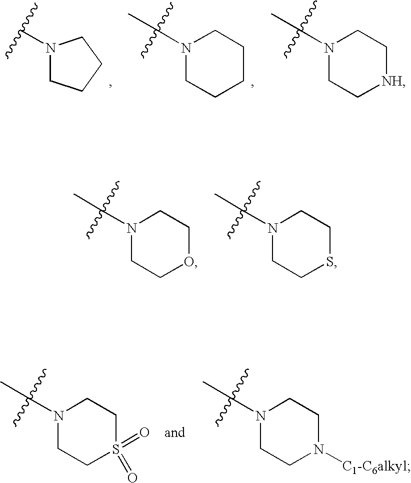
   and
U¹ is a ring system selected from the group consisting of cycloalkyl, substituted cycloalkyl, heterocyclyl, substituted heterocyclyl, aryl, substituted aryl, heteroaryl and substituted heteroaryl;
with the proviso that Formula (VII) excludes those compounds wherein
A is selected from the group consisting of 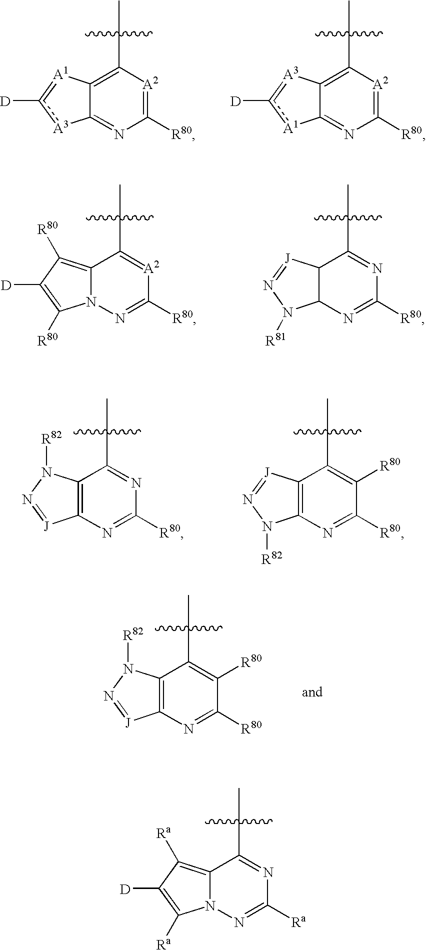
   wherein A¹, J, R⁸⁰, R⁸¹ and R⁸² are as defined in Formula (I);
A² is C(H) or C(CN);
A³ is selected from the group consisting of C(R^{a}) and N;
D and R^{a} are independently selected from the group consisting of H, halogen, NO₂, cyano, OR^{c}, NR^{c}R^{c}, CO₂R^{c}, C(O)NR^{c}R^{c}, SO₂R^{c}, SO₂NR^{c}R^{c}, NR^{c}SO₂R^{c}, NR^{c}C(O)R^{c}, NR^{c}CO₂R^{c}, —CO(CH₂)₀₋₄R^{c}, —CONH(CH₂)₀₋₄R^{c}, alkylaminoalkyl, alkylaminoalkynyl, C₁-C₆alkyl, substituted C₁-C₆alkyl, C₃-C₇cycloalkyl, substituted C₃-C₇cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl and substituted heterocycloalkyl; wherein
each R^{c} is independently selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl and substituted heterocycloalkyl;
K is selected from the group consisting of —C(O)—, —C(S)—, —C(NH)— and —C(NCN)—; and
K¹ is —C(O)—; and
with the proviso that Formula (VII) excludes those compounds wherein
A is selected from the group consisting of 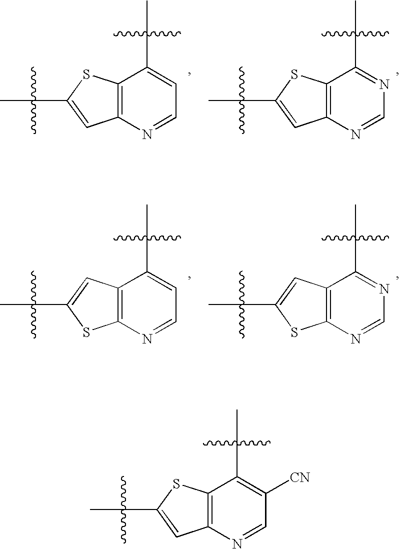
E is selected from the group consisting of —N(H)—, —N(C₁-C₆alkyl)- and —N(H)CH₂—;
Z is selected from the group consisting of —O—, —N(C(O)(C₁-C₆alkyl)), —S—, —CH₂—, —N(H)—, and —N(C₁-C₆alkyl); and
D is selected from the group consisting of —H, C₁-C₆ alkyl, C₃-C₁₀ cycloalkyl, —C(O)NR⁴²R⁴³, —Y—NR⁴²R⁴³, —NR⁴²C(═O)R⁴, —SO₂R⁴², —SO₂NR⁴²R⁴³, —NR³⁷SO₂R⁴², —NR³⁷S₂NR⁴²R⁴³, —C(═N—OR⁴²)R⁴³, —C(═NR⁴²)R⁴³, —NR³⁷C(═NR⁴²)R⁴³, —C(═NR⁴²)N³⁷R⁴³, —NR³⁷C(═NR⁴²)NR³⁷R⁴³, —C(O)R⁴², —CO₂R⁴², —C(O)(C₆-C₁₀ aryl), —Y—(C₆-C₁₀ aryl), —Y-(5-10 membered heterocyclyl), —CO₂R^{6a}, wherein the aforementioned D groups other than —H are optionally substituted;
wherein R⁴², R⁴³, Y, R³⁷ and R^{6a} are as defined in Formula (I); and
wherein R¹⁴ and R¹⁵ are both H unless K is —C(O)— and K¹ is —C(R¹⁸R¹⁹)—, or K and K¹ are both —C(R¹⁸R¹⁹)—; and
with the proviso that Formula (VII) excludes those compounds wherein
Z is selected from the group consisting of O, S, CH₂, N(Bn), N(H) and N (optionally substituted alkyl);
E is N(H) or N(alkyl);
K is selected from the group consisting of —C(O)—, —C(S)—, —C(NH)— and —C(NCN)—;
K¹ is —C(O)—; and
D is selected from the group consisting of H, halogen, NO₂, cyano, OR^{b}, NR^{b}R^{b}, CO₂R^{b}, C(O)NR^{b}R^{b}, SO₂R^{b}, SO₂NR^{b}R^{b}, NR^{b}SO₂R^{b}, NR^{b}C(O)R^{b}, NR^{b}CO₂R^{b}, —CO(CH₂)₁R^{b}, —CONH(CH₂)₁R^{b}, alkylaminoalkyl, alklaminoalkynyl, C₁-C₆alkyl, substituted C₁-C₆alkyl, C₃-C₇cycloalkyl, substituted C₃-C₇ cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl and substituted heterocycloalkyl;
wherein R^{b} is selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl and substituted heterocycloalkyl; and
with the proviso that Formula (VII) excludes those compounds wherein
V is a 5 or 6 membered aryl ring system or a 5 or 6 membered heteroaryl ring system containing between one and three heteroatoms;
Z is selected from the group consisting of O, S, NH and N (optionally substituted C₁-C₄alkyl);
K and K¹ are independently selected from the group consisting of —C(O)—, —C(NH)—, —C(NCN)— and —C(R¹⁸R¹⁹)—; and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of Formula (VII-A) and racemic mixtures, diastereomers and enantiomers thereof: 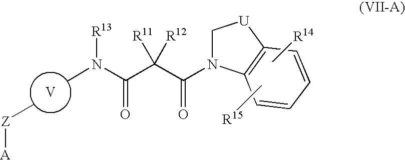
and N-oxides, hydrates, solvates, pharmaceutically acceptable salts, prodrugs and complexes thereof, wherein A, Z, V, U, R¹¹, R¹², R¹³, R¹⁴ and R¹⁵ are as defined in Formula (I).

In a preferred embodiment of the present invention, the invention provides compounds of Formula (VII-B) and racemic mixtures, diastereomers and enantiomers thereof: 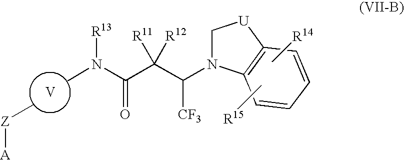
and N-oxides, hydrates, solvates, pharmaceutically acceptable salts, prodrugs and complexes thereof, wherein A, Z, V, U, R¹¹, R¹², R¹³, R¹⁴ and R¹⁵ are as defined in Formula (I).

According to another embodiment, the invention provides compounds of Formula (VIII) and racemic mixtures, diastereomers and enantiomers thereof: 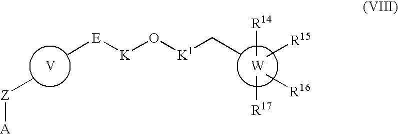
and N-oxides, hydrates, solvates, pharmaceutically acceptable salts, prodrugs and complexes thereof, wherein A, Z, V, E, R¹⁴, R¹⁵, R¹⁶ and R¹⁷ are as defined in Formula (I); and
K and K¹ are independently selected from the group consisting of —C(O)—, —C(S)—, —C(NH)—, —C(NCN)— and —C(R¹⁸R¹⁹)—;
wherein R¹⁸ and R¹⁹ are as defined in Formula (I);
with the proviso that Formula (VI II) excludes those compounds wherein
Z is O;
V is a 6 membered aryl ring system or a 6 membered heteroaryl ring system containing one heteroatom;
K is —C(O)— or —C(R¹⁸R¹⁹)—; and
K¹ is selected from the group consisting of —C(O)—, —C(S)— and —C(R¹⁸R¹⁹)—;
provided that this proviso does not exclude those compounds wherein W is substituted by either an alkenyl or alkynyl.
with the proviso that Formula (VI II) excludes those compounds wherein
Z is selected from the group consisting of S, O, NH and N (optionally substituted alkyl);
V is a 5 or 6 membered aryl ring system or a 5 or 6 membered heteroaryl ring system containing between one and three heteroatoms;
K is —C(O)— or —C(R¹⁸R¹⁹)—;
K¹ is —C(R¹⁸R¹⁹)—; and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl); and
R¹⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of Formula (VIII-A) and racemic mixtures, diastereomers and enantiomers thereof: 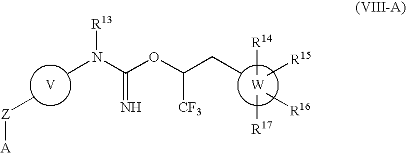
and N-oxides, hydrates, solvates, pharmaceutically acceptable salts, prodrugs and complexes thereof, wherein A, Z, V, W, R¹³, R¹⁴, R¹⁵, R¹⁶ and R¹⁷ are as defined in Formula (I).

In a preferred embodiment of the compounds according to Formula (VIII-A), W is phenyl.

According to another embodiment, the invention provides compounds of Formula (IX) and racemic mixtures, diastereomers and enantiomers thereof: 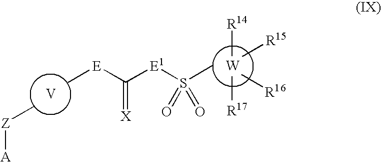
and N-oxides, hydrates, solvates, pharmaceutically acceptable salts, prodrugs and complexes thereof, wherein A, Z, V, E, X, W R¹⁴, R¹⁵, R¹⁶ and R¹⁷ are as defined in Formula (I); and E¹ is selected from the group consisting of —N(H)—, —N(C₁-C₆alkyl)-, —CH₂N(H)— and —N(H)CH₂—; with the proviso that Formula (IX) excludes those compounds wherein
Z is selected from the group consisting of S, O, NH and N (optionally substituted alkyl);
V is a 5 or 6 membered aryl ring system or a 5 or 6 membered heteroaryl ring system containing between one and three heteroatoms;
X is selected from the group consisting of O, S, NH, NOH, NOMe and NOEt;
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of Formula (IX-A) and racemic mixtures, diastereomers and enantiomers thereof: 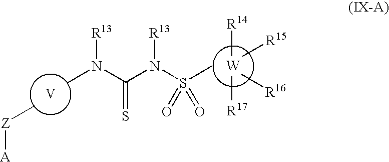
and N-oxides, hydrates, solvates, pharmaceutically acceptable salts, prodrugs and complexes thereof, wherein A, Z, V, R¹³, R¹⁴, R¹⁵, R¹⁶ and R¹⁷ are as defined in Formula (I).

In a preferred embodiment of the compounds according to Formula (IX-A), W is phenyl.

According to another embodiment, the invention provides compounds of Formula (X) and racemic mixtures, diastereomers and enantiomers thereof: 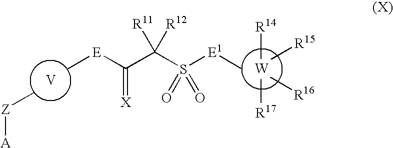
and N-oxides, hydrates, solvates, pharmaceutically acceptable salts, prodrugs and complexes thereof, wherein A, Z, V, E, X, W R¹⁴, R¹⁵, R¹⁶ and R¹⁷ are as defined in Formula (I); and E¹ is selected from the group consisting of —N(H)—, —N(C₁-C₆alkyl)-, —CH₂N(H)— and —N(H)CH₂—.

In a preferred embodiment of the present invention, the invention provides compounds of Formula (X-A) and racemic mixtures, diastereomers and enantiomers thereof: 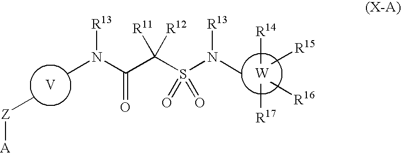
and N-oxides, hydrates, solvates, pharmaceutically acceptable salts, prodrugs and complexes thereof, wherein A, Z, V, R¹¹, R¹², R¹³, R¹⁴, R¹⁵, R¹⁶ and R¹⁷ are as defined in Formula (I).

In a preferred embodiment of the compounds according to Formula (X-A), W is phenyl.

According to another embodiment, the invention provides compounds of Formula (XI) and racemic mixtures, diastereomers and enantiomers thereof: 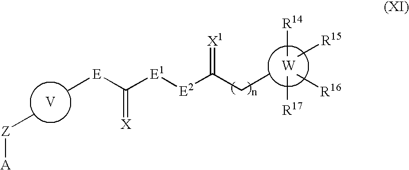
and N-oxides, hydrates, solvates, pharmaceutically acceptable salts, prodrugs and complexes thereof, wherein A, Z, V, E, X, W R¹⁴, R¹⁵, R¹⁶ and R¹⁷ are as defined in Formula (I);
n is 0, 1, 2, 3 or 4;
X¹ is selected from the group consisting of O, S, NH, NOH, NOMe, NOEt and NCN;
E¹ is selected from the group consisting of —N(H)—, —N(C₁-C₆alkyl)-, —CH₂N(H)— and —N(H)CH₂—; and
E² is selected from the group consisting of —N(H)—, —N(C₁-C₆alkyl)-, —CH₂N(H)— and —N(H)CH₂—; with the proviso that Formula (XI) excludes those compounds wherein
Z is selected from the group consisting of S, O, NH and N (optionally substituted alkyl);
V is a 5 or 6 membered aryl ring system or a 5 or 6 membered heteroaryl ring system containing between one and three heteroatoms;
X is selected from the group consisting of O, S, NH, NOH, NOMe and NOEt;
X¹ is O;
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

According to another embodiment, the invention provides compounds of Formula (XII) and racemic mixtures, diastereomers and enantiomers thereof: 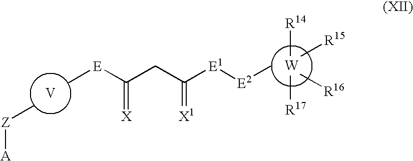
and N-oxides, hydrates, solvates, pharmaceutically acceptable salts, prodrugs and complexes thereof, wherein A, Z, V, E, X, W R¹⁴, R¹⁵, R¹⁶ and R¹⁷ are as defined in Formula (I);
E¹ is selected from the group consisting of —N(H)—, —N(C₁-C₆alkyl)-, —CH₂N(H)— and —N(H)CH₂—;
E² is selected from the group consisting of —N(H)—, —N(C₁-C₆alkyl)-, —CH₂N(H)— and —N(H)CH₂—; and
X¹ is selected from the group consisting of O, S, NH, NOH, NOMe, NOEt and NCN; with the proviso that Formula (XII) excludes those compounds wherein
Z is selected from the group consisting of S, O, NH and N (optionally substituted alkyl);
V is a 5 or 6 membered aryl ring system or a 5 or 6 membered heteroaryl ring system containing between one and three heteroatoms;
X and X¹ are O;
D is selected from the group consisting of —H, halogen, trihalomethyl, —CN, nitro, —OR^{e}, —N(R^{e})R^{e}, —S(O)₂R^{e}, —SO₂N(R^{e}) R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

According to another embodiment, the invention provides compounds of Formula (XIII) and racemic mixtures, diastereomers and enantiomers thereof: 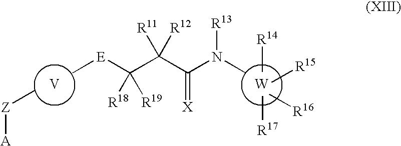
and N-oxides, hydrates, solvates, pharmaceutically acceptable salts, prodrugs and complexes thereof, wherein A, Z, V, E, X, W, R¹¹, R¹²R¹³, R¹⁴, R¹⁵, R¹⁶, R¹⁷, R¹⁸ and R¹⁹ are as defined in Formula (I);
with the proviso that Formula (XIII) excludes those compounds wherein
A¹ is —S—;
A2 is —CH— or N;
A³ is —CH—;
Z is selected from the group consisting of —N(H)—, —O—, —S— and —CH₂—;
X is O; and
W is C₃-C₁₀cycloalkyl; and
with the proviso that Formula (XIII) excludes those compounds wherein
Z is O;
V is a 6 membered aryl ring system or a 6 membered heteroaryl ring system containing one heteroatom;
E is —N(H)—;
R¹¹, R¹², R¹⁸ and R¹⁹ are each H; and
X is O;
provided that this proviso does not exclude those compounds wherein W is substituted by either an alkenyl or alkynyl; and
with the proviso that Formula (XIII) excludes those compounds wherein
Z is selected from the group consisting of S, O, NH and N (optionally substituted alkyl);
V is a 5 or 6 membered aryl ring system or a 5 or 6 membered heteroaryl ring system containing between one and three heteroatoms;
X is O;
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{e})R^{e}, and optionally substituted C₁-C₆alkyl;
provided that this proviso does not exclude those compounds wherein W is substituted by a halogen and either an alkenyl or alkynyl.

In a preferred embodiment of the present invention, the invention provides compounds of Formula (XIII-A) and racemic mixtures, diastereomers and enantiomers thereof: 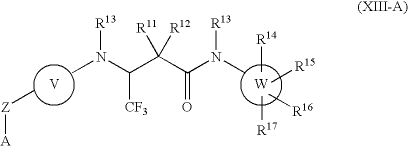
and N-oxides, hydrates, solvates, pharmaceutically acceptable salts, prodrugs and complexes thereof, wherein A, Z, V, W, R¹¹, R¹², R¹³, R¹⁴, R¹⁵, R¹⁶ and R¹⁷ are as defined in Formula (I).

In a preferred embodiment of the compounds according to Formula (XIII-A), W is phenyl.

According to another embodiment, the invention provides compounds of Formula (XIV) and racemic mixtures, diastereomers and enantiomers thereof: 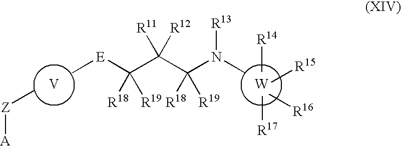
and N-oxides, hydrates, solvates, pharmaceutically acceptable salts, prodrugs and complexes thereof, wherein A, Z, V, E, X, W, R¹¹, R¹², R¹³, R¹⁴, R¹⁵, R¹⁶, R¹⁷, R¹⁸ and R¹⁹ are as defined in Formula (I); and
each R¹⁸ and R¹⁹ is independent of each other R¹⁸ and R¹⁹;
with the proviso that Formula (XIV) excludes those compounds wherein
A is selected from the group consisting of 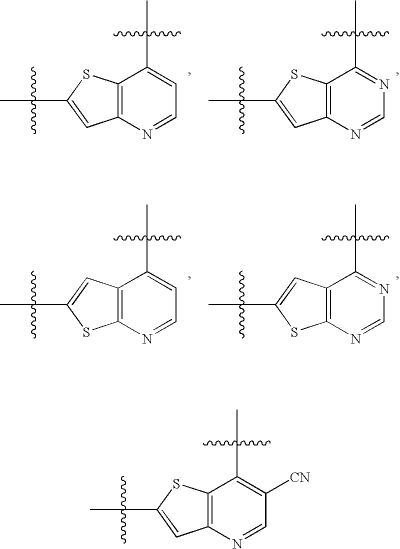
Z is O;
each R¹⁸ and R¹⁹ is H;
R¹⁷ is H; and
R¹⁴, R¹⁵ and R¹⁶ are independently selected from the group consisting of halogen, hydroxyl, cyano, nitro, trifluoromethyl, azido, —C(O)R^{g}, —C(O)OR^{g}, —CO(O)R^{g}, OC(O)OR^{g}, —NR^{h}C(O)Rⁱ, —C(O)NR^{h}Rⁱ, —NR^{h}Rⁱ, C₁-C₆alkyl, —(CH₂)₀₋₆(C₆-C₁₀aryl), —(CH₂)₀₋₆(5 to 10 membered heterocyclic), —(CH₂)₀₋₆O(CH₂)₂₋₆OR^{h} and —(CH₂)₀₋₆OR^{h}; wherein
each R^{g} is independently selected from the group consisting of H, C₁-C₁₀alkyl, C₃-C₁₀ cycloalkyl, —(CH₂)₀₋₆(C₆-C₁₀aryl) and —(CH₂)₀₋₆(5 to 10 membered heterocyclic); and
each R^{h} and Rⁱ is independently selected from H, —OR^{j}, C₁-C₆alkyl and C₃-C₁₀cycloalkyl; wherein

R^{j} is selected from the group consisting of H, OH, C₁-C₆alkyl, C₃-C₁₀cycloalkyl, —(CH₂)₀₋₆(C₆-C₁₀aryl), —(CH₂)₀₋₆(5 to 10 membered heterocyclic), —(CH₂)₀₋₆₀(CH₂)₂₋₆OR^{h}, —(CH₂)₀₋₆CN(CH₂)₀₋₆₀R^{h}, —(CH₂)₀₋₆CN(CH₂)₀₋₆R^{h} and —(CH₂)₀₋₆OR^{h}, wherein the alkyl, aryl and heterocyclic moieties of said R^{j} are unsubstituted or substituted with one or more substituents independently selected from halogen, hydroxyl, cyano, nitro, trifluoromethyl, azido, —C(O)R^{g}, —C(O)OR^{g}, —CO(O)R^{g}, OC(O)OR^{g}, —NR^{h}C(O)Rⁱ, —C(O)NR^{h}Rⁱ, —NR^{h}Rⁱ, C₁-C₆alkyl, —(CH₂)₀₋₆(C₆-C₁₀aryl), —(CH₂)₀₋₆(5 to 10 membered heterocyclic), —(CH₂)₀₋₆O(CH₂)₂₋₆₀R^{h} and —(CH₂)₀₋₆OR^{h}, and
with the proviso that Formula (XIV) excludes those compounds wherein
Z is O;
E is —N(H)— or —N(H)CH₂—; and
R¹⁴, R¹⁵, R¹⁶ and R¹⁷ are independently selected from the group consisting of —H, halogen, trihalomethyl, —O-trihalomethyl, —CN, —OR³, —OCF₃, —C(O)OR³, —C(O)NR³R³, —C(O)R³, —C₁-C₄ alkoxy, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, and a saturated or unsaturated three- to seven-membered carboxyclic or heterocyclic group, wherein T² is selected from the group consisting of —OH, —OMe and —OEt and wherein the aryl, heteroaryl, C₁-C₆ alkyl, C₂-C₆ alkenyl, and C₂-C₆ alkynyl are optionally substituted;
provided that this proviso does not exclude those compounds wherein W is substituted by either an alkenyl or alkynyl; and
with the proviso that Formula (XIV) excludes those compounds wherein
Z is selected from the group consisting of S, O, NH and N (optionally substituted alkyl);
V is a 5 or 6 membered aryl ring system or a 5 or 6 membered heteroaryl ring system containing between one and three heteroatoms;
R⁸⁰ is selected from the group consisting of H, halogen, —OR^{e}, —S(O)₀₋₂R^{e}, NO₂, —N(R^{e})R^{e}, and optionally substituted C₁-C₆alkyl; and
D is selected from the group consisting of —H, halogen, trihalomethyl, —CN, nitro, —OR^{e}, —N(R^{e})R^{e}, —S(O)₀₋₂R^{e}, —SO₂N(R^{e})R^{e}, —CO₂R^{e}, —C(O)N(R^{e})R^{e}, —N(R^{e})SO₂R^{e}, —N(R^{e})C(O)R^{e}, NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e}, wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P; and
M is selected from the group consisting of —O—, —S(O)₀₋₂—, NH and N (optionally substituted C₁-C₆alkyl);
provided that this proviso does not exclude those compounds wherein W is substituted by a halogen and either an alkenyl or alkynyl.

According to another embodiment, the invention provides compounds of Formula (XV) and racemic mixtures, diastereomers and enantiomers thereof: 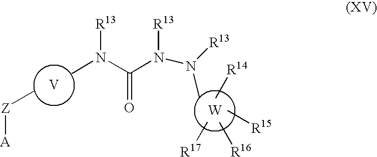
and N-oxides, hydrates, solvates, pharmaceutically acceptable salts, prodrugs and complexes thereof, wherein A, Z, V, W, R¹³, R¹⁴, R¹⁵, R¹⁶ and R¹⁷ are as defined in Formula (I).

In a preferred embodiment of the compounds according to Formula (XV), A is thienopyridine, Z is O and V is optionally substituted phenyl

In a preferred embodiment of the compounds according to Formula (XV), W is H or a cycloalkyl.

According to another embodiment, the invention provides compounds of Formula (XVI) and racemic mixtures, diastereomers and enantiomers thereof:

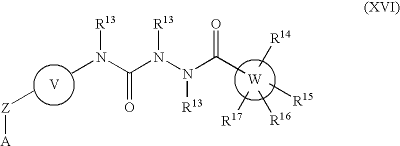

and N-oxides, hydrates, solvates, pharmaceutically acceptable salts, prodrugs and complexes thereof, wherein A, Z, V, W, R¹³, R¹⁴, R¹⁵, R¹⁶ and R¹⁷ are as defined in Formula (I).

In a preferred embodiment of the compounds according to Formula (XVI), A is thienopyridine, Z is O and V is optionally substituted phenyl

In a preferred embodiment of the compounds according to Formula (XVI), W is H or a cycloalkyl.

According to a preferred embodiment of the present invention, A is selected from the group consisting of 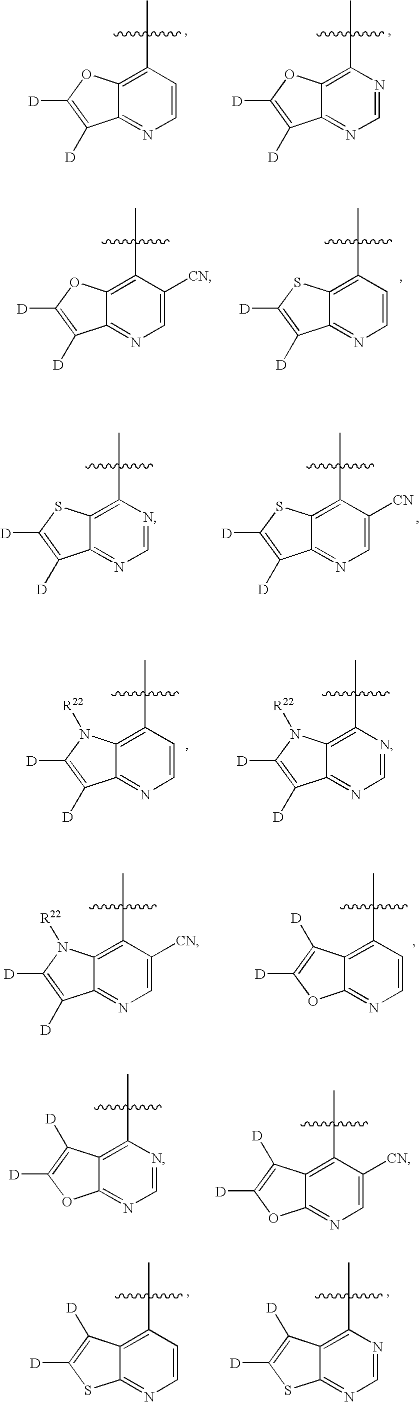 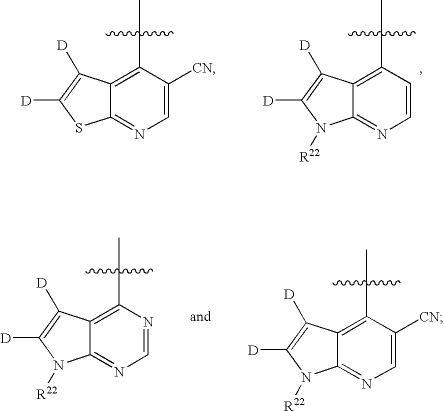
wherein R²² is selected from the group consisting of —H, —C₁-C₆alkyl, —Y-aryl, alkoxy, —CH₂—O-Me and -Bn.

According to another preferred embodiment of the present invention, A is selected from the group consisting of

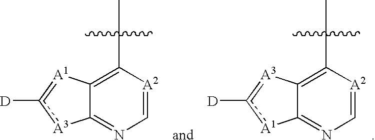

According to another preferred embodiment of the present invention, A is selected from the group consisting of

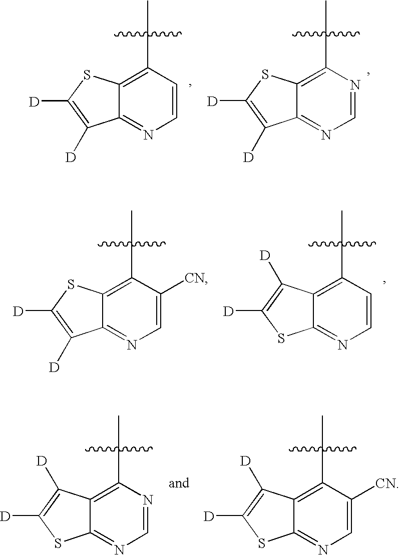

According to another preferred embodiment of the present invention, A is selected from the group consisting of

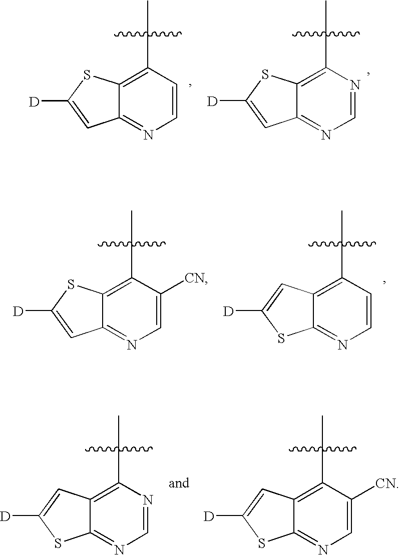

According to another preferred embodiment of the present invention A is

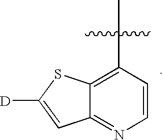

According to another preferred embodiment of the present invention, D is defined by the group R⁷, wherein R⁷ is selected from the group consisting of —H, halogen, C₁-C₆ alkyl, C₃-C₁₀ cycloalkyl, —C(O)NR⁴²R⁴³, —C(O)(C₆-C₁₀ aryl), —C(O)(heterocyclyl), —C(O)(heteroaryl), —Y—(C₆-C₁₀ aryl), —Y-(5-10 membered heterocyclyl), —Y-(heteroaryl), —S-aryl, —S—C₁-C₆ alkyl, —SO—C₁-C₆ alkyl, —SO₂—C₁-C₆ alkyl, —Y—NR⁴²R⁴³, —SO₂NR⁴²R⁴³ and —C(O)OR^{6a}, wherein the aforementioned R⁷ groups other than —H and halogen are optionally substituted.

According to another preferred embodiment of the present invention, D is defined by the group R⁷, wherein R⁷ is selected from the group consisting of —H, —C(O)NR⁴²R⁴³, —Y-(5 to 10 membered heterocyclyl), —Y—(C₆-C₁₀ aryl), —Y-(heteroaryl), —Y—NR⁴²R⁴³, SO₂NR⁴²R⁴³, and C(O)OR⁴², wherein the aforementioned R⁷ groups other than —H are optionally substituted.

According to another preferred embodiment of the present invention, R⁷ is selected from the group consisting of —(CH₂)ₙ(5 to 10 membered heterocyclyl), —C(O)NR⁴²R⁴³, —SO₂NR⁴²R⁴³ and —CO₂R⁴², wherein said R⁷ group —(CH₂)ₙ(5 to 10 membered heterocyclyl) is optionally substituted.

According to another preferred embodiment of the present invention, R⁷ is selected from the group consisting of —(CH₂)ₙ(5 to 10 membered heterocyclyl), and —C(O)NR⁴²R⁴³.

According to another preferred embodiment of the present invention, R⁷ is —C(O)NR⁴²R⁴³, wherein R⁴² and R⁴³ are independently selected from H, (C₁-C₆)alkyl, (C₃-C₁₀)cycloalkyl, —(CH₂)ₙ(C₃-C₁₀ cycloalkyl), —(CH₂)ₙ(C₆-C₁₀ aryl), —(CH₂)ₙ(5 to 10 membered heterocyclyl), —(CH₂)ₙO(CH₂)ᵢOR³⁷, —(CH₂)ₙOR³⁷, wherein n is an integer from 0 to 6, i is an integer from 2 to 6, and the alkyl, aryl and heterocyclyl moieties of said R⁴² and R⁴³ groups are unsubstituted or substituted with one or more substituents independently selected from R³⁸, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, where R⁴² and R⁴³ are not both bonded to the nitrogen directly through an oxygen.

According to another preferred embodiment of the present invention, R⁷ is —C(O)NR⁴²R⁴¹, wherein R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl, piperidinyl, piperazinyl, morpholinyl, thiomorpholinyl, isoquinolinyl, or dihydroisoquinolinyl ring, wherein said pyrrolidinyl, piperidinyl, piperazinyl, morpholinyl, thiomorpholinyl, isoquinolinyl, or dihydroisoquinolinyl ring are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl, piperidinyl, piperazinyl, morpholinyl, or thiomorpholinyl ring, wherein said pyrrolidinyl, piperidinyl, piperazinyl, morpholinyl, or thiomorpholinyl rings are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl or piperidinyl ring, wherein said pyrrolidinyl or piperidinyl ring are optionally substituted.

According to another preferred embodiment of the present invention, R⁷ is —C(O)NR⁴²R⁴³, wherein R⁴² and R⁴³ are taken together with the nitrogen to which they are attached to form a pyrrolidinyl ring, wherein said pyrrolidinyl ring is optionally substituted.

According to another preferred embodiment of the present invention, R⁷ is —C(O)NR⁴²R⁴³, wherein R⁴² and R⁴³ are taken together with the nitrogen to which they are attached to form a pyrrolidin-1-yl ring, wherein said pyrrolidin-1-yl is optionally substituted.

According to another preferred embodiment of the present invention, R⁷ is —(CH₂)ₙ(5 to 10 membered heterocyclyl) group, wherein said —(CH₂)ₙ(5 to 10 membered heterocyclyl) group is optionally substituted.

According to another preferred embodiment of the present invention, R⁷ is a —(CH₂)ₙ(5-8 membered heterocyclyl) group, wherein said —(CH₂)ₙ(5-8 membered heterocyclyl) group is optionally substituted.

According to another preferred embodiment of the present invention, R⁷ is a —(CH₂)ₙ(5 or 6 membered heterocyclyl) group, wherein said —(CH₂)ₙ(5 or 6 membered heterocyclyl) group is optionally substituted.

According to another preferred embodiment of the present invention, R⁷ is a —(CH₂)ₙ(5 membered heterocyclyl) group, wherein said —(CH₂)ₙ(5 membered heterocyclyl) group is optionally substituted.

According to another preferred embodiment of the present invention, R⁷ is —(CH₂)ₙthiazolyl, wherein n is an integer from 0 to 6, and said —(CH₂)ₙthiazolyl is optionally substituted.

According to another preferred embodiment of the present invention, R⁷ is a thiazolyl, wherein said thiazolyl is optionally substituted.

According to another preferred embodiment of the present invention, R⁷ is an imidazolyl, wherein said imidazolyl is optionally substituted.

According to another preferred embodiment of the present invention, R⁷ is selected from the group consisting of imidazolyl, oxazolyl, oxadiazolyl, isoxazolyl, thiazolyl and thiadiazolyl, wherein the imidazolyl, oxazolyl, oxadiazolyl, isoxazolyl, thiazolyl and thiadiazolyl, is optionally substituted.

According to another preferred embodiment of the present invention, R⁷ is selected from the group consisting of halo, —CO₂H, —CONH₂ and —CSNH₂.

According to another preferred embodiment of the present invention, R⁷ is a heteroaryl group optionally substituted by one or more moiety selected from the group consisting of halo, cyano, nitro, trifluoromethoxy, trofluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁷, —OR³⁷, —SO₂NR³⁶R³⁹, (C₁-C₆)alkyl, (C₃-C₁₀)cycloalkyl, —(CH₂)ⱼO(CH₂)ᵢNR³⁶R³⁹, —(CH₂)ₙO(CH₂)ᵢOR³⁹, —(CH₂)ₙOR³⁷, —S(O)ⱼ(C₁-C₆ alkyl), —(CH₂)ₙ(C₆-C₁₀ aryl), —(CH₂)ₙ(5 to 10 membered heterocyclyl), —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5 to 10 membered heterocyclyl), —C(O)(CH₂)ₙ(5 to 10 membered heterocyclyl), —(CH₂)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, (CH₂)ⱼNR³⁹(CH₂)ₙO(CH₂)ᵢOR³⁷, (CH₂)ⱼNR³⁹(CH₂)ᵢS(O)ⱼ(C₁-C₆ alkyl), —(CH₂)ⱼNR³⁹, —(CH₂)ₙR³⁶, —SO₂(CH₂)ₙ(C₆-C₁₀ aryl), and —SO₂(CH₂)ₙ(5 to 10 membered heterocyclyl), wherein j is an integer from 0 to 2, n is an integer from 0 to 6, i is an integer from 2 to 6, the —(CH₂)ᵢ— and —(CH₂)ₙ— moieties of the said substituent groups optionally include a carbon-carbon double or triple bond where n is an integer between 2 and 6, and the alkyl, aryl and heterocyclyl moieties of the substituent groups are unsubstituted or substituted with one or more substituents independently selected from halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)NR³⁶R³⁹, (C₁-C₆)alkyl, (C₃-C₁₀)cycloalkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer from 0 to 6 and i is an integer from 2 to 6, and wherein R³⁶ and R³⁹ are independently selected from the group consisting of H, —OH, (C₁-C₆)alkyl, (C₃-C₁₀)cycloalkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷, wherein n is an integer from 0 to 6 and i is an integer from 2 to 6, and the alkyl, aryl and heterocyclyl moieties of the R³⁶ and R³⁹ groups are unsubstituted or substituted with one or more substituents independently selected from hydroxy, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)N7R⁴¹, —N7R⁴¹, (C₁-C₆)alkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷, wherein n is an integer from 0 to 6 and i is an integer from 2 to 6, where when R³⁶ and R³⁹ are both attached to the same nitrogen, then R³⁶ and R³⁹ are not both bonded to the nitrogen directly through an oxygen.

According to another preferred embodiment of the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted. Preferably R⁷ is —(CH₂)ₙ(C₆-C₁₀ aryl) and —(CH₂)ₙ(5 to 10 membered heterocyclyl), optionally substituted, more preferably phenyl or pyridyl, optionally substituted.

According to another preferred embodiment of the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted.

According to another preferred embodiment of the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted by tert-butyl-dimethyl-silanyl and 1 to 3 R³⁸ groups.

According to another preferred embodiment of the present invention, R⁷ is selected from the group consisting of —C(O)NR⁴²R⁴³, —(CH₂)ₙNR⁴²R⁴³, —NR⁴²C(═O)R⁴³, —SO₂R⁴², —SO₂NR⁴²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, —C(═NR⁴²)NR³⁷R⁴³, —NR³⁷C(═NR⁴²)N³⁷R⁴³, —C(O)R⁴², —CO₂R⁴², wherein each R⁴² and R⁴³ is independently selected from the group consisting of H, (C₁-C₆)alkyl, —(CH₂)ₙ(C₃-C₁₀)cycloalkyl), —(CH₂)ₙ(C₆-C₁₀ aryl), —(CH₂)ₙ(5 to 10 membered heterocyclyl), —(CH₂)ₙO(CH₂)ᵢOR³⁷, —(CH₂)ₙOR³⁷, wherein n is an integer from 0 to 6 and i is an integer from 2 to 6, and the alkyl, aryl and heterocyclyl moieties of the foregoing R⁴² and R⁴ groups are optionally substituted by 1 to 3 substituents independently from R³⁸, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, with the proviso that R⁴² and R⁴³ are not both bonded to the nitrogen directly through an oxygen.

According to another preferred embodiment of the present invention, R⁷ is selected from the group consisting of —C(O)NR⁴²R⁴³, —SO₂R⁴², —SO₂NR⁴²R⁴³, —C(═N—OR⁴²)R⁴³ and —C(═NR⁴²)R⁴³.

According to another preferred embodiment of the present invention, R⁷ is —C(O)NR⁴²R⁴³, wherein each R⁴² and R⁴³ is independently selected from the group consisting of H, (C₁-C₆)alkyl, —(CH₂)ₙOR³⁷, wherein n is an integer from 0 to 6 and the alkyl moiety of the foregoing R⁴² and R⁴³ groups are optionally substituted by 1 to 3 substituents independently from halo, cyano, trifluoromethyl, —C(O)R⁴⁰, —NR³⁷C(O)R⁴¹, —C(O)NR³⁷R⁴¹, —NR³⁷R⁴¹, (C₁-C₆)alkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR₃₇, wherein n is an integer from 0 to 6 and i is an integer from 2 to 6, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, with the proviso that R⁴² and R⁴³ are not both bonded to the nitrogen directly through an oxygen.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl, pyrrolidinyl, piperidinyl, piperazinyl or morpholinyl ring, wherein said C₅-C₉ azabicyclic, aziridinyl, azetidinyl, pyrrolidinyl, piperidinyl, piperazinyl or morpholinyl ring are unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl or pyrrolidinyl ring, wherein said C₅-C₉ azabicyclic, aziridinyl, azetidinyl or pyrrolidinyl ring are unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic, azetidinyl or pyrrolidinyl ring, wherein said C₅-C₉ azabicyclic, azetidinyl or pyrrolidinyl ring are unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a C₅-C₉ azabicyclic ring, wherein said C₅-C₉ azabicyclic ring is unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a azetidinyl ring, wherein said azetidinyl ring is unsubstituted or substituted with 1 to 5 R³⁸ substituents.

According to another preferred embodiment of the present invention, R⁷ is —C(O)NR⁴²R⁴³, wherein R⁴² and R⁴³ are taken together with the nitrogen to which they are attached to form a pyrrolidinyl ring, wherein said pyrrolidinyl ring is unsubstituted or substituted with 1 to 5 R³⁸ substituents.

According to another preferred embodiment of the present invention, R⁷ is selected from the group consisting of —H, halogen, nitro, azido, —NR^{6a}R^{6b}, —NR^{6a}SO₂R^{6b}, —NR^{6a}C(O)R^{6b}, —OC(O)R^{6b}, —NR^{6a}C(O)OR^{6b}, —OC(O)NR^{6a}R^{6b}, —OR^{6a}, —SR^{6a}, S(O)R^{6a}, —SO₂R^{6a}, —SO₃R^{6a} SO₂NR^{6a}R^{6b}, —COR^{6a}, —CO₂R^{6a}, —CONR^{6a}R⁶b, —(C₁-C₄)fluoroalkyl, —(C₁-C₄)fluoroalkoxy, —(CZ³Z⁴)ₐCN, and a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C¹—C⁶)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶))ᵤ—(C₅-C₆)cycloalkenyl, —(CZ⁵Z⁶)ᵤ-aryl, —(CZ⁵Z⁶)ᵤ-heterocycle, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of halogen, cyano, nitro, tetrazolyl, guanidino, amidino, methylguanidino, azido, —C(O)Z⁷, —OC(O)NH₂, —OC(O) NHZ⁷, —OC(O)NZ⁷Z⁸, —NHC(O)Z⁷, —NHC(O)NH₂, —NHC(O)NHZ⁷, —NHC(O)NZ⁷Z⁸, C(O)OH, —C(O)OZ⁷, —C(O)NH₂, —C(O)NHZ⁷, —C(O)NZ⁷Z⁸, —P(O)₃H₂, —P(O)₃(Z⁷)₂, —S(O)₃H, —S(O)Z⁷, —S(O)₂Z⁷, —S(O)₃Z⁷, -Z⁷, —OZ⁷, —OH, —NH₂, —NHZ⁷, —NZ⁷Z⁸, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, —(CZ⁹Z¹⁰)ᵣNZ⁷Z⁸, —X⁶(CZ⁹Z¹⁰)ᵣ—(C₃-C₈)cycloalkyl, —X⁶(CZ⁹Z¹¹)ᵣ-(C₅-C₈)cycloalkenyl, —X⁶(CZ⁹Z¹⁰)-aryl and —X⁶(CZ⁹Z¹⁰)ᵣ-heterocycle, wherein
r is 1, 2, 3 or 4;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of an alkyl of 1 to 12 carbon atoms, an alkenyl of 2 to 12 carbon atoms, an alkynyl of 2 to 12 carbon atoms, a cycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, or
Z⁷ and Z⁸ together may optionally form a heterocycle;
Z⁹ and Z¹⁰ are independently selected from the group consisting of H, F, a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl].

According to another preferred embodiment of the present invention, R⁷ is selected from the group consisting of —H, —Y-(aryl), —Y-(heteroaryl) and C(O)-heterocyclyl, each of which, except for —H, is optionally substituted.

According to another preferred embodiment of the present invention, R⁷ is selected from the group consisting of —H, —Y-(aryl) and —Y-(heteroaryl), each of which, except for —H, is optionally substituted.

According to another preferred embodiment of the present invention, R⁷ is -aryl-(CH₂)₀₋₂N(R¹³)—(CH₂)₀₋₆—O—(CH₂)₀₋₆ or -heteroaryl-(CH₂)₀₋₂N(R¹³)—(CH₂)₀₋₆—O—(CH₂)₀₋₆, preferably -aryl-(CH₂)—N(H)—(CH₂)₂—O—(CH₂) or -heteroaryl-(CH₂)—N(H)—(CH₂)₂—O—(CH₂), and more preferably pyridine-(CH₂)—N(H)—(CH₂)₂—O—(CH₂).

According to another preferred embodiment of the present invention R⁷ is —(CH₂)₁₋₂N(H)—C₃-C₇cycloalkyl. Preferrably the C₃-C₇cycloalkyl is a C₃cycloalkyl.

According to another preferred embodiment of the present invention, R⁷ is selected from the group consisting of 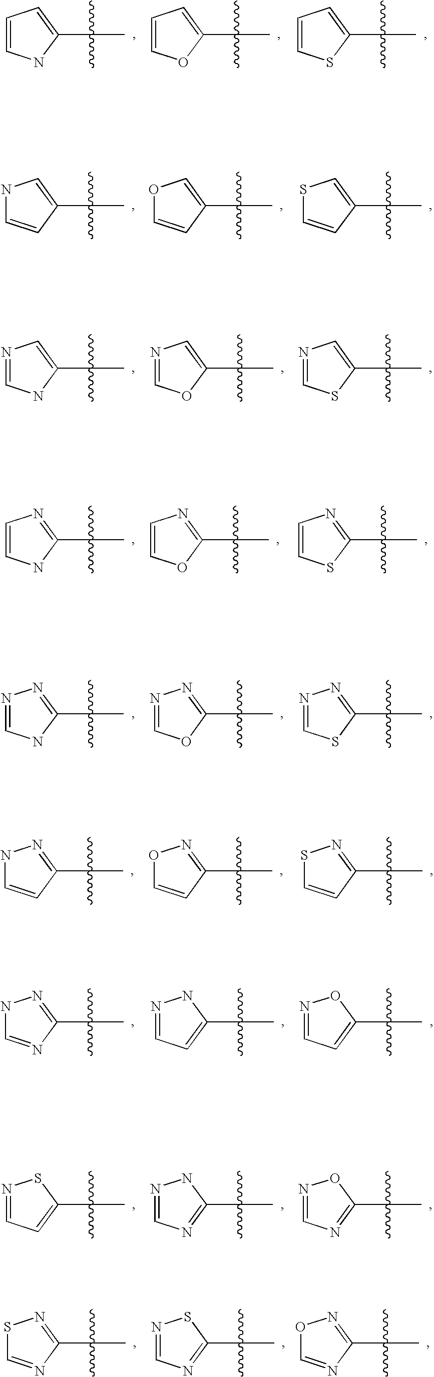 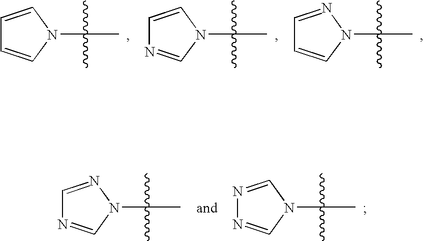
wherein the members of said group are optionally substituted.

According to another preferred embodiment of the present invention, R⁷ is selected from the group consisting of 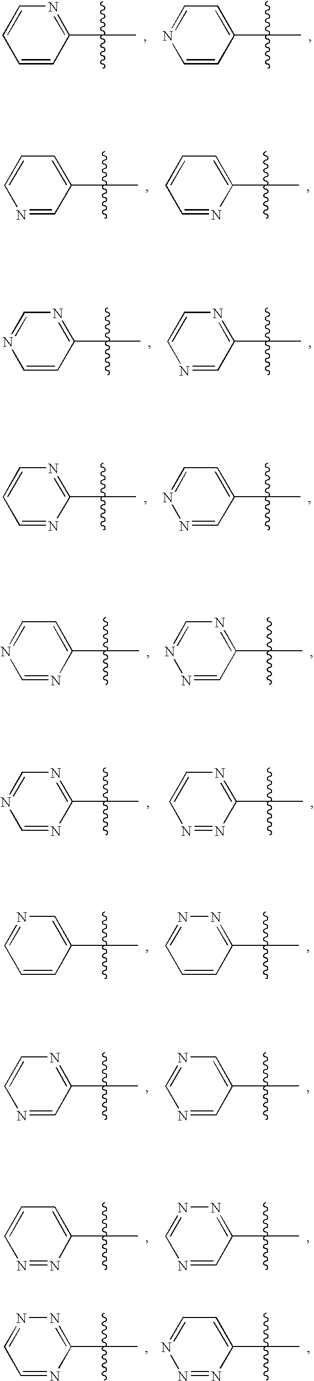 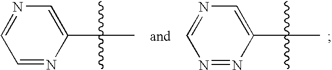
wherein the members of said group are optionally substituted.

According to another preferred embodiment of the present invention, R⁷ is selected from the group consisting of

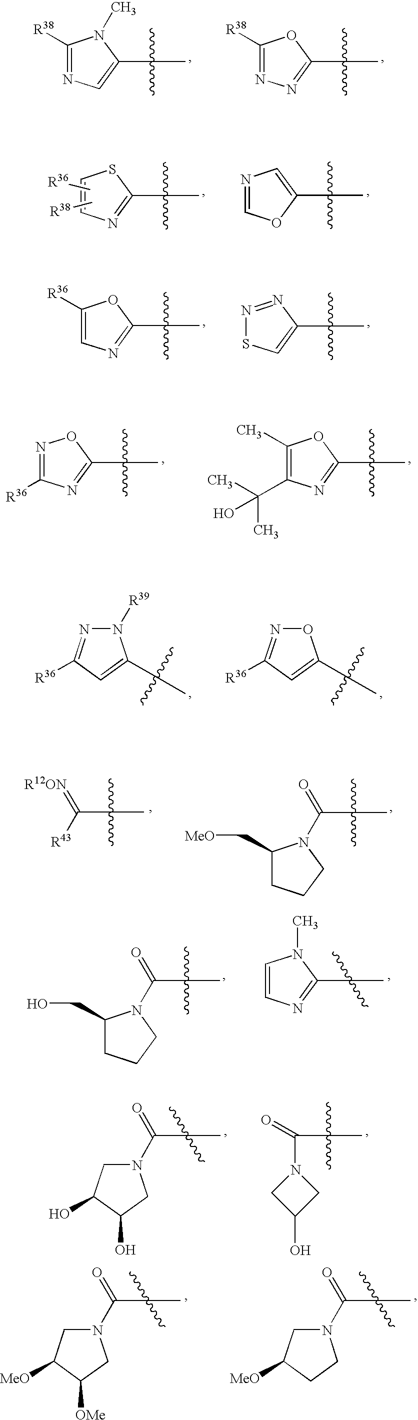

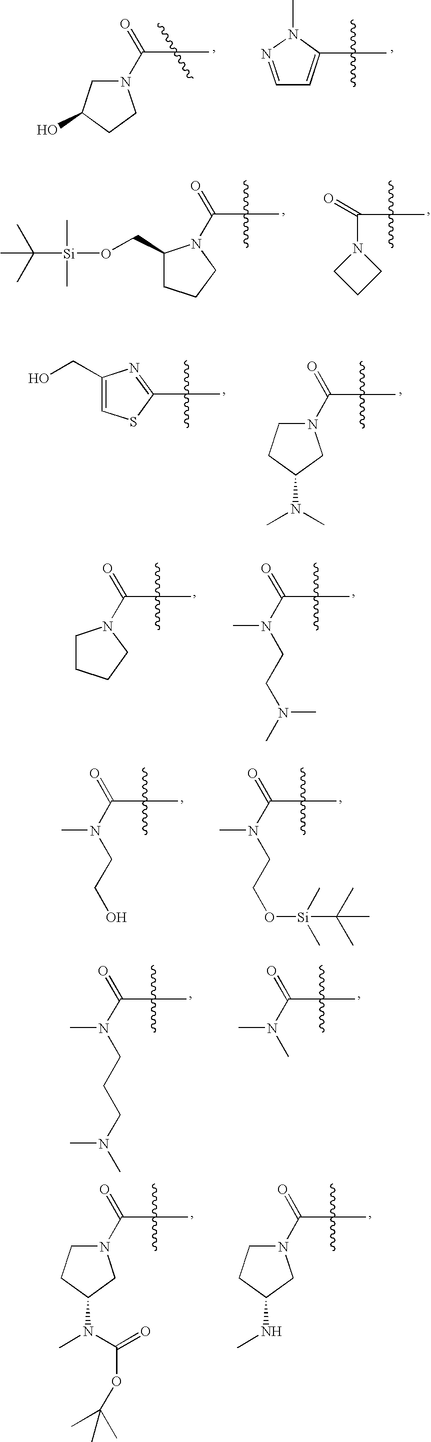

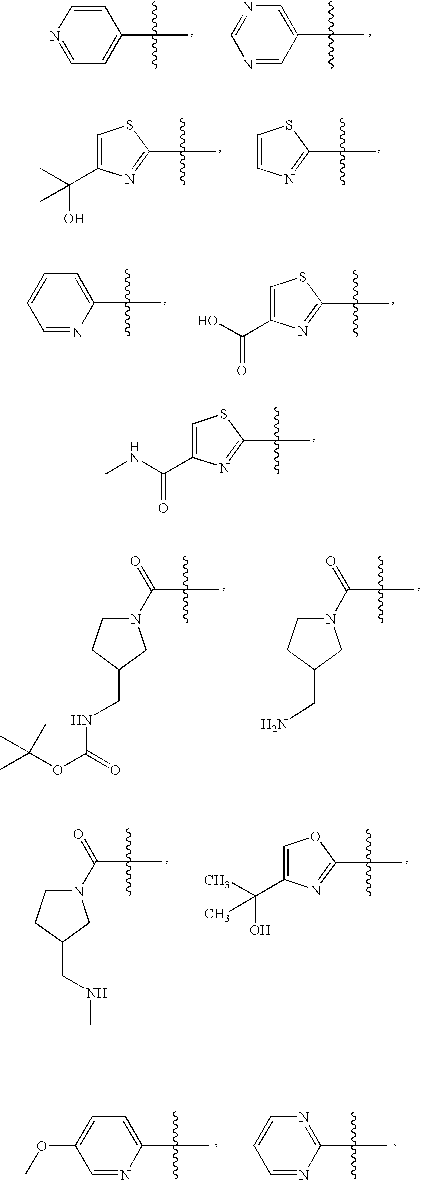

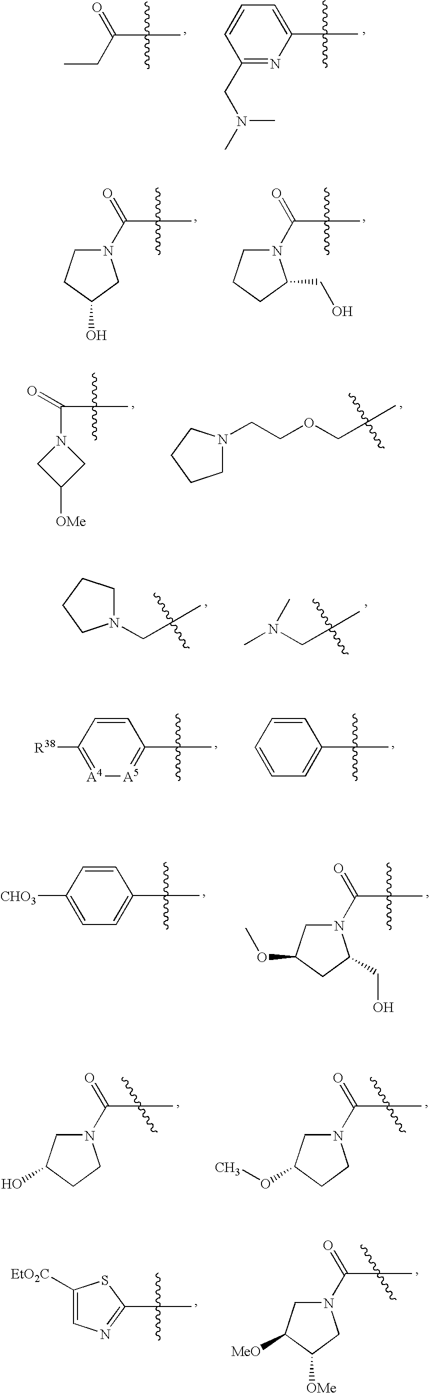

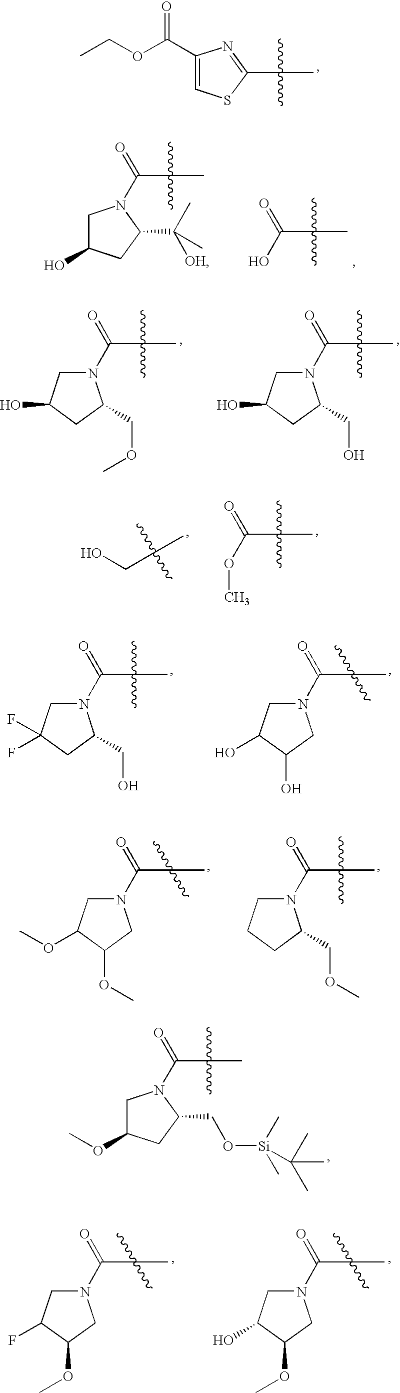

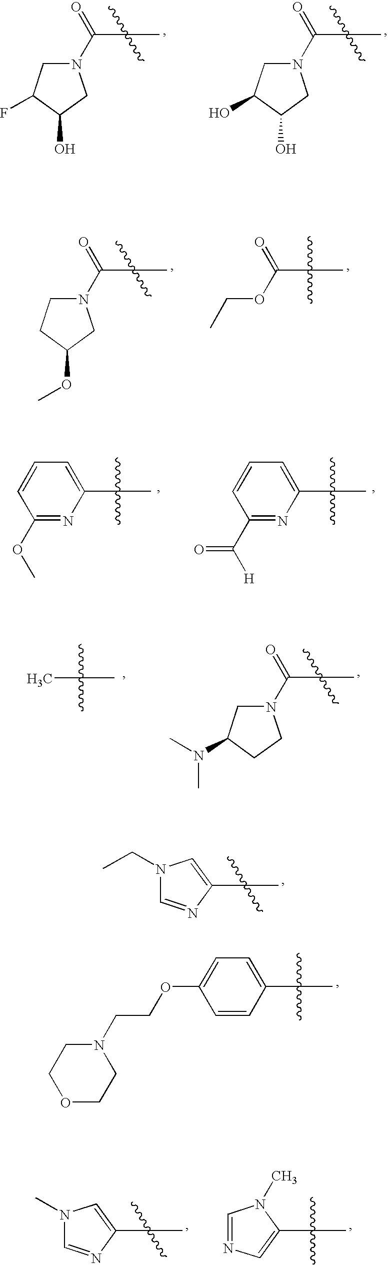

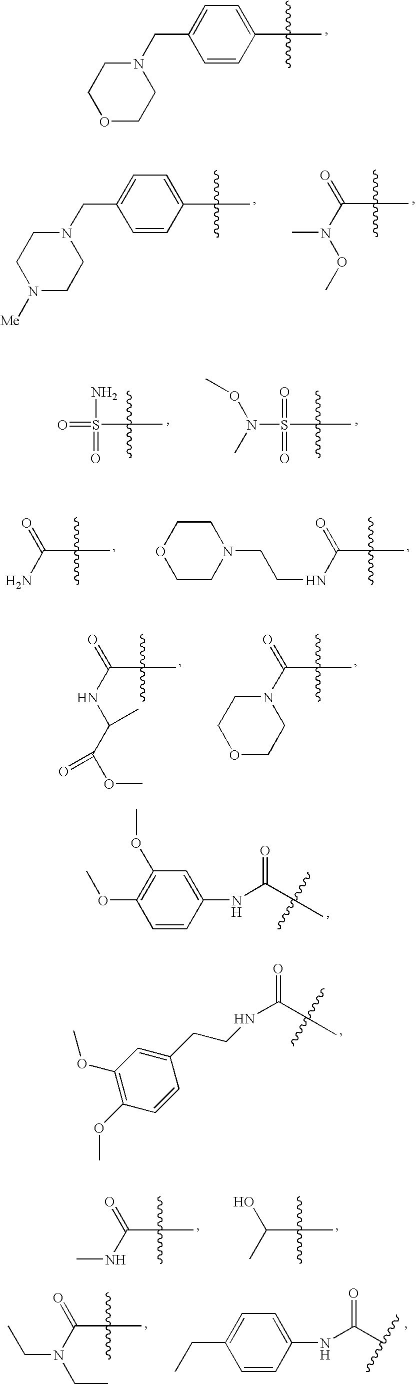

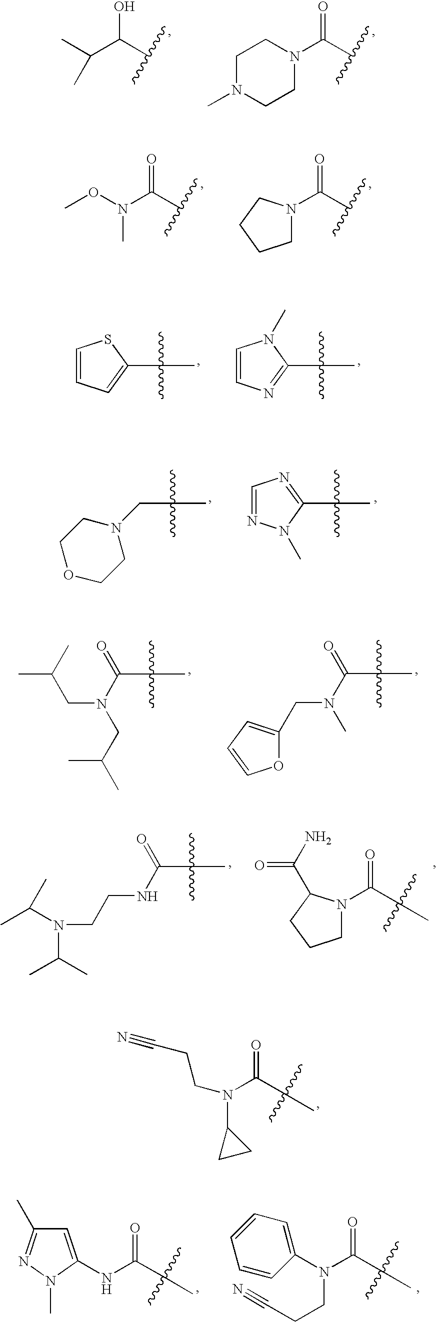

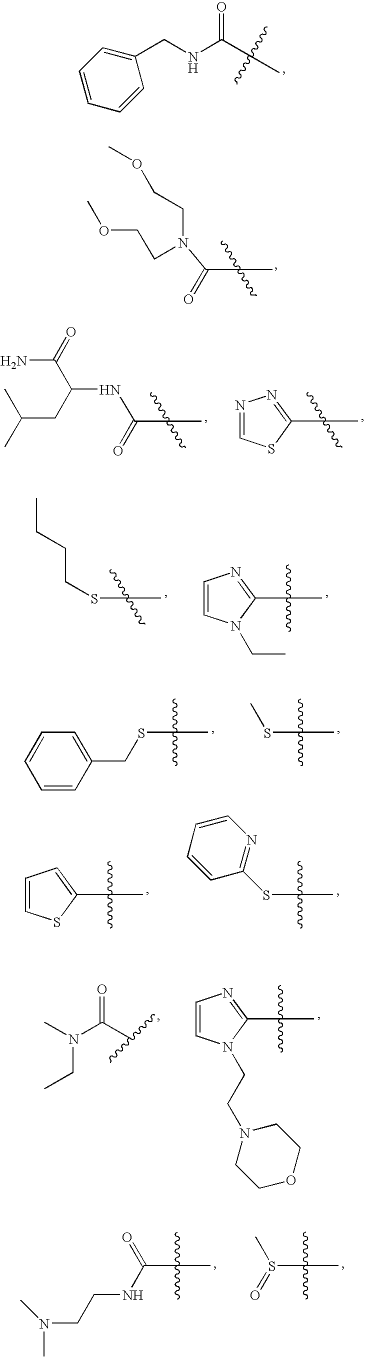

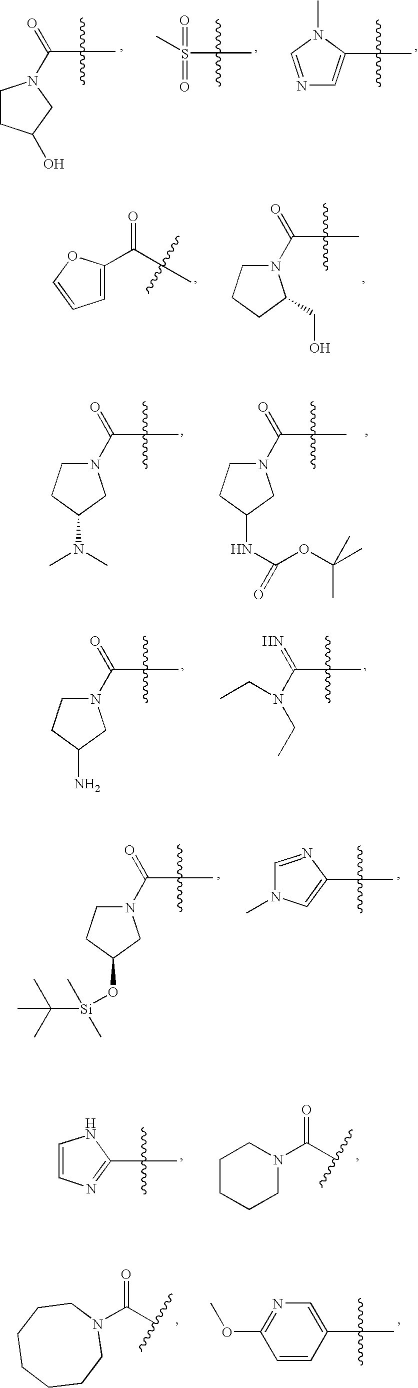

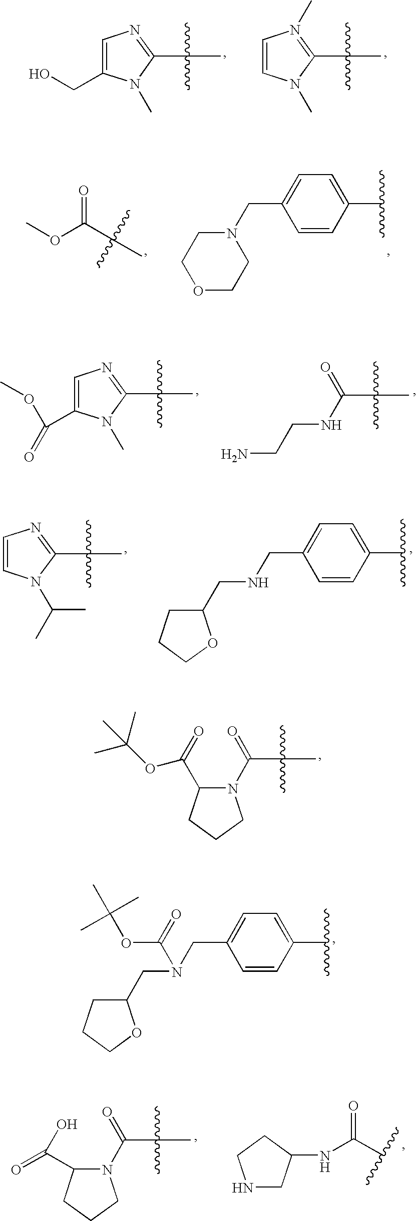

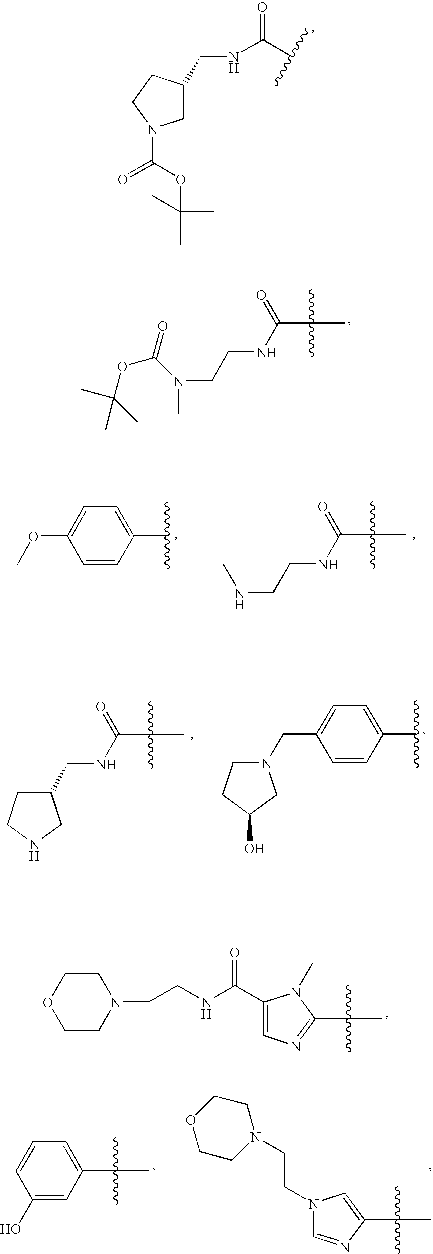

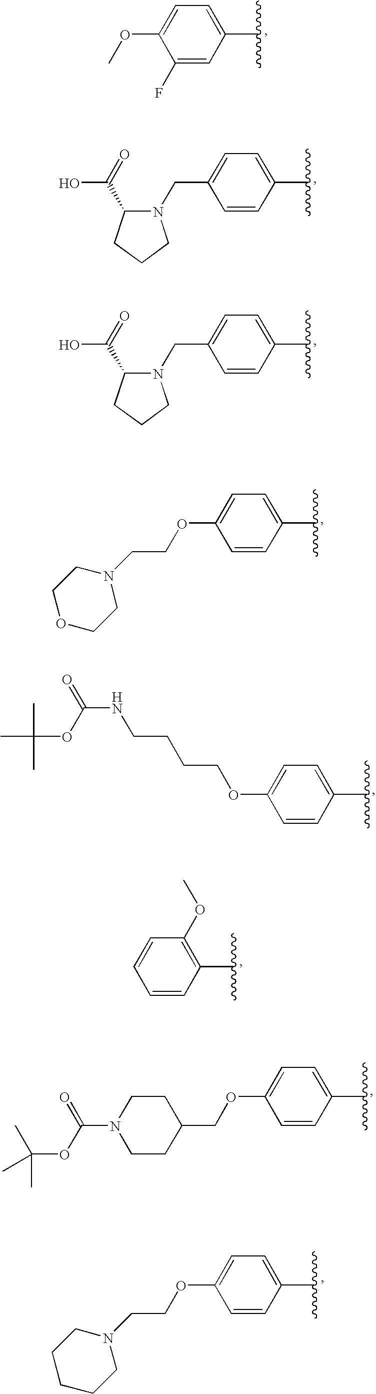

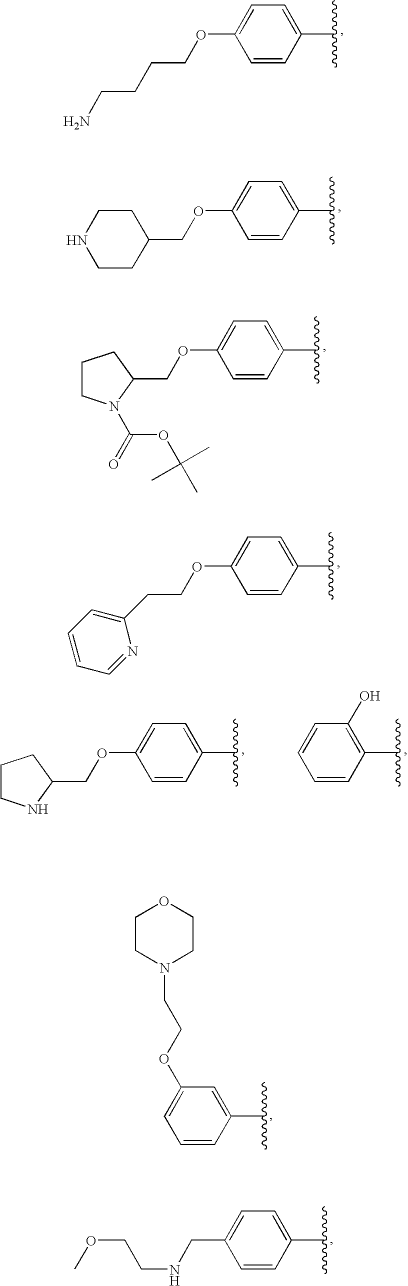

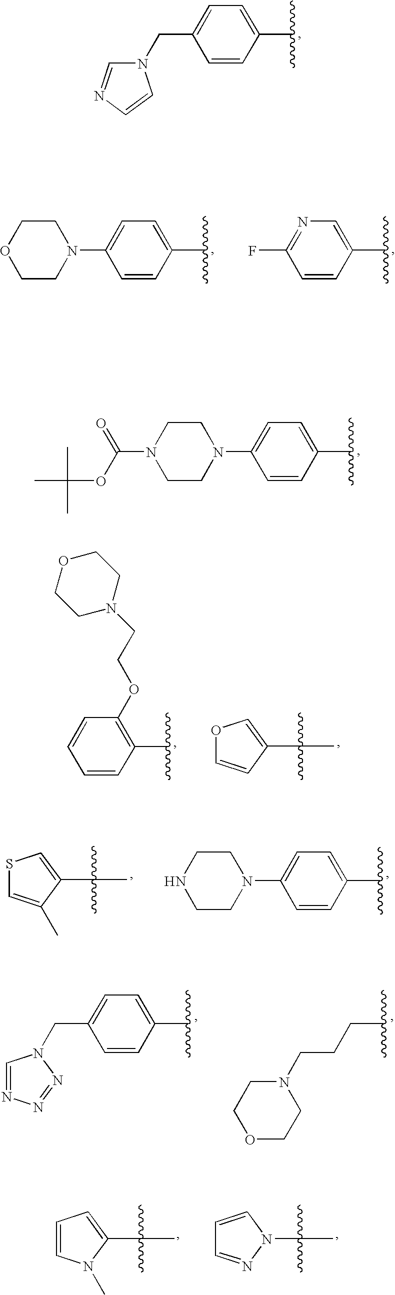

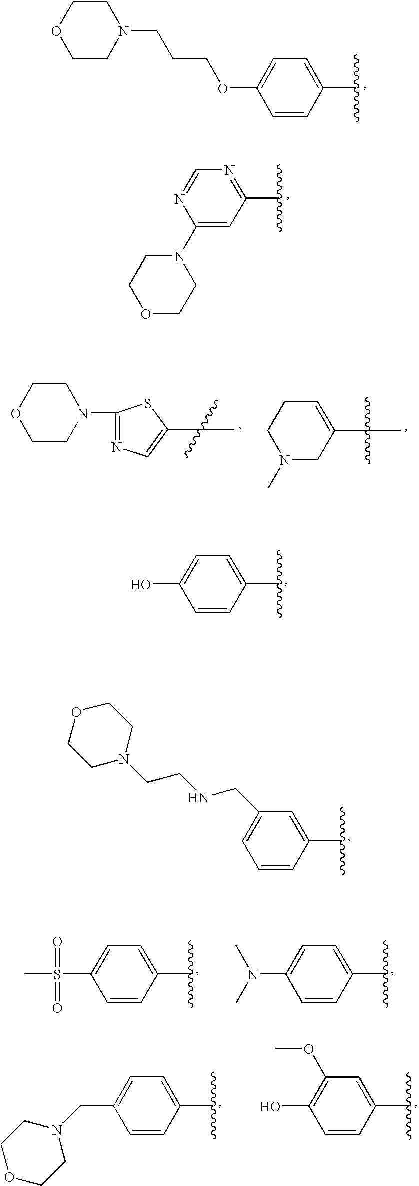

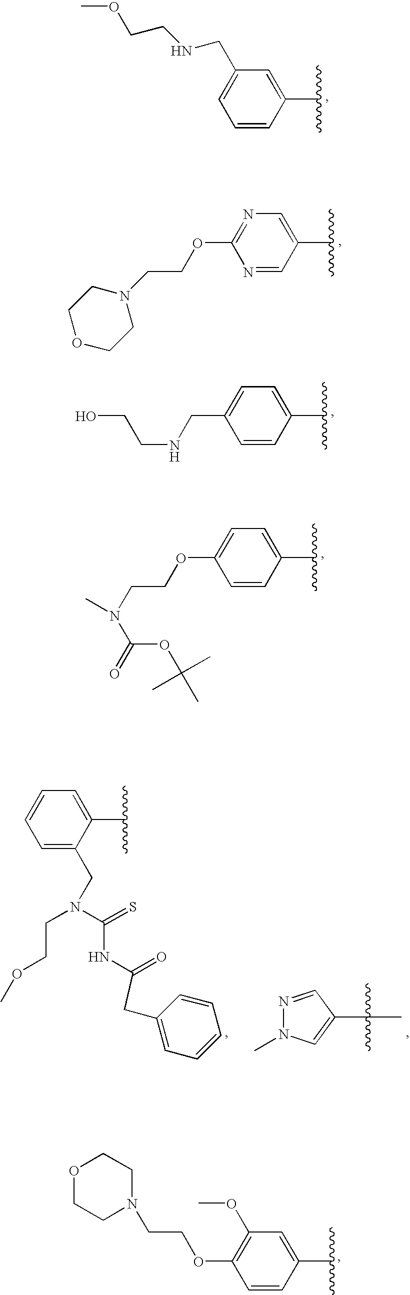

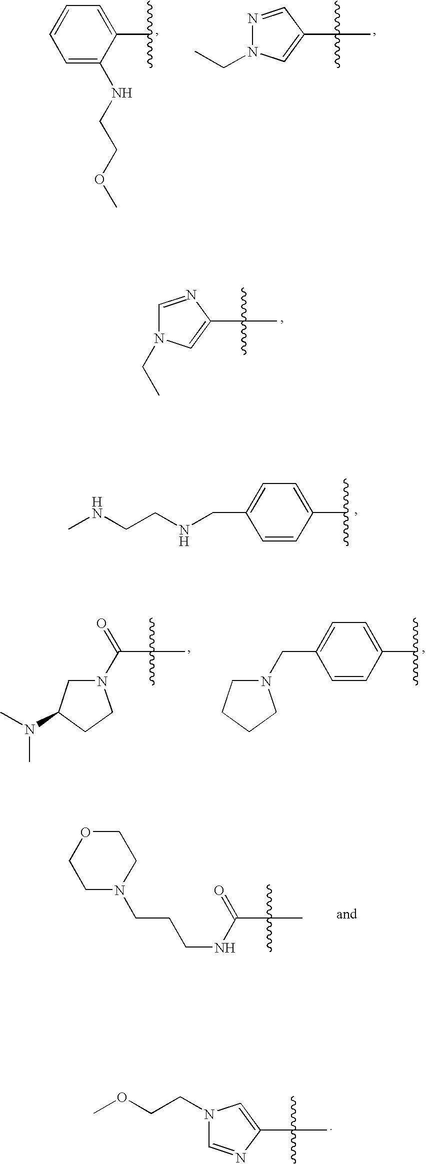

According to another preferred embodiment of the present invention, R⁷ is selected from the group consisting of phenyl and pryidyl, each of which is optionally substituted.

According to another preferred embodiment of the present invention, R⁷ groups other than —H and halogen are optionally substituted by 1 to 5 R⁸³; wherein
each R³⁸ is independently selected from halo, cyano, nitro, trifluoromethoxy, trifluoromethyl, azido, —C(O)R⁴⁰—C(O)OR⁴⁰, —OC(O)R⁴⁰, —OC(O)OR⁴⁰, —NR³⁶C(O)R³⁹, —C(O)NR³⁶R³⁹NR³⁶R³⁹, —OR—, —SO₂NR³⁶R³⁹, C₁-C₆ alkyl, —(CH₂)ⱼO(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl); —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5-10 membered heterocyclyl), —C(O)(CH₂)ₙ(5-10 membered heterocyclyl), —(CH₂)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰—(CH₂)ⱼNR³⁹(CH₂)ₙO(CH₂)ᵢOR³⁷5-(CH₂)ⱼNR³⁹(CH₂)ᵢS(O)ⱼ(C₁-C₆ alkyl), —(CH₂)ⱼNR³⁹(CH₂)ₙR³⁶—SO₂(CH₂)ₙ(C₆-C₁₀ aryl), —SO₂(CH₂)ₙ(5-10 membered heterocyclyl), —(CH₂)ₙNR³⁶R³⁹, —NR³⁷SO₂NR³⁶R³⁹, SO₂R³⁶, C₂-C₆ alkenyl, C₃-C₁₀ cycloalkyl and C₁-C₆ alkylamino, wherein j is an integer ranging from 0 to 2, n is an integer ranging from 0 to 6, i is an integer ranging from 2 to 6, the —(CH₂)ᵢ— and —(CH₂)ₙ— moieties of the foregoing R³⁸ groups optionally include a carbon-carbon double or triple bond where n is an integer between 2 and 6, and the alkyl, aryl and heterocyclyl moieties of the foregoing R³⁸ groups are optionally substituted by one or more substituents independently selected from halo, cyano, nitro, trifluoromethyl, azido, —OH, —C(O)R⁴⁰, —C(O)OR⁴⁰—OC(O)R⁴⁰, —OC(O)OR⁴⁰, —NR³⁶C(O)R³⁹, —C(O)NR³⁶R³⁹, —(CH₂)ₙNR³⁶R³⁹, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, and —(CH₂)ₙOR³⁷, wherein n is an integer ranging from 0 to 6 and i is an integer ranging from 2 to 6;
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, —(CH₂)ₙCN(CH₂)ₙOR³⁷, —(CH₂)ₙCN(CH₂)ₙR³⁷, and —(CH₂)ₙOR³⁷, wherein n is an integer ranging from 0 to 6 and i is an integer ranging from 2 to 6, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —NR³⁷R⁴¹, —C₁-C₆ alkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, wherein n is an integer ranging from 0 to 6 and i is an integer ranging from 2 to 6, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6; and
each R³⁷ and R⁴¹ is independently selected from H, OR³⁶, C₁-C₆ alkyl and C₃-C₁₀ cycloalkyl.

According to another preferred embodiment of the present invention, D is defined by the group R¹, wherein R¹ is —C≡CH or —C≡C—(CR⁴⁵R⁴⁵)ₙ—R⁴⁶; wherein
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷ (CH₂)ₙNR⁴⁷R⁴³, —(CH₂)ₙOR⁴³, —(CH₂)ₙSR⁴⁹, —(CH₂)ₙS(O)R⁴⁹, —(CH₂)ₙS(O)₂R⁴⁹, OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙC(O)R⁴⁹, —C(O)₂R⁴⁹, —(CH₂)ₙSR⁴⁹, —(CH₂)ₙS(O)R⁴⁹—(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹—(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring.

According to another preferred embodiment of the present invention,
R⁴⁶ is selected from the group consisting of —N(R⁴⁷)—C(O)—N(R⁴⁷)(R⁴¹), —N(R⁴⁷)—C(O)—(CH₂)ₙ—R⁴⁸ and —(CH₂)ₙNR⁴⁷R⁴⁸; wherein
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙS(O)₂R⁴⁹ and —(CH₂)ₙCN, or R⁴⁷ and R⁴⁸, together with the atom to which they are attached, form a 3-8 membered carbo- or hetero-cyclic ring; and
R⁵⁰ and R⁵¹ are independently selected from the group consisting of H and (C₁-C₆)alkyl, or R⁵⁰ and R⁵¹, together with the atom to which they are attached, form a 3-8 membered carbo- or hetero-cyclic ring.

According to another preferred embodiment of the present invention, R¹ is selected from the group consisting of

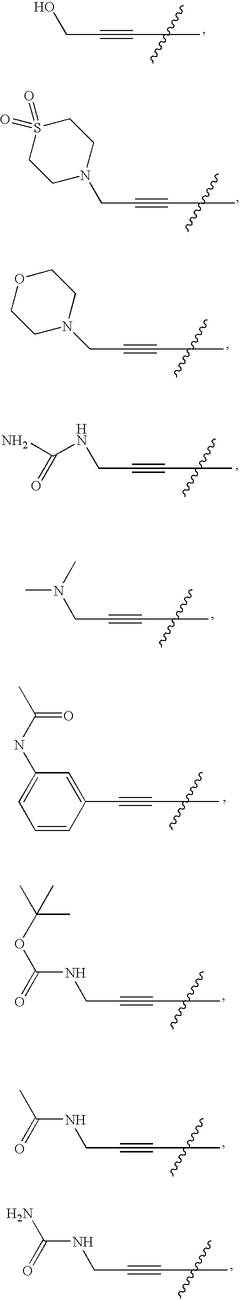

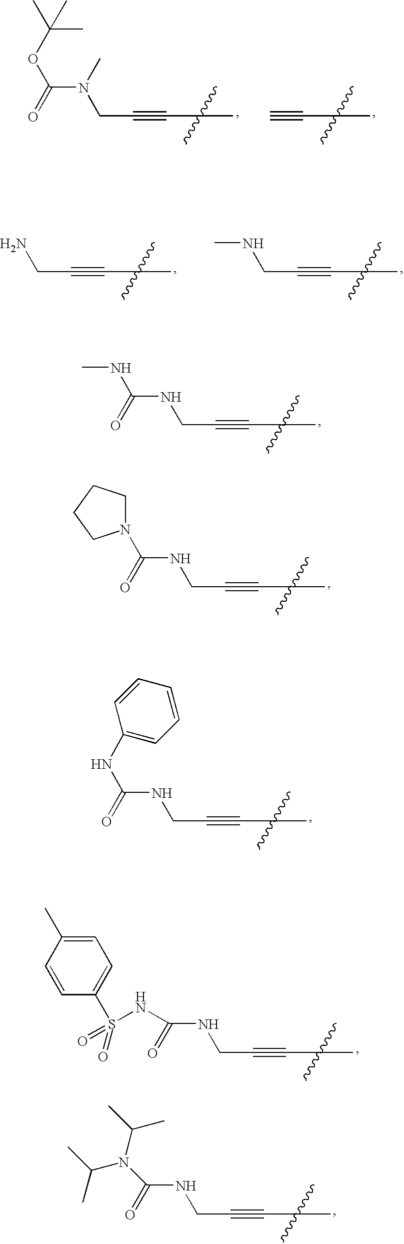

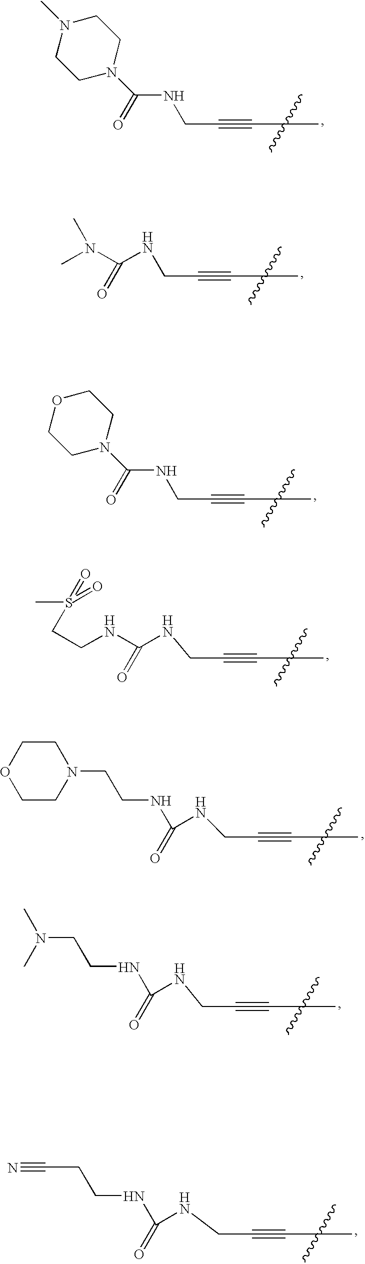

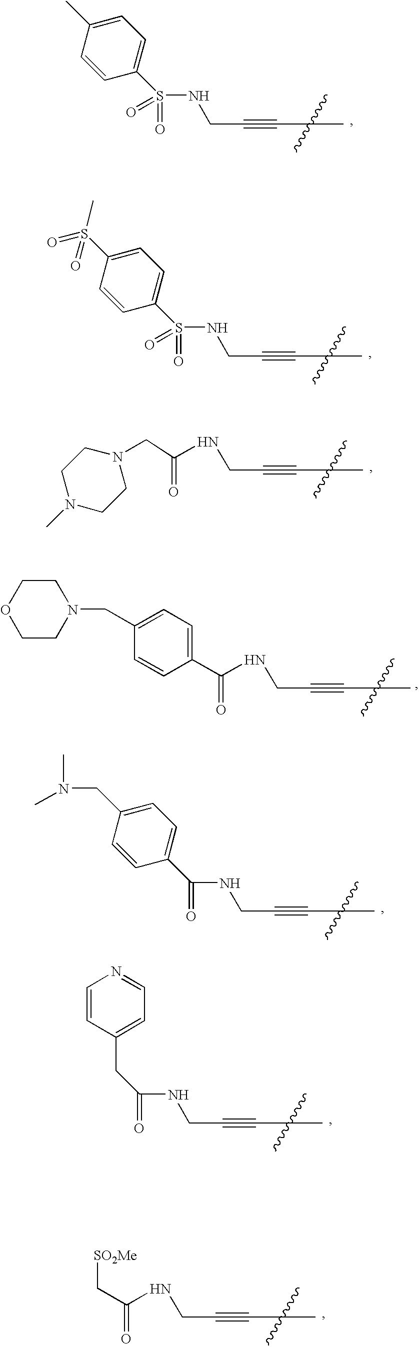

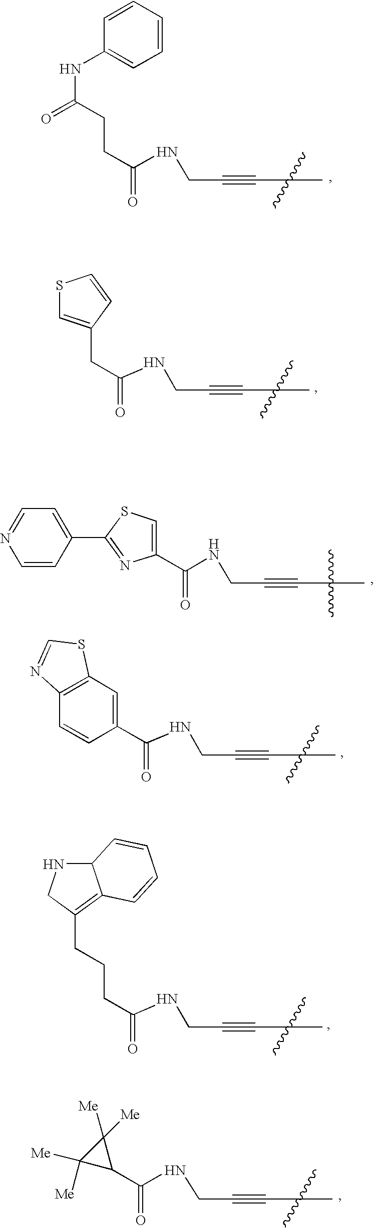

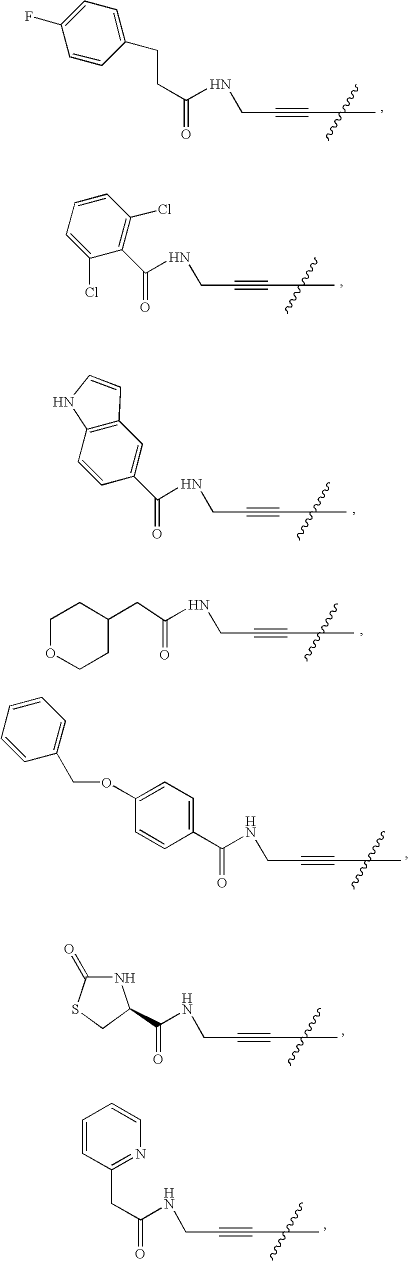

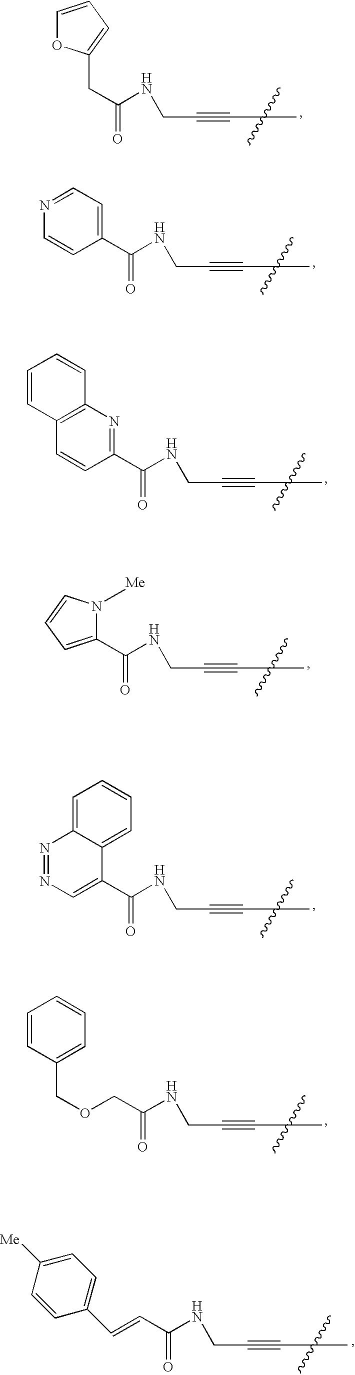

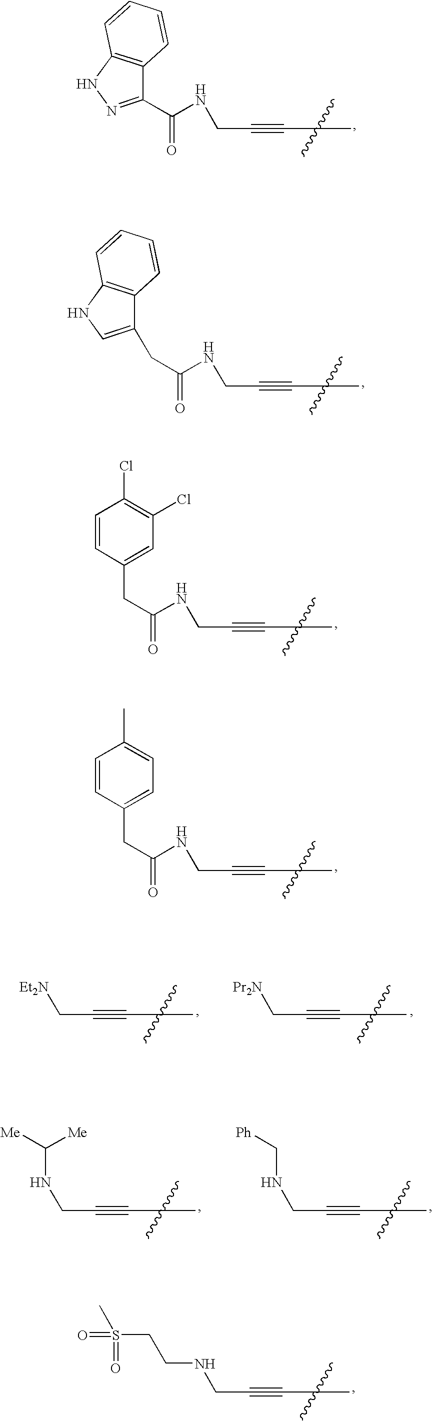

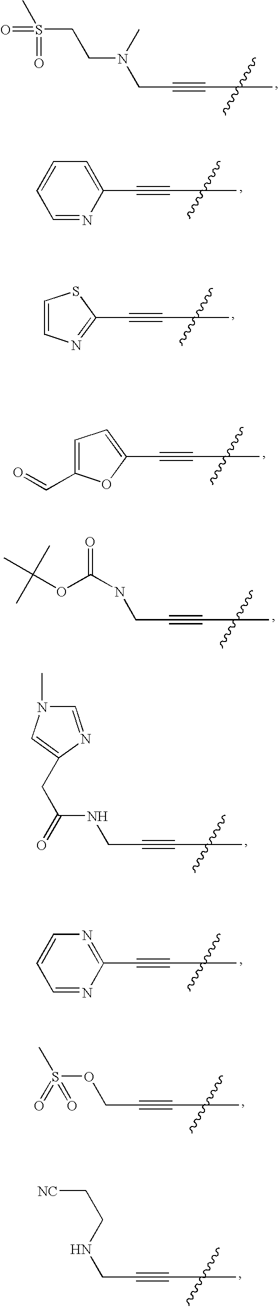

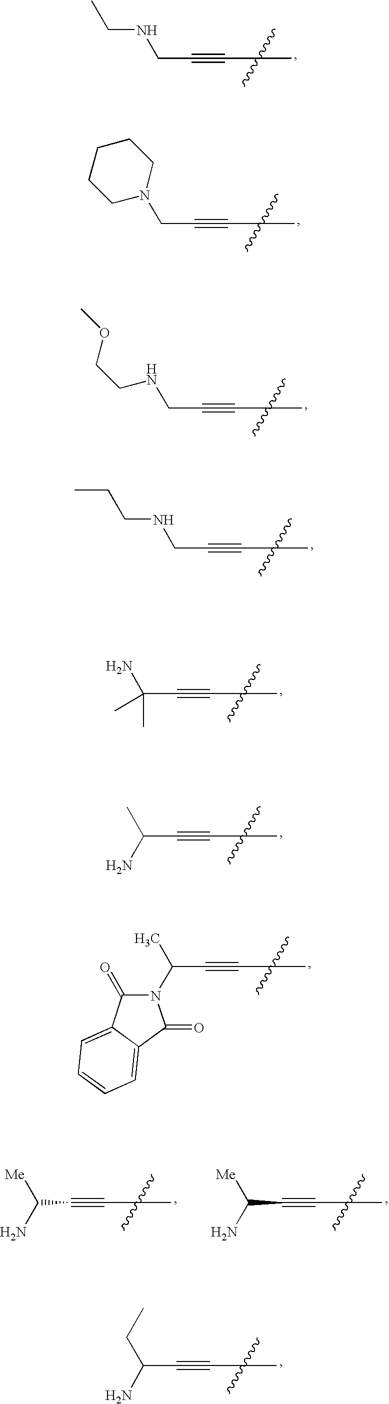

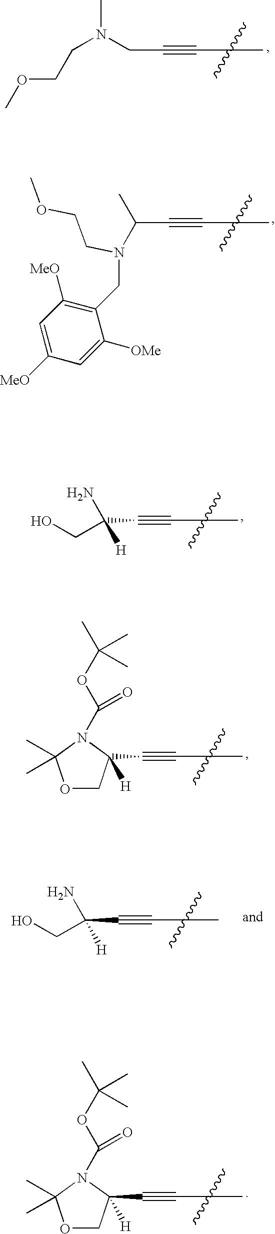

According to another preferred embodiment of the present invention, D is defined by the group R²¹, wherein R²¹ is defined by -(Z¹¹)-(Z¹²)ₘ-(Z¹³)ₘ₁, wherein
Z¹¹ is heterocyclyl, when m and m1 are 0, or heterocyclylene, when either m or ml are 1;
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆)alkyl-NR⁵³R⁵³, (C₁-C₃)alkyl, —(CH₂)_{q}OR⁵³—C(O)R⁵⁴ and —C(O)OR⁵³;
q is 0, 1, 2, 3 or 4;
each R⁵³ is independently (C₁-C₃)alkyl;
R⁵⁴ is (C₁-C₃)alkyl or N(H)R⁵³; and
R⁵⁶ is selected from the group consisting of NH₂, (C₁-C₃)alkyl and OR⁵².

According to another preferred embodiment of the present invention, Z¹¹ is a heterocyclyl and m and m1 are each 0.

According to another preferred embodiment of the present invention, Z¹¹ is a heterocyclyl and m is 0 and m1 is 0, where the heterocyclyl group is selected from the group consisting of

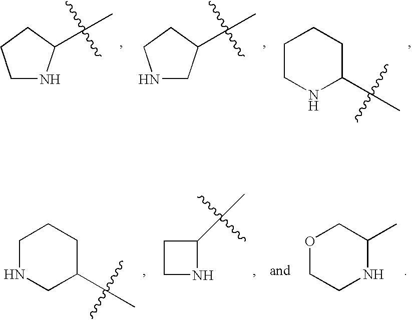

According to another preferred embodiment of the present invention, Z¹¹ is heterocyclylene, Z¹² is OC(O), m is 1, m1 is 1 and Z¹³ is heterocyclyl.

According to another preferred embodiment of the present invention, Z¹¹ is 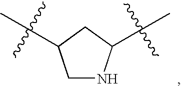
Z is OC(O), and
Z¹³ is 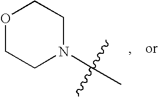
Z¹³ is N(H)R⁵², wherein R⁵² is (C₁-C₃)alkyl.

According to another preferred embodiment of the present invention, Z¹¹ is heterocyclylene, Z¹² is C(O) and m is 1, m1 is 1 and Z¹³ is (C₁-C₃)haloalkyl.

According to another preferred embodiment of the present invention, Z¹¹ is 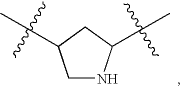
Z is C(O), and
Z¹³ is (C₁-C₃)haloalkyl, preferably —CF₃.

According to another preferred embodiment of the present invention, Z¹¹ is heterocyclylene, m is 0, m1 is 1 and Z¹³ is heterocyclyl.

According to another preferred embodiment of the present invention, Z¹¹ is 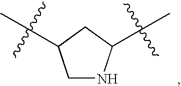
m is 0, and
Z¹³ is 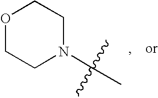
Z¹³ is (C₁-C₃)alkyl, or
Z¹³ is —OH, or
Z¹³ is —OR⁵², wherein R⁵² is (C₁-C₃)alkyl, preferably —CH₃ or
Z¹³ is halo, preferably —F, or
Z¹³ is (C₁-C₃)hydroxyalkyl, preferably —CH₃OH.

According to another preferred embodiment of the present invention, R²¹ is selected from the group consisting of

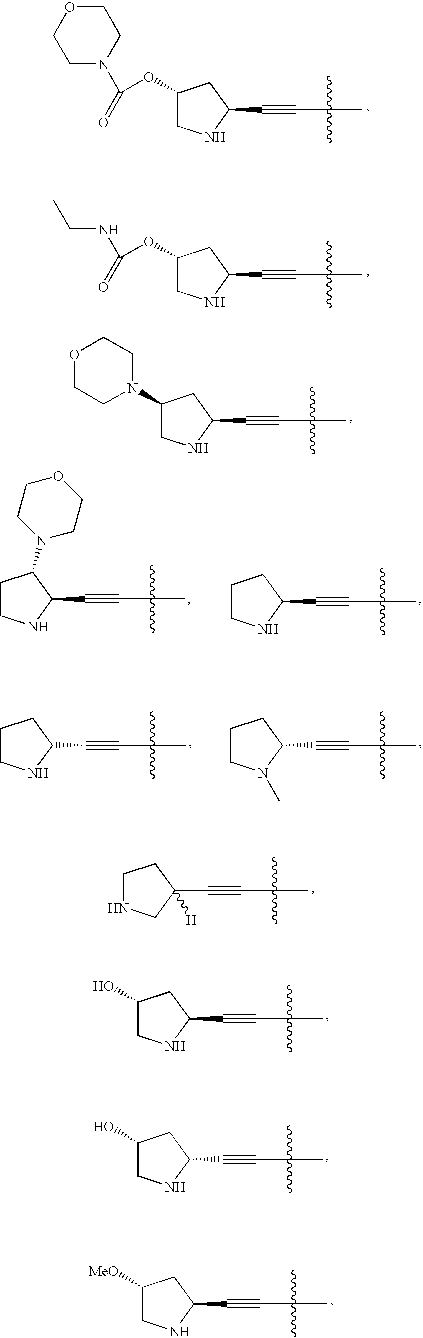

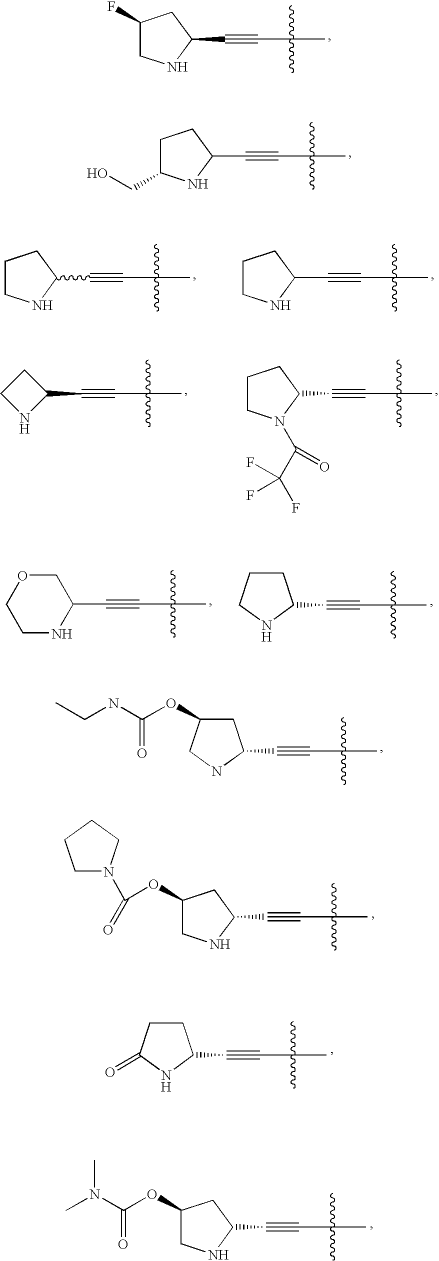

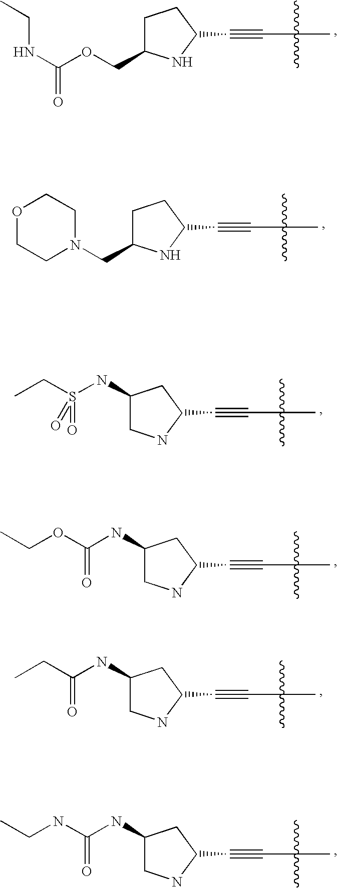

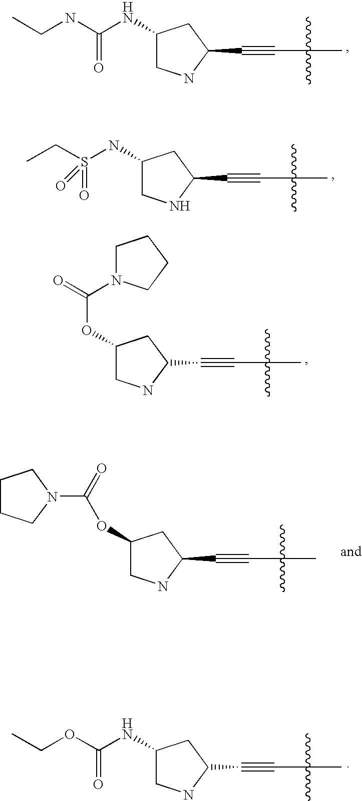

According to another preferred embodiment of the present invention, wherein D is defined by the group R²¹, the heterocyclic or heterocyclyl group is optionally substituted with a substituent selected from the group consisting of (C₁-C₆)alkyl, (C₁-C₆)alkoxy, (C₁-C₆)alkylsufanyl, (C₁-C₆)alkylsulfenyl, (C₁-C₆)alkylsulfonyl, oxo, hydroxyl, mercapto, amino optionally substituted by alkyl, carboxy, carbamoyl optionally substituted by alkyl, alkylcarboxyamide, carboxyamide, aminosulfonyl optionally substituted by alkyl, ureido, arylurea, arylthiourea, alkylurea, cycloalkylurea, sulfonylurea, nitro, cyano, halo, aryl, aralkyl, heteroaryl and (C₁-C₆)perfluoroalkyl. Such a ring may be optionally fused to one or more other “heterocyclic” ring or cycloalkyl ring. Preferred examples of “heterocyclic” moieties include, but are not limited to, tetrahydrofuranyl, pyranyl, 1,4-dioxaneyl, 1,3-dioxanyl, piperidinyl, piperazinyl, 2,4-piperazinedionyl, pyrrolidinyl, pyrrolidinon-2-yl, pyrrolidinon-3-yl, pyrrolidinon-4-yl, pyrrolidinon-5-yl, imidazolidinyl, pyrazolidinyl, morpholinyl, thiomorpholinyl, tetrahydrothiopyranyl, tetrahydrothiophenyl, and the like

According to another preferred embodiment of the present invention, wherein D is defined by the group R²¹, the heterocyclylene group is optionally substituted with substituents selected from the group consisting of (C₁-C₆)alkyl, (C₁-C₆)alkoxy, (C₁-C₆)alkylsufanyl, (C₁-C₆)alkylsulfenyl, (C₁-C₆)alkylsulfonyl, oxo, hydroxyl, mercapto, amino optionally substituted by alkyl, carboxy, carbamoyl optionally substituted by alkyl, alkylcarboxyamide, carboxyamide, aminosulfonyl optionally substituted by alkyl, ureido, arylurea, arylthiourea, alkylurea, cycloalkylurea, sulfonylurea, nitro, cyano, halo and (C₁-C₆)perfluoroalkyl, multiple degrees of substitution being allowed. Such a ring may be optionally fused to one or more benzene rings or to one or more of another “heterocyclic” rings or cycloalkyl rings. Preferred examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, pyrrolidinon-2,3-yl, pyrrolidinon-2,4-yl, pyrrolidinon-2,5-yl, pyrrolidinon-3,4-yl, pyrrolidinon-3,5-yl, pyrrolidinon-4,5-yl, morpholine-2,4-diyl, and the like.

According to another preferred embodiment of the present invention, Z is selected from the group consisting of —O—, —S—, —S(O)₀₋₂ and —NR⁵—, wherein R⁵ is selected from the group consisting of H, an optionally substituted (C₁-C₅)acyl and C₁-C₆ alkyl-O—C(O), wherein C₁-C₆ alkyl is optionally substituted.

According to another preferred embodiment of the present invention, R¹⁴ and R¹⁵ are both H, R¹⁶ is C₂-C₇ alkenyl or C₂-C₆ alkynyl and R¹⁷ is halogen, preferably fluorine.

According to another preferred embodiment of the present invention, Z is —O—.

According to another preferred embodiment of the present invention, V is selected from the group consisting of phenyl, pyrazine, pyridazine, pryimidine and pyridine, wherein each of said phenyl, pyrazine, pyridazine, pryimidine and pyridine is optionally substituted with R¹⁴, R¹⁵, R¹⁶ and R¹⁷

According to another preferred embodiment of the present invention, V is phenyl, optionally substituted with 0 to 4 R² groups.

According to another preferred embodiment of the present invention, V is phenyl, substituted with between zero and four halo.

According to another preferred embodiment of the present invention, E is —NH—.

According to another preferred embodiment of the present invention, one of R¹⁸ and R¹⁹ is —CF₃ and the other is —H.

According to another preferred embodiment of the present invention, R¹¹ and R¹² are each —H.

According to another preferred embodiment of the present invention, X is O.

According to another preferred embodiment of the present invention, R¹³ is H.

According to another preferred embodiment of the present invention, R¹¹, R¹² and R¹³ are each —H.

According to another preferred embodiment of the present invention, X is O, one of R¹⁸ and R¹⁹ is —CF₃ and the other is —H, and R¹¹, R¹² and R¹³ are each —H.

According to another preferred embodiment of the present invention, W is selected from the group consisting of 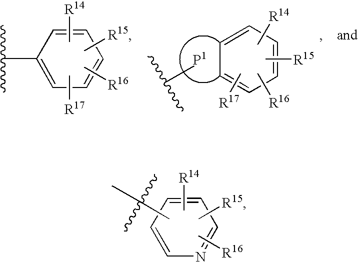
wherein P¹ is a five- to seven-membered ring, including the two shared carbon atoms of the aromatic ring to which P¹ is fused, and wherein P¹ optionally contains between one and three heteroatoms.

According to another preferred embodiment of the present invention, W is selected from the group consisting of phenyl, napthyl, 1,2,3,4-tetrahydronaphthyl, indanyl, benzodioxanyl, benzofuranyl, phenazinyl, phenothiazinyl, phenoxazinyl, tetrahydroisoquinolyl, pyrrolyl, pyrazolyl, pyrazolidinyl, imidazolyl, imidazolinyl, imidazolidinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, thiazolyl, thiazolinyl, thiazolidinyl, isothiazolyl, isothiazolidinyl, indolyl, isoindolyl, indolinyl, isoindolinyl, octahydroindolyl, octahydroisoindolyl, quinolyl, isoquinolyl, benzimidazolyl, thiadiazolyl, benzopyranyl, benzothiazolyl, benzoxazolyl, furyl, thienyl, benzothieliyl, and oxadiazolyl; each optionally substituted.

According to another preferred embodiment of the present invention, W is selected from the group consisting of phenyl, napthyl, 1,2,3,4-tetrahydronaphthyl, indanyl, benzodioxanyl, benzofuranyl, phenazinyl, phenothiazinyl, phenoxazinyl, tetrahydroisoquinolyl, pyrrolyl, pyrazolyl, pyrazolidinyl, imidazolyl, imidazolinyl, imidazolidinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, thiazolyl, thiazolinyl, thiazolidinyl, isothiazolyl, isothiazolidinyl, indolyl, isoindolyl, indolinyl, isoindolinyl, octahydroindolyl, octahydroisoindolyl, quinolyl, isoquinolyl, benzimidazolyl, thiadiazolyl, benzopyranyl, benzothiazolyl, benzoxazolyl, furyl, thienyl, benzothieliyl, and oxadiazolyl; each optionally substituted with one or more of R¹⁴, R¹⁵, R¹⁶ and R¹⁷.

According to another preferred embodiment of the present invention, W is phenyl, optionally substituted.

According to another preferred embodiment of the present invention, W is phenyl, optionally substituted with one or more of R¹⁴, R¹⁵, R¹⁶ and R¹⁷.

According to another preferred embodiment of the present invention, W is substituted by a halogen and either an alkenyl or alkynyl.

According to another preferred embodiment of the present invention W is phenyl.

According to another preferred embodiment of the present invention W is phenyl substituted by a halogen and either an alkenyl or alkynyl.

According to another preferred embodiment of the present invention, U¹ is selected from the group consisting of 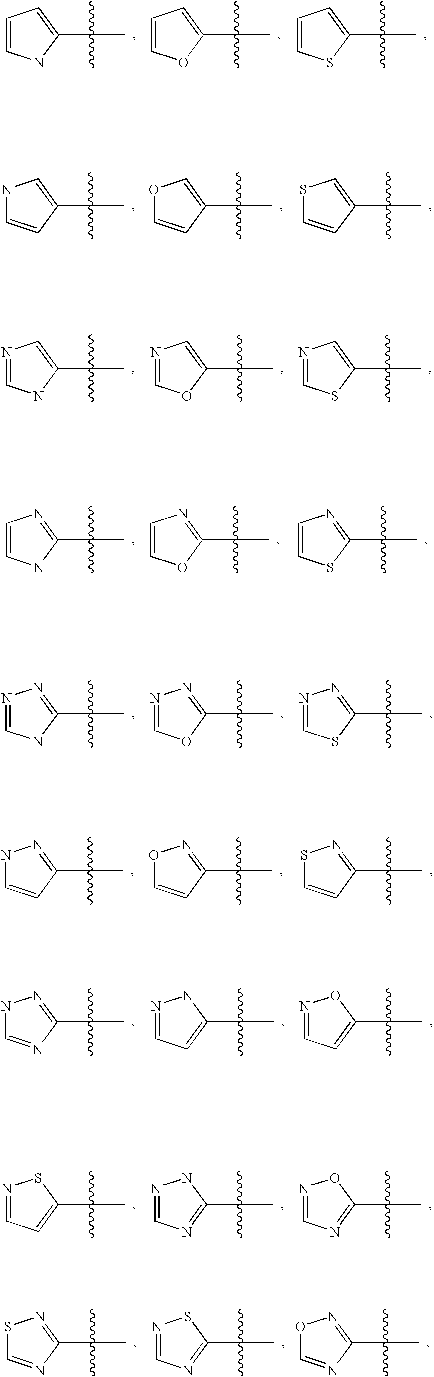 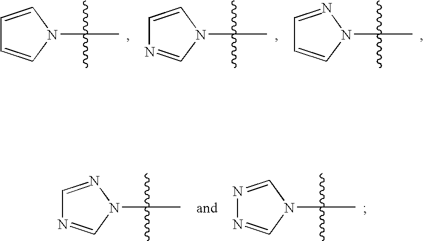
wherein the members of said group are optionally substituted.

According to another preferred embodiment of the present invention, U¹ is selected from the group consisting of 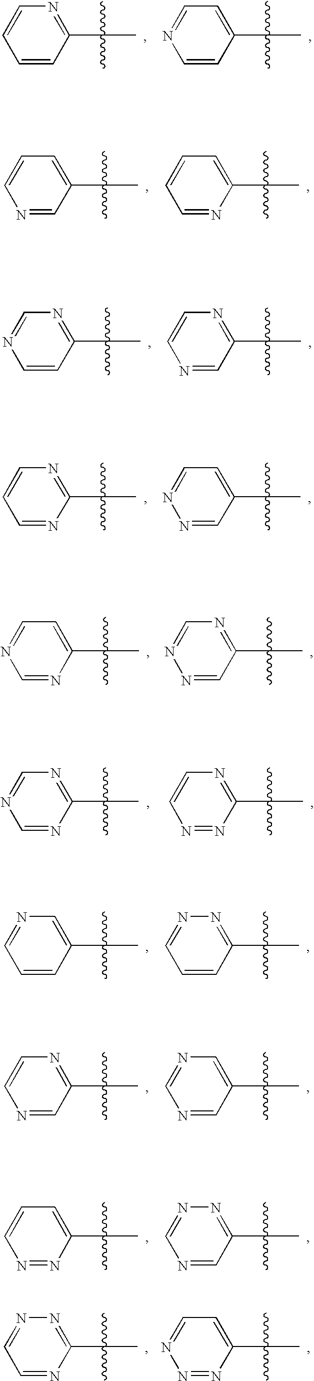 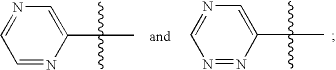
wherein the members of said group are optionally substituted.

Another embodiment of the present invention provides a composition comprising a therapeutically effective amount of a compound, or racemic mixtures, diastereomers and enantiomers thereof, according to any embodiment or preferred embodiment thereof of the present invention, or an N-oxide, hydrate, solvate, pharmaceutically acceptable salt, prodrug or complex thereof, together with a pharmaceutically acceptable carrier, excipient or diluent.

A further aspect of the present invention provides a method of inhibiting receptor type tyrosine kinase signaling, preferably VEGF receptor signaling and HGF receptor signaling, the method comprising contacting the receptor with a compound, or racemic mixtures, diastereomers and enantiomers thereof, according to any embodiment or preferred embodiment thereof of the present invention, or an N-oxide, hydrate, solvate, pharmaceutically acceptable salt, prodrug or complex thereof, or with a composition according to the present invention. Inhibition of receptor type tyrosine kinase activity, preferably VEGF and HGF receptor signaling can be in a cell or a multicellular organism. If in a multicellular organism, the method according to this aspect of the invention comprises administering to the organism a compound, or racemic mixtures, diastereomers and enantiomers thereof, according to any embodiment or preferred embodiment of the present invention, or an N-oxide, hydrate, solvate, pharmaceutically acceptable salt, prodrug or complex thereof, or a composition according to the present invention. Preferably the organism is a mammal, more preferably a human.

The data presented herein demonstrate the inhibitory effects of the kinase inhibitors of the invention. These data lead one to reasonably expect that the compounds of the invention are useful not only for inhibition of protein tyrosine kinase activity, or preferably VEGF receptor signaling and HGF receptor signaling, but also as therapeutic agents for the treatment of proliferative diseases, including cancer and tumor growth.

Preferred compounds according to the invention include those described in the examples below. Compounds were named using Chemdraw Ultra version 10.0 or version 8.0.3, which are available through Cambridgesoft.com, or were derived therefrom.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

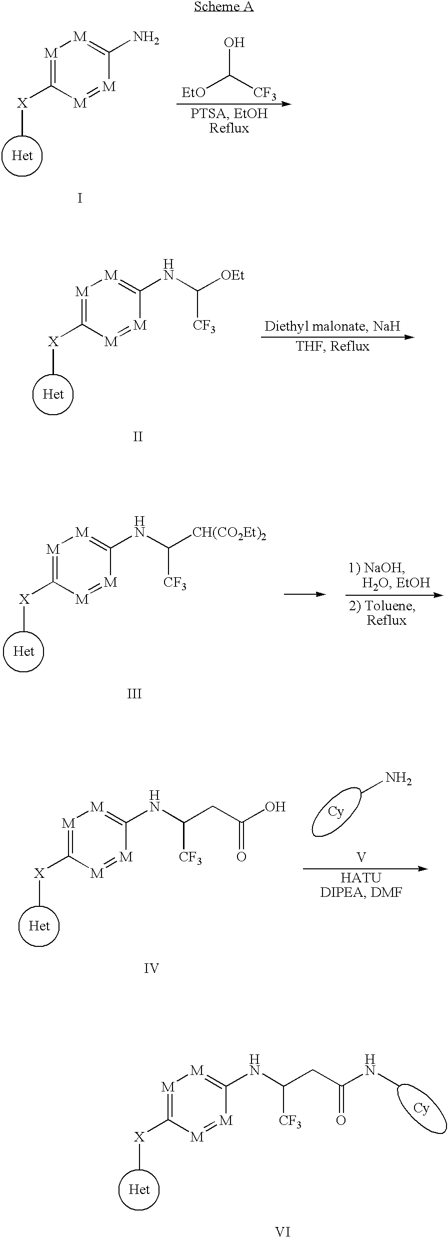

### Synthesis of 4,4,4-trifluoro-N-cyclyl-3-(amino)butanamides (VI)

4,4,4-Trifluoro-N-cyclyl-3-(amino)butanamides of the general formula VI may be obtained via a short reaction sequence starting from the amines I which represent appropriately substituted various scaffolds suitable for the synthesis of kinase inhibitors or other compounds of pharmaceutical interest. Amines I upon treatment with trifluoroacetaldehyde ethyl hemiacetal under acidic conditions (e.g. in the presence of 4-toluenesulfonic acid) in polar solvents such as ethanol are transformed into N-(1-ethoxy-2,2,2-trifluoroethyl)amines of the general structure II. Compounds II reacting with malonates under basic conditions form 2-(2,2,2-trifluoro-1-(amino)ethyl)malonates such as III. The amino di-esters III undergo alkaline hydrolysis to form the intermediate malonic acids (not shown in the scheme A), which are further decarboxylated, to afford 4,4,4-trifluoro-3-(amino)butanoic acids IV. Acids IV are coupled to different primary or secondary amines V using standard techniques, to produce title compounds VI.

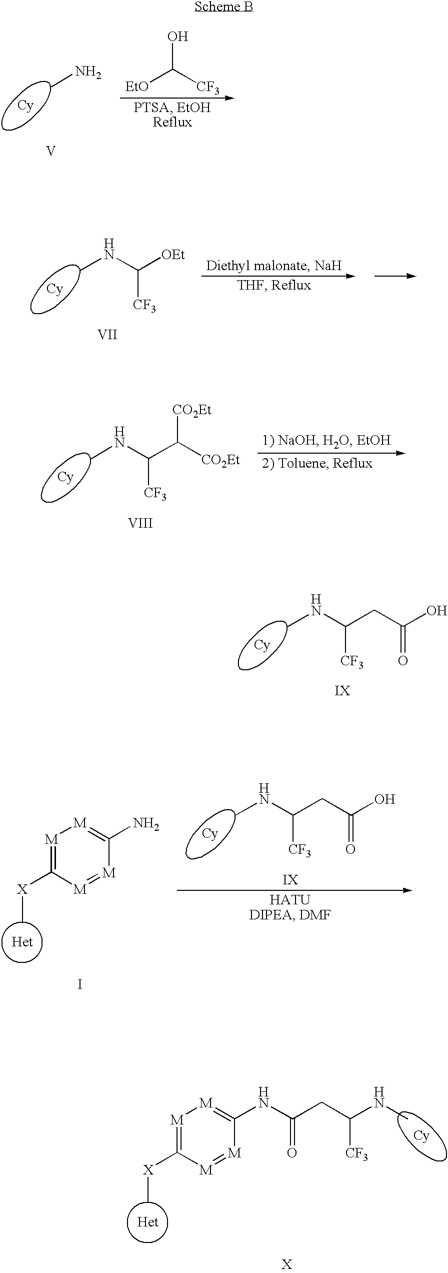

### Synthesis of 4,4,4-trifluoro-N-3-(cycylyamino)butanamides (X)

4,4,4-Trifluoro-N-3-(cyclylamino)butanamides of the general formula X may be obtained via a similar short reaction sequence as in Scheme A using the same sets of amines I and amines V. Amines V upon treatment with trifluoroacetaldehyde ethyl hemiacetal under acidic conditions (e.g. in the presence of 4-toluenesulfonic acid) in polar solvents such as ethanol are transformed into N-(1-ethoxy-2,2,2-trifluoroethyl)cyclylamines of the general structure VII. Compounds VII reacting with malonates under basic conditions form diethyl 2-(2,2,2-trifluoro-1-(cyclylamino)ethyl)malonates such as VIII. The amino di-esters VIII undergo alkaline hydrolysis to form the intermediate malonic acids (not shown in the scheme B), which are further decarboxylated, to afford 4,4,4-trifluoro-3-(cyclylamino)butanoic acids IX. Acids IX are coupled to various amines of the general structure I, using standard techniques, to produce title compounds X.

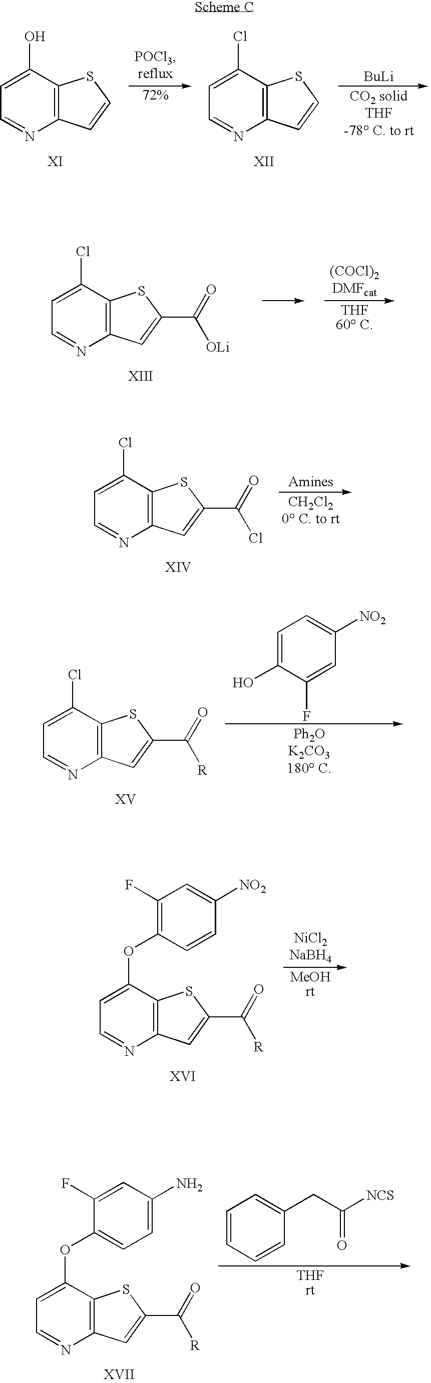

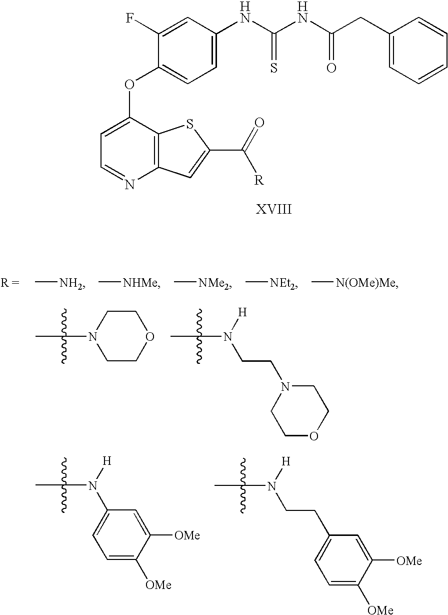

Thieno[3,2-b]pyridine based compounds of formula XVIII may be prepared according to the procedures illustrated in the scheme C. Thus, thieno[3,2-b]pyridine-7-ol (XI) upon treatment with POCl₃ is converted to the chloride XII. Treatment of this material with a strong base such as n-BuLi followed by an addition of carbon dioxide affords the carboxylate XIII which is used without purification in the next step, providing the acyl chloride XIV upon its reaction with oxalyl chloride. The acyl chloride XIV is used for the next step without further purification as well: upon its reaction with different primary and secondary amines the compound XIV is converted to a variety of primary and secondary amides XV which can further be derivatized via a substitution of the chlorine atom in the pyridine ring.

Thus, XV reacting with 2-fluoro-4-nitrophenol in a high boiling point solvent, such as diphenyl ether in the presence of a base such as potassium carbonate, produced the nitro derivatives XVI which are then reduced to the amines XVII upon treatment with a mixture NiCl₂/NaBH₄. The amines XVII also are used for the next step without further purification, and upon treatment with benzyl isothiocyanate afford the phenylacetylthioureas XVIII bearing the amido-substituents such as the ones shown in the scheme C.

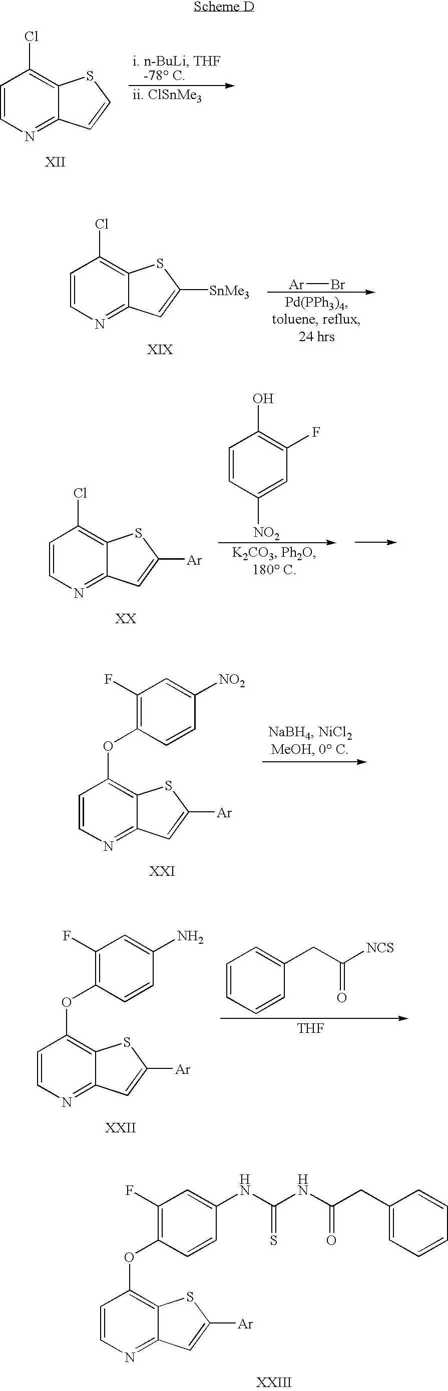

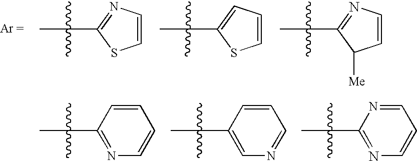

Thieno[3,2-b]pyridine based phenylacetylureas of formula XXIII bearing heteroaryl substituents instead of the amido moieties may be prepared according to the procedures illustrated in the scheme D. Thus, treatment of the chloride XII with a strong base such as n-BuLi followed by an addition of trimethyltin chloride affords the trimethylstannyl derivative XIX. This material reacting with different aryl bromides in the presence of a Pd-catalyst produces aryl-substituted thienopyridines XX which can further be derivatized via a substitution of the chlorine atom in the pyridine ring.

Thus, XX reacting with 2-fluoro-4-nitrophenol in a high boiling point solvent, such as diphenyl ether in the presence of a base such as potassium carbonate, produced the nitro derivatives XXI which are then reduced to the amines XXII upon treatment with a mixture NiCl₂/NaBH₄. The amines XXII are used for the next step without further purification, and upon treatment with benzyl isothiocyanate afford the phenylacetylthio-ureas XXIII bearing the heteroaryl substituents such as the ones shown in the scheme D.

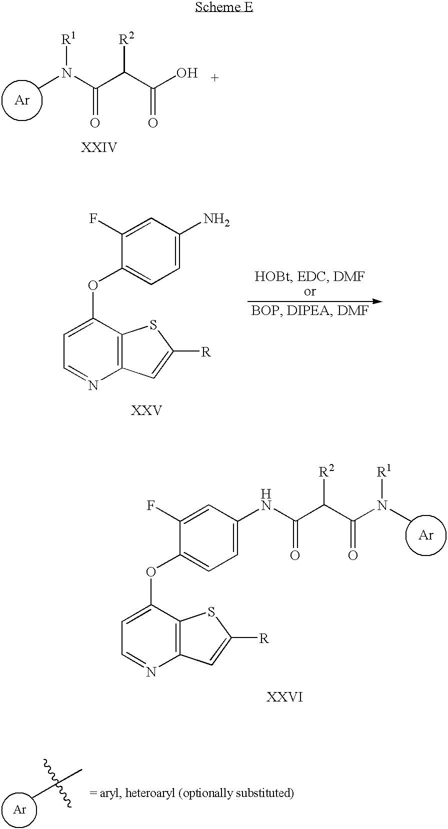

Thieno[3,2-b]pyridine based compounds of formula XXVI may be prepared according to a general procedure shown in the scheme E, via an amide coupling reaction between N-aryl(heteroaryl)-malonamic acids [3-oxo-3-(arylamino)- or 3-oxo-3-(heteroarylamino)-propanoic acids] (XXIV) and thieno[3,2-b]pyridine derivatives bearing an amino-group (XXV).

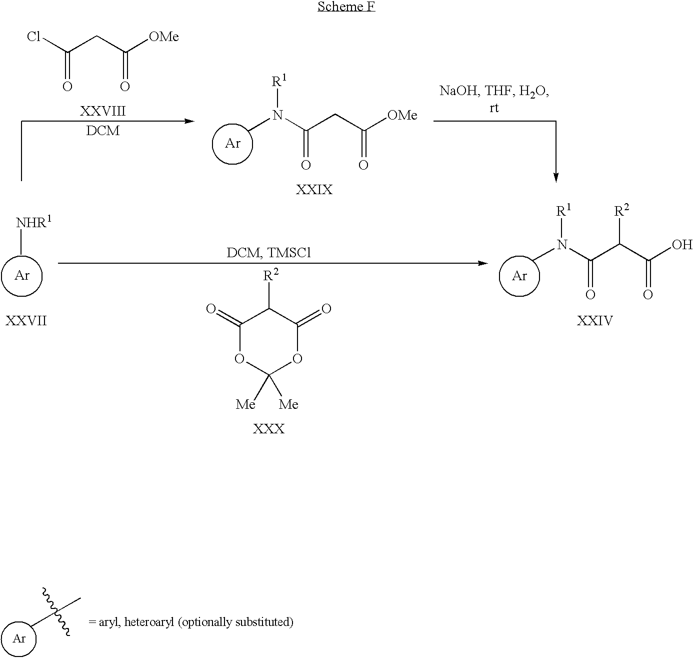

Acids XXIV typically could be prepared according to the scheme F by reacting the amines XXVII either with 3-chloro-3-oxopropanoate (XXVIII) via the intermediate amino esters XXIX which have to be hydrolyzed (two-step procedure), or with 2,2-dimethyl-[1,3]dioxane-4,6-dione (Meldrum's acid) (XXX) in the presence of TMSCl, to form the desired acid XXIV in one step.

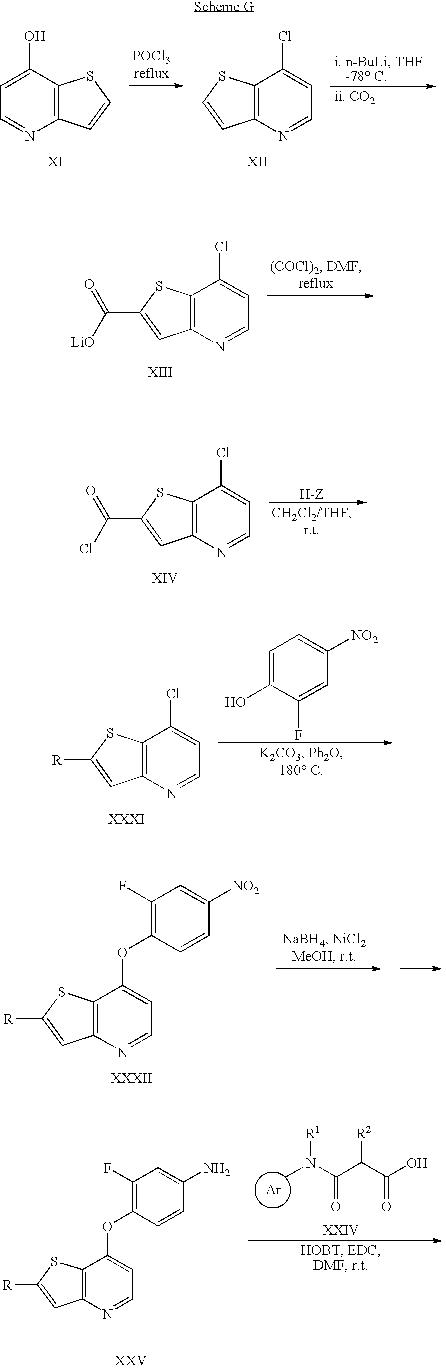

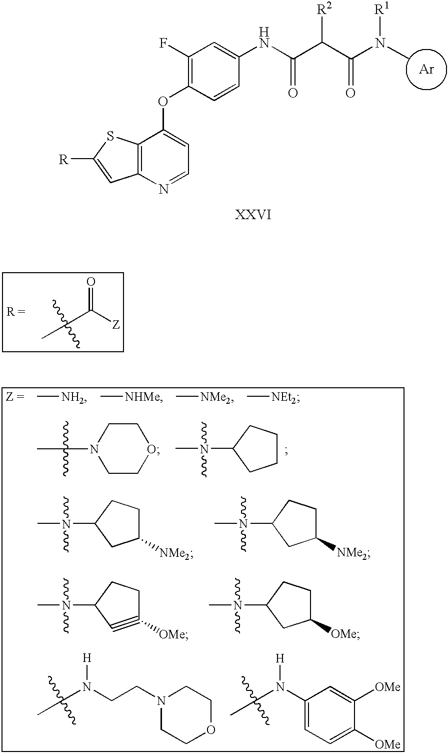

Thieno[3,2-b]pyridine derivatives bearing an amino-group (XXV) could be prepared in different ways depending on the nature of the substituent R on the thiophene ring of the thienopyridine bicyclic ring system. For example, when R is an amide moiety synthetic sequence shown in the Scheme G, can be employed.

Thus, thieno[3,2-b]pyridine-7-ol (XI) reacting with POCl₃ is converted to the chloride XII. Treatment of this material with a strong base such as n-BuLi followed by an addition of carbon dioxide affords the carboxylate XIII which is used without purification in the next step, providing the acyl chloride XIV upon its reaction with oxalyl chloride. The acyl chloride XIV is used for the next step without further purification as well: upon its reaction with different primary or secondary amines the compound XIV is converted to a variety of primary and secondary amides XXXI which can further be derivatized via a substitution of the chlorine atom of the pyridine ring. Thus, XXXI reacting with 2-fluoro-4-nitrophenol in a high boiling point solvent, such as diphenyl ether in the presence of a base such as potassium carbonate, produced the nitro derivatives XXXII which are then reduced to the amines XXV upon treatment with a mixture NiCl₂/NaBH₄. The amines XXV (could be used for the next step without further purification) upon treatment with N-aryl(heteroaryl)-malonamic acids (XXIV) afford malonamides XXVI bearing the amido-substituents on the thiophene ring such as the ones shown in the scheme G.

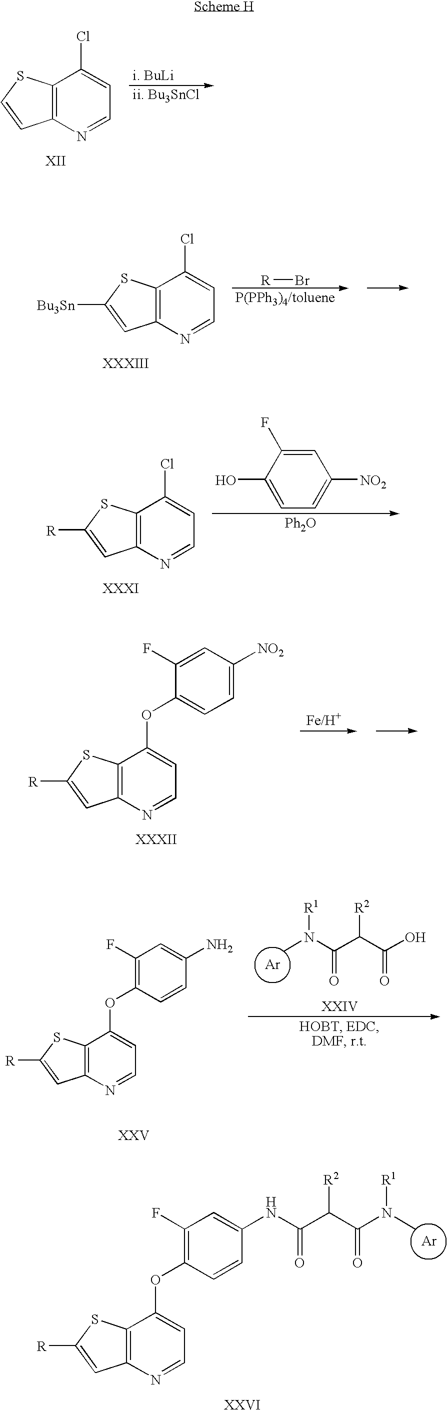

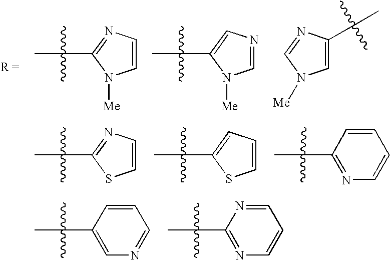

Thieno[3,2-b]pyridine based malonamides of formula XXVI bearing heteroaryl substituents instead of the amido moieties may be prepared using procedures illustrated in the Scheme H. Thus, treatment of the chloride XXXI with a strong base such as n-BuLi followed by an addition tributyltin chloride affords the tributylstannyl derivative XXXIII. This material reacting with different heteroaryl bromides in the presence of a Pd-catalyst (Stille coupling reaction) produces heteroaryl-substituted thienopyridines XXXI (R=heteroaryl) which can further be derivatized via a substitution of the chlorine atom of the pyridine ring of the thienopyridine ring system.

Thus, XXXI reacting with 2-fluoro-4-nitrophenol in a high boiling point solvent, such as diphenyl ether in the presence of a base such as potassium carbonate, produced the nitro derivatives XXXII which are then reduced to the amines XXV upon treatment with iron in an acidic medium. The amines XXV (could be used for the next step without further purification) upon treatment with N-aryl(heteroaryl)-malonamic acids (XXIV) afford malonamides XXVI bearing heteroaryl substituents on the thiophene ring such as the ones shown in the Scheme H.

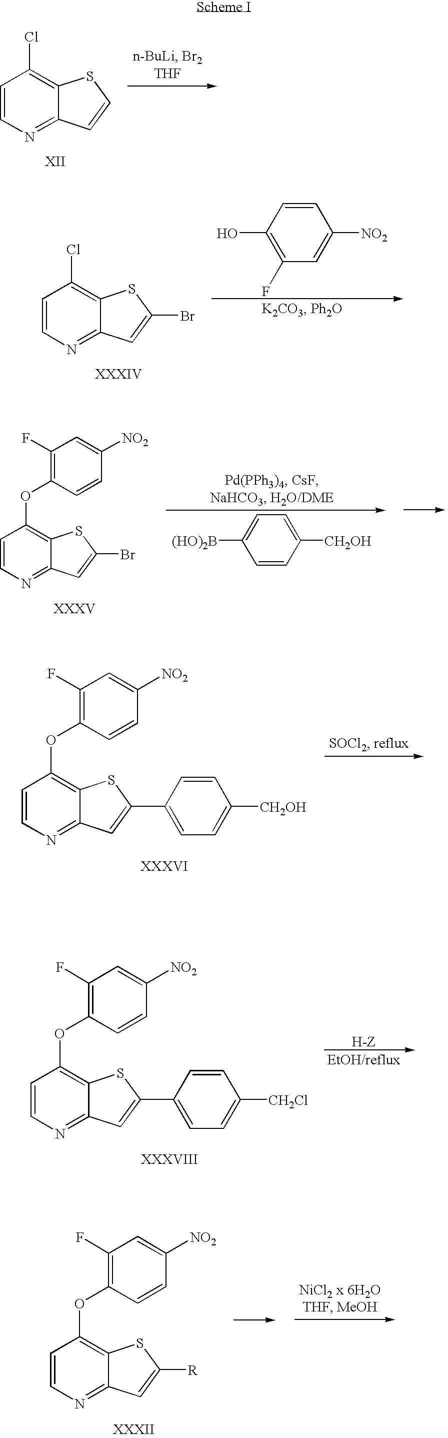

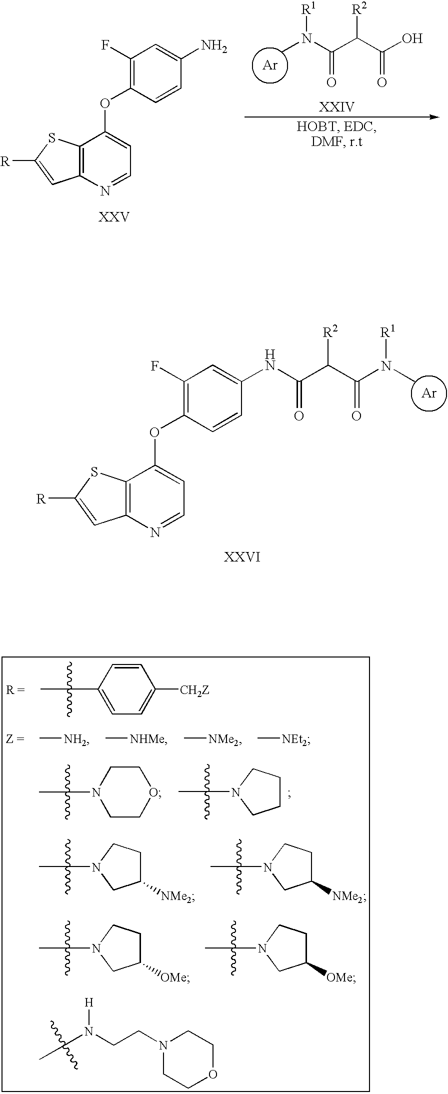

Thieno[3,2-b]pyridine based malonamides of formula XXVI bearing aryl substituents on the thiophene ring, particularly aryl substituents with basic moieties, may be prepared using procedures illustrated in the Scheme I. Thus, treatment of the chloride XII with a strong base such as n-BuLi followed by bromination (for instance, with elemental bromine) affords the bromide XXXIV. This material reacting with 2-fluoro-4-nitrophenol in a high boiling point solvent, such as diphenyl ether in the presence of a base such as potassium carbonate, produced the nitro derivative XXXV which underwent a reaction with 4-(hydroxymethyl)phenylboronic acid in the presence of a base and a Pd-catalyst (Suzuki coupling reaction) to provide aryl-substituted derivative XXXVI with a free hydroxyl group. The hydroxyl group was replaced by a halogen (for example, chloride using the thionyl chloride) to form the compound XXXVII which upon treatment with secondary and primary amines was converted into a variety of aryl-substituted compounds XXXII (R=substituted aryl). The nitro group of these basic entities was reduced with NaBH₄/NiCl₂ to form the amines XXV. These intermediates (could be used for the next step without further purification) upon treatment with N-aryl(heteroaryl)-malonamic acids (XXIV) afford malonamides XXVI bearing aryl substituents with basic moieties, attached to the thiophene ring such as the ones shown in the Scheme I.

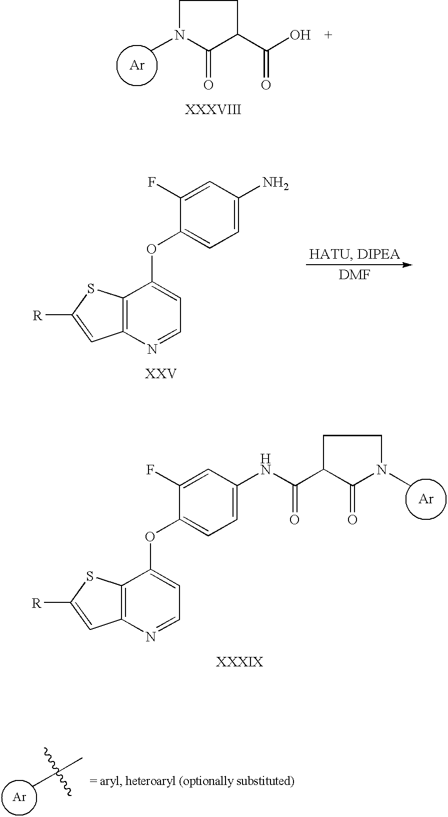

Thieno[3,2-b]pyridine based compounds of formula XXXIX may be prepared according to a general procedure shown in the scheme J, via an amide coupling reaction between 2-oxo-1-aryl(heteroaryl)pyrrolidine-3-carboxylic acids (XXXVIII) and thieno[3,2-b]pyridine derivatives bearing an amino-group (XXV). Acids XXXVIII could be prepared following the literature procedure [S. Danishefsky, R. K. Singh. JACS, 1975, 97, 3239-3241] or purchased if commercially available.

### PARTICULAR EXAMPLES

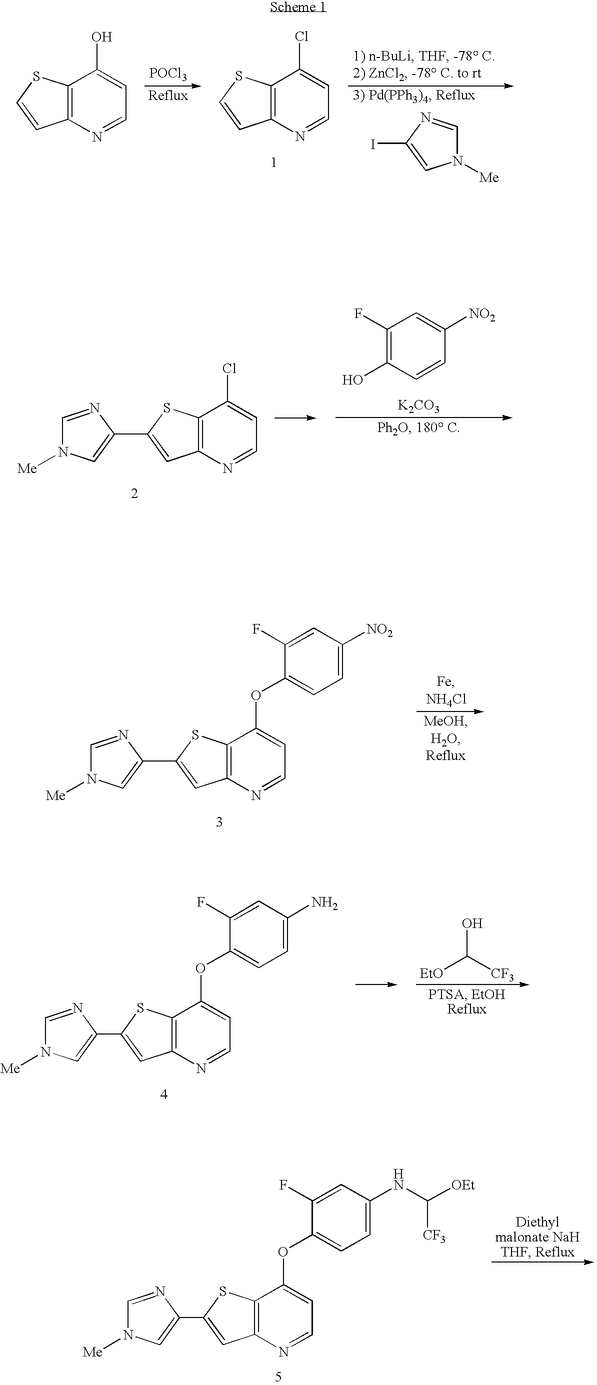

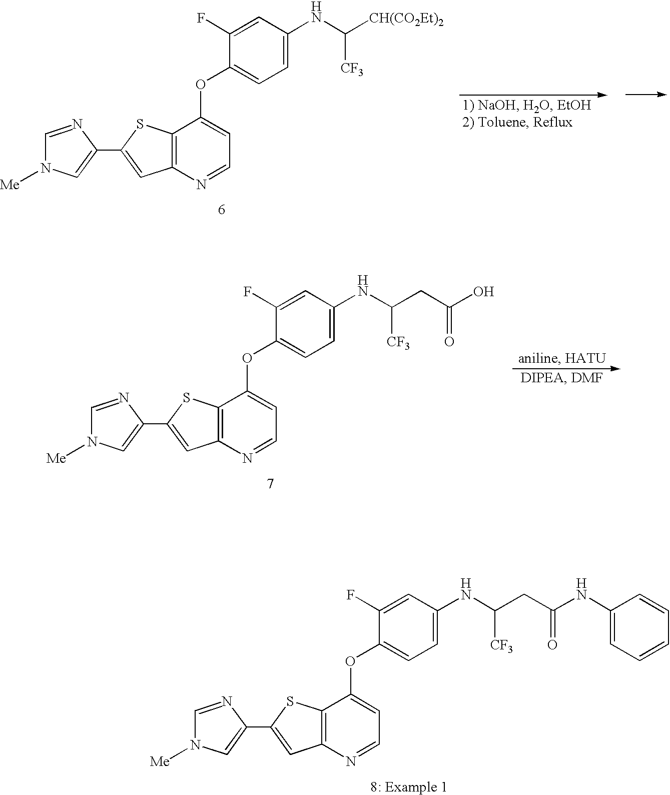

### Example 1

### 4,4,4-Trifluoro-3-(3-fluoro-4-(2-(1-methyl-1H-imidazol-4-yl)thieno[3,2-b]pyridin-7-yloxy)phenylamino)-N-phenylbutanamide (8)

### Step 1: 7-Chlorothieno[3,2-b]pyridine (1)

A stirred suspension of thieno[3,2-b]pyridin-7-ol (5.0 g, 33.1 mmol) in POCl₃ (15 mL) was heated to 105° C. in an oil bath for 4 h. The resultant solution was cooled to room temperature and the POCl₃ was removed under reduced pressure. The residue was cooled in an ice/water bath and cold water was added. The water was made basic with concentrated NH₄OH solution and extracted with EtOAc. The organic extract was dried over anhydrous sodium sulfate and concentrated to produce an oil which was purified by column chromatography (eluent EtOAc-hexane, 1:4) to afford the title compound 1 as a brown solid (4.5 g, 72% yield). ¹H NMR (400 MHz, CDCl₃) δ (ppm): 8.60 (d, J=4.9 Hz, 1H), 7.80 (d, J=5.5 Hz, 1H), 7.60 (d, J=5.5 Hz, 1H), 7.30 (d, J=4.9 Hz, 1H).

### Step 2. 7-Chloro-2-(1-methyl-1H-imidazol-4-yl)thieno[3,2-b]pyridine (2)

To a solution of chloride 1 (2.45 g, 14.4 mmol) in THF (48 mL) at −78° C. was slowly added n-BuLi (2.5M in hexane, 7.2 mL, 18.0 mmol). The reaction mixture was stirred for one hour at −78° C. followed by slow addition of ZnCl₂ (0.5M in THF, 36 mL, 18.0 mmol). The reaction mixture was allowed to warm to room temperature and stirring was continued for one hour.

A solution of 4-iodo-1-methyl-1H-imidazole (1.50 g, 7.2 mmol) [Tet. Lett. 2004, 45, 5529] in THF (5 mL) and tetrakis(triphenylphosphine) palladium (0) (0.83 g, 0.72 mmol) were added to the reaction mixture which was heated to reflux for 1 hour, cooled to room temperature, diluted with aqueous ammonium hydroxide and, finally neutralized with a 1N HCl solution. The acidic solution was extracted with dichloromethane, the extract was washed with water and brine, dried over anhydrous magnesium sulfate, filtered and evaporated under reduced pressure. The residue was purified by flash chromatography (eluents DCM, then DCM-MeOH, 97:3) to afford title compound 2 (1.45 g, 81% yield) as a yellow solid. MS (m/z): 263.9, 265.9 (M+H).

### Step 3. 7-(2-Fluoro-4-nitrophenoxy)-2-(1-methyl-1H-imidazol-4-yl)thieno[3,2-b]pyridine (3)

To a suspension of 2 (377 mg, 1.51 mmol) in Ph₂O (2 mL) was added 2-fluoro-4-nitrophenol (474 mg, 3.02 mmol) and potassium carbonate (626 mg, 4.53 mmol). The reaction mixture was heated at 180° C. for 2 h, cooled to room temperature, diluted with dichloromethane and MeOH and filtered. The filtrate was collected and concentrated under reduced pressure. The residue was adsorbed on silica gel and purified by column chromatography (eluent EtOAc-hexane 4:1). The product was triturated with a mixture of EtOAc and hexane to afford 3 (460 mg, 82% yield) as a yellow solid. ¹H NMR (400 MHz, DMSO-d₆) δ (ppm): 8.53 (d, J=5.5 Hz, 1H), 8.48 (dd, J=10.0, 2.6 Hz, 1H), 8.20 (qd, J=9.2, 1.4 Hz, 1H), 7.89 (d, J=1.2 Hz, 1H), 7.74 (s, 1H), 7.73 (d, J=1.0 Hz, 1H), 7.69 (dd, J=8.8, 8.2 Hz, 1H), 6.87 (d, J=5.5 Hz, 1H), 3.73 (s, 3H). MS (m/z): 371.0 (M+H).

### Step 4. 3-Fluoro-4-(2-(1-methyl-1H-imidazol-4-yl)thieno[3,2-b]pyridin-7-yloxy)benzenamine (4)

To a stirred suspension of 3 (460 mg, 1.24 mmol) in MeOH (12 mL) and water (6 mL) under nitrogen were successively added ammonium chloride (57 mg, 1.07 mmol) and iron (reduced powder, 589 mg, 10.54 mmol). The mixture was heated to reflux for 40 min., cooled to room temperature, filtered through celite, rinsed with EtOAc and the solvents were removed under reduced pressure. The residue was purified by column chromatography on silica gel (eluent MeOH-dichloromethane 5:95). The product was triturated with a mixture of EtOAc and hexane, to afford 4 (322 mg, 0.95 mmol, 76% yield) as a yellow solid. ¹H NMR (400 MHz, DMSO-d₆) δ (ppm): 8.40 (d, J=5.5 Hz, 1H), 7.85 (d, J=1.2 Hz, 1H), 7.71 (d, J=1.2 Hz, 1H), 7.65 (s, 1H), 7.10 (t, J=9.2 Hz, 1H), 6.53 (dd, J=13.2, 2.4 Hz, 1H), 6.50 (d, J=6.1 Hz, 1H), 6.44 (dd, J=8.8, 3.2 Hz, 1H), 5.55 (s, 2H), 3.72 (s, 3H).

### Step 5. N-(1-Ethoxy-2,2,2-trifluoroethyl)-3-fluoro-4-(2-(1-methyl-1H-imidazol-4-yl)thieno[3,2-b]pyridin-7-yloxy)benzenamine (5)

A mixture of 4 (500 mg, 1.47 mmol), trifluoroacetaldehyde ethyl hemiacetal (0.35 mL, 2.94 mmol) and 4-toluenesulfonic acid monohydrate (280 mg, 1.47 mmol) in ethanol (25 mL) was heated to reflux for 48 h. The reaction mixture was cooled, concentrated and the residue was purified by column chromatography on silica gel (eluent MeOH-dichloromethane 5:95 to 8:92) to afford the title compound 5 (470 mg, 68% yield). ¹H NMR (400 MHz, DMSO-d₆) δ (ppm): 8.42 (d, J=5.5 Hz, 1H), 7.85 (d, J=1.2 Hz, 1H), 7.72 (d, J=0.8 Hz, 1H), 7.67 (s, 1H), 7.29 (t, J=9.2 Hz, 1H), 7.08-7.02 (m, 2H), 3.86 (dd, J=9.2, 2.0 Hz, 1H), 6.52 (d, J=5.5 Hz, 1H), 5.68 (qd, J=10.4, 5.2 Hz, 1H), 3.72 (s, 3H), 3.76-3.59 (m, 2H), 1.15 (t, J=7.0 Hz, 3H). MS (m/z): 467.0 (M+H).

### Step 6. Diethyl 2-(2,2,2-trifluoro-1-(3-fluoro-4-(2-(1-methyl-1H-imidazol-4-yl)thieno[3,2-b]pyridin-7-yloxy)phenylamino)ethyl)malonate (6)

To a solution of 5 (470 mg, 1.01 mmol) and diethyl malonate (0.17 mL, 1.11 mmol) in anhydrous tetrahydrofuran (10 mL) under nitrogen was added sodium hydride (60% in oil, 89 mg, 2.22 mmol). The mixture was heated to reflux for 2 h, cooled, diluted with EtOAc and water and acidified to pH 3 using a 1N HCl solution. The organic layer was separated and the aqueous layer was extracted twice with EtOAc. The extracts were combined, dried over sodium sulfate and filtered. The filtrate was collected, solvents were removed under reduced pressure and the residue was purified by column chromatography on silica gel (eluent MeOH-dichloromethane, gradient 0:100 to 20:80) to afford 6 (490 mg, 84% yield). ¹H NMR (400 MHz, DMSO-d₆) δ (ppm): 8.42 (d, J=5.2 Hz, 1H), 7.85 (s, 1H), 7.72 (s, 1H), 7.67 (s, 1H), 7.24 (t, J=9.2 Hz, 1H), 6.98 (dd, J=13.6, 2.8 Hz, 1H), 6.72 (dd, J=9.2, 2.8 Hz, 1H), 6.62 (d, J=10.0 Hz, 1H), 6.46 (d, J=5.2 Hz, 1H), 5.05-4.95 (m, 1H), 4.23-4.07 (m, 4H), 3.91 (d, J=9.2 Hz, 1H), 3.72 (s, 3H), 1.18 (t, J=7.0 Hz, 3H), 1.11 (t, J=7.0 Hz, 3H). MS (m/z): 581.0 (M+H).

### Step 7. 4,4,4-Trifluoro-3-(3-fluoro-4-(2-(1-methyl-1H-imidazol-4-yl)thieno[3,2-b]pyridin-7-yloxy)phenylamino)butanoic acid (7)

A solution of 6 (490 mg, 0.84 mmol) and sodium hydroxide (338 mg, 8.44 mmol) in water (0.7 mL) and ethanol (3.4 mL) was stirred at room temperature for 48 h. The solvents were removed under reduced pressure and the residue was dissolved in water (20 mL). The aqueous solution was neutralized to pH 4 with a 3N HCl solution and the solid thus formed was collected by filtration, rinsed with water and dried. The dry solid was suspended in anhydrous toluene (20 mL), heated to reflux for 1 h under continuous stirring. The toluene was removed under reduced pressure and the residue was purified by column chromatography on silica gel (eluent MeOH-dichloromethane, gradient 10:90 to 50:50) to form a solid material which was triturated with a mixture of dichloromethane, ethyl acetate and hexane, to afford 7 (150 mg, 0.31 mmol, 37% yield). MS (m/z): 480.9 ((M+H).

### Step 8. 4,4,4-Trifluoro-3-(3-fluoro-4-(2-(1-methyl-1H-imidazol-4-yl)thieno[3,2-b]pyridin-7-yloxy)phenylamino)-N-phenylbutanamide (8)

To a stirred solution of 7 (150 mg, 0.31 mmol), aniline (43 μL, 0.47 mmol) and N,N-diisopropylethylamine (0.19 mL, 1.09 mmol) in dry DMF (4 mL) at room temperature was added the HATU reagent (356 mg, 0.94 mmol). The mixture was stirred at room temperature for 16 h. A saturated aqueous solution of sodium bicarbonate was added and the aqueous phase was extracted twice with EtOAc. The organic extract was dried over anhydrous sodium sulfate, filtered and the filtrate was concentrated under reduced pressure. The residue was purified by column chromatography on silica gel (eluent MeOH-dichloromethane, gradient 3:97 to 8:92) to afford the title compound 8 (111 mg, 0.20 mmol, 64% yield) as a white solid. Characterization of the compound is provided in the Table 1

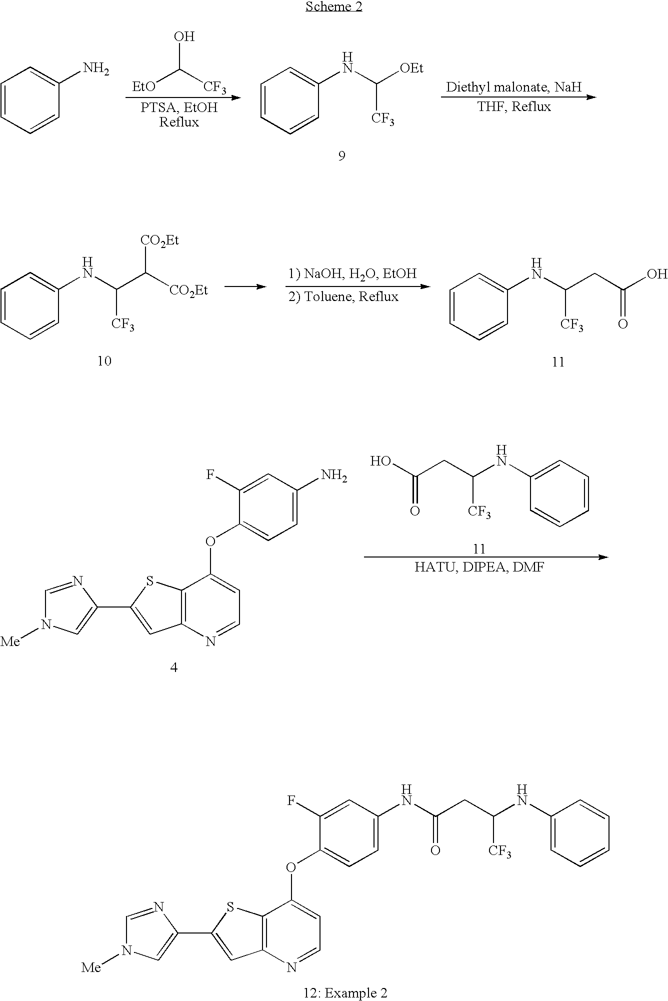

### Example 2

### 4,4,4-Trifluoro-N-(3-fluoro-4-(2-(1-methyl-1H-imidazol-4-yl)thieno[3,2-b]pyridin-7-yloxy)phenyl)-3-(phenylamino)butanamide (12)

### Step 1. N-(1-Ethoxy-2,2,2-trifluoroethyl)benzenamine (9)

A solution of aniline (2 mL, 21.9 mmol), trifluoroacetaldehyde ethyl hemiacetal (2.6 mL, 21.9 mmol) and p-toluenesulfonic acid monohydrate (220 mg, 1.16 mmol) in ethanol (25 mL) was heated to reflux for 3 h under continuous stirring. The reaction mixture was cooled, the solvent was removed under reduced pressure and the residue was dissolved in EtOAc. The solution was washed with saturated aqueous sodium bicarbonate, dried over anhydrous sodium sulfate, filtered and the filtrate was concentrated under reduced pressure to afford the title compound 9 (4.16 g, crude) as a yellow oil which was used directly for next step.

### Step 2. Diethyl 2-(2,2,2-trifluoro-1-(phenylamino)ethyl)malonate (10)

A solution of diethyl malonate (1.98 mL, 13.0 mmol) in anhydrous tetrahydrofuran (10 mL) was added drop-wise at 0° C. for 20 min into a suspension of sodium hydride (60% in oil, 0.52 g, 13.0 mmol) in dry tetrahydrofuran (30 mL), followed by the addition of 9 (2.6 g, 11.9 mmol). The mixture was heated to reflux for 16 h under vigorous stirring, cooled to room temperature and acidified to pH 3 using a 1N HCl solution. The acidic solution was extracted twice with EtOAc. The extracts were combined, dried over sodium sulfate, filtered and the solvents were removed under reduced pressure. The residue was purified by column chromatography on silica gel (eluent dichloromethane-hexane, 0:100 to 60:40) to afford 10 (2.16 g, 54% yield) as a colorless oil. ¹H NMR (400 MHz, CDCl₃) δ (ppm): 7.21-7.16 (m, 2H), 6.81-6.76 (m, 1H), 6.75-6.71 (m, 2H), 5.06 (d, J=10.4 Hz, 1H), 4.83-4.73 (m, 1H), 4.25 (q, J=7.2 Hz, 2H), 4.18-4.05 (m, 2H), 3.85 (d, J=4.4 Hz, 1H), 1.28 (t, J=7.2 Hz, 3H), 1.13 (t, J=7.2 Hz, 3H). MS (m/z): 334.1 (M+H).

### Step 3. 4,4,4-Trifluoro-3-(phenylamino)butanoic acid (11)

A solution of 10 (2.16 g, 6.48 mmol) and sodium hydroxide (2.60 g, 64.8 mmol) in water (5.2 mL)-ethanol (26 mL) mixture, was stirred at room temperature for 24 h. The solvents were removed under reduced pressure leaving a white solid which was triturated with ether, dried under high vacuum and dissolved in water (12 mL). The aqueous solution was neutralized to pH 4 with a 3N HCl solution, extracted twice with EtOAc, the combined organic extracts were dried over sodium sulfate, filtered and the solvent was removed under reduced pressure. The remaining solid was dissolved in dry toluene (20 mL), heated to reflux for 1 h under continuous stirring, and the solvent was removed under reduced pressure. The residue was purified by column chromatography on silica gel (eluent EtOAc-hexane, gradient 0:100 to 40:60) to afford 11 (204 mg, 0.87 mmol, 13% yield) as a beige solid. ¹H NMR (400 MHz, CDCl₃) δ (ppm): 7.24-7.18 (m, 2H), 6.82 (tt, J=7.2, 1.0 Hz, 1H), 6.75-6.71 (m, 2H), 4.55-4.45 (m, 1H), 2.89 (dd, J=16.0, 4.4 Hz, 1H), 2.67 (dd, J=16.0, 8.8 Hz, 1H). MS (m/z) 231.9 (M−1).

### Step 4. 4,4,4-Trifluoro-N-(3-fluoro-4-(2-(1-methyl-1H-imidazol-4-yl)thieno[3,2-b]pyridin-7-yloxy)phenyl)-3-(phenylamino)butanamide (12)

To a stirred solution of 4 (scheme 1) (100 mg, 0.29 mmol), 11 (103 mg, 0.44 mmol) and N,N-diisopropylethylamine (0.18 mL, 1.03 mmol) in dry DMF (3 mL) at 0° C. was added the HATU reagent (335 mg, 0.88 mmol). The mixture was stirred at room temperature for 16 h. Saturated aqueous sodium bicarbonate was added and the solution was extracted twice with EtOAc, dried over anhydrous sodium sulfate, filtered and evaporated under reduced pressure. The residue was purified by column chromatography on silica gel (eluent MeOH-dichloromethane, gradient 0:100 to 15:85). The resulting solid was triturated with in methanol, to afford the title compound 12 (81 mg, 50% yield) as a white solid. Characterization of the compound is provided in the Table 1

The following compounds, and the compounds described in the assay examples below, are prepared essentially according to the procedures outlined in the schemes, charts, examples and preparations set forth herein.

TABLE 1

| Ex. | Cpd | | |
|---|---|---|---|
| No | No. | Compound | Characterization |
| 1 | 8 | 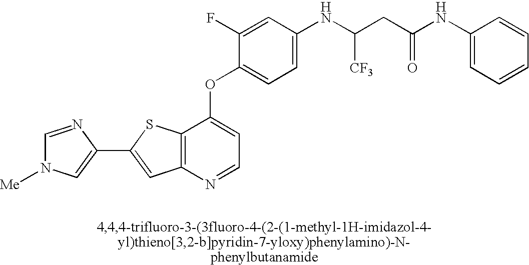 | ¹H NMR(400 MHz, DMSO-d₆) δ(ppm): 10.11(s, 1H), 8.39(d, J=5.6 Hz, 1H), 7.85(d, J=1.2 Hz, 1H), 7.72(d, J=0.8 Hz, 1H), 7.66 (s, 1H), 7.56(d, J=7.2 Hz, 2H), 7.30(t, J=8.0 Hz, 2H), 7.21(t, J=8.8 Hz, 1H), 7.05(t, J=7.2 Hz, 1H), 6.86(dd, J=13.6, 2.4 Hz, 1H), 6.67(dd, J=8.8, 2.0 Hz, 1H), 6.57(d, J=8.8 Hz, 1H), 6.44(d, J=5.6 Hz, 1H), 4.86-4.53(m, 1H), 3.72(s, 3H), 2.92(dd, J=15.6, 3.2 |
| # Hz, 1H), 2.76(dd, J=15.6, 9.6 Hz, 1H). MS(m/z):(M + H) 556.0 (100%). | | | |
| 2 | 12 | 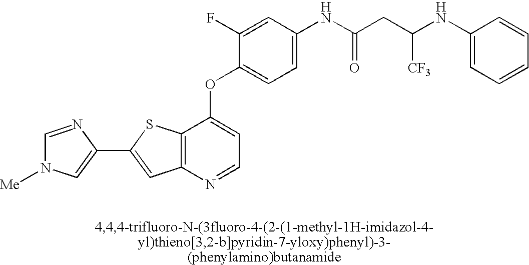 | ¹H NMR(400 MHz, DMSO-d₆) δ10.50(s, 1H), 8.41(d, J=5.6 Hz, 1H), (d, J=1.2 Hz, 1H), 7.81(dd, J=13 Hz, 1H), 7.72(s, 1H), 7.67(s, 1H), 7. 8.8 Hz, 1H), 7.37(dd, J=8.8, 1H), 7.10(dd, J=8.4, 7.2 Hz, 2H), J=8.0 Hz, 2H), 6.61(t, J=7.2 H J=5.6 Hz, 1H), 6.13(d, J=1H), 4.75-4.65(m, 1H), 3.72(s, 3H) (dd, J=15.6, 3.6 Hz, 1H), 2.78(15.9, 9.6 Hz, 1H). MS(m/z): 556.0( |
| 3 | 13 | 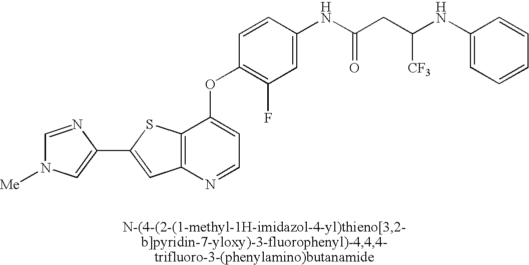 | ¹H NMR: DMSO) δ(ppm): 10.50(s, 1H), 8.41(d, J=5.5 Hz, 1H), 7.96(d, 1.2 Hz, 1H), 7.82-7.78(m, 2H), 7.66(s, 1H), 7.45(t, J=8.8 Hz, 1H), 7.37(dd, J=9.0/1.4 Hz, 1H), 7.11 (dd, J=8.4/7.4 Hz, 2H), 6.75(d, J=7.8 Hz, 2H) 6.61(t, J=7.3 Hz, 6.55(d, J=5.5 Hz, 1H), 6.12(d, J=9.2 Hz, 1H), 4.70(m, 1H), 4.06 (quad., J=7.2 Hz, 2H), 2.94(dd, J=15.6/3.9 Hz, 1H), 2.78(dd, J=15.9/9.3 Hz, 1H) 1.40(t, J=7.3 Hz, 3H). |
| # LRMS. 569.2(calc) 570.2(found, M- | | | |
| 4 | 14 | 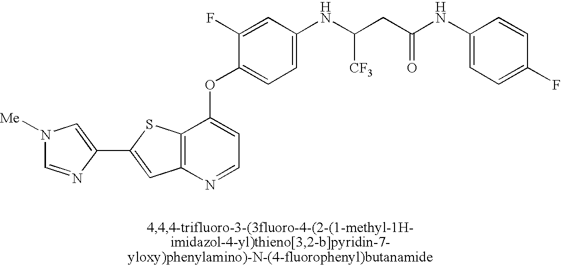 | ¹H NMR: (DMSO) δ(ppm): 10.18 (s, 1H), 8.39(d, J=5.2 Hz, 1H), 7.85(d, J=1.2 Hz, 1H), 7.72(d, J=1.2 Hz, 1H), 7.66(s, 1H), 7.61- 7.55(m, 2H), 7.21(t, J=8.8 Hz, 1H), 7.18-7.11(m, 2H), 6.85(dd, J=13.2, 2.4 Hz, 1H), 6.67(dd, J=8.8, 2.4 Hz, 1H), 6.57(d, J=9.2 Hz, 1H), 6.44(d, J=5.2 Hz, 1H), 4.85-4.73(m, 1H), 3.72(s, 3H), 2.91(dd, J=15.6, 4.0 Hz, 1H), 2.74(dd, J=15.6, 9.6 Hz, 1H). LRMS: 573.13(M, calc) 574.2 |
| # (M + H found) | | | |
| 5 | 15 | 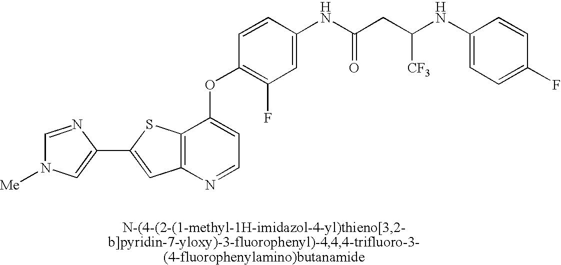 | ¹H NMR:(DMSO) δ(ppm): 10.47(s 1H), 8.41(d, J=5.5 Hz, 1H), 7.96(s, 1H), 7.82-7.78(m, 2H), 7.67(d, J=0.4 Hz, 1H), 7.45(t, J=8.9 Hz, 1H) J=8.8 Hz, 2H), 6.77-6.74(m, 2H), 6.55(d, J=5.3 Hz, 1H), 6.06(d, J=8.8 Hz, 1H), 4.64(m, 1H), 4.06(q, J=7.2 Hz, 2H), 2.93(dd, J=15.7/3.8 Hz, 1H), 2.76(dd, J=15.8/9.4 Hz, 1H), 1.40(t, J=7.2 Hz, 3H) LRMS: 587.1 (calc) 588.2(found, M |

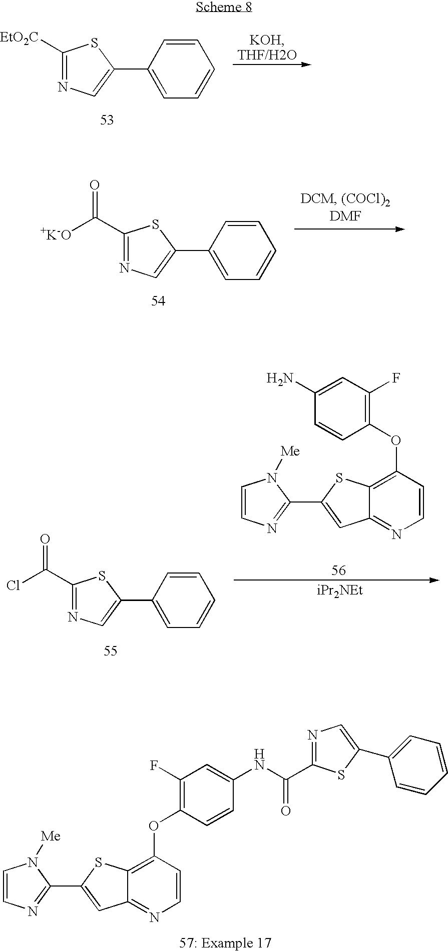

### Example 17

### N-(3-Fluoro-4-(2-(1-methyl-1H-imidazol-2-yl)thieno[3,2-b]pyridin-7-yloxy)phenyl)-5-phenylthiazole-2-carboxamide (57)

### Step 1. Potassium 5-phenylthiazole-2-carboxylate (54)

To a solution of 53 (Tanaka C., Nasu K., Yamamoto N., Shibata M. Chem. Pharm. Bull. 90, 11, 4195-4198) (156 mg, 0.669 mmol) in THF (2 mL) and water (2 mL) was added KOH (41.3 mg, 0.0.736 mmol) and the reaction mixture was stirred at RT for 3 hours. The mixture was concentrated and the resultant potassium salt was triturated with acetone to afford title compound 54 (155 mg, 95% yield), which was used directly in the next step with no additional purification. MS (m/z): 206.1 (M+H) (free acid).

### Step 2. 5-Phenylthiazole-2-carbonyl chloride (55)

To a suspension 54 (168 mg, 0.691 mmol) in DCM (4 mL) was added oxalyl chloride (87.6 mg, 0.691 mmol) and 1 drop of DMF. The reaction mixture was allowed to stir for 3 hours at RT. The solvent was removed and the residue—the title compound 55 (155 mg, 99% yield) was used directly in the next step.

### Step 3. N-(3-Fluoro-4-(2-(1-methyl-1H-imidazol-2-yl)thieno[3,2-b]pyridin-7-yloxy)phenyl)-5-phenylthiazole-2-carboxamide (57)

To a suspension of 3-fluoro-4-(2-(1-methyl-1H-imidazol-2-yl)thieno[3,2-b]pyridin-7-yloxy)aniline (56) [WO 2006/019264 A1] (150 mg, 0.441 mmol) in DCM (7 mL) was added acyl chloride 55 (197 mg, 0.881 mmol) and Hunig's base (171 mg, 1.322 mmol) and the reaction mixture was stirred at RT for 24 hours. The solid precipitate was collected by filtration, then washed with MeOH, acetone and water to give title compound 57 (100 mg, 43% yield) as a beige solid. ¹H NMR (400 MHz, DMSO-d₆) o (Ppm) 11.23 (s, 1H), 8.55 (s, 1H), 8.52 (m, 1H), 8.08 (d, J=13.1 Hz, 1H), 7.82 (m, 4H), 7.5 (m, 5H), 7.04 (s, 1H), 6.71 (d, J=5.3 Hz, 1H), 3.99 (s, 3H). MS (m/z): 528.2 (M+H)

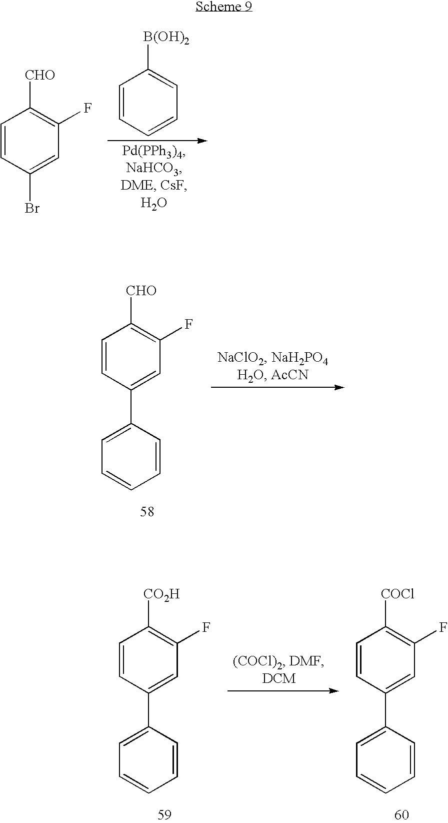

### Step 1. 3-Fluorobiphenyl-4-carbaldehyde (58)

To a solution of 4-bromo-2-fluorobenzaldehyde (1.0 g, 4.93 mmol) in DME (50 mL) was added phenylboronic acid (0.901 g, 7.39 mmol), cesium fluoride (2.245 g, 14.78 mmol), NaHCO₃ (1.241 g, 14.78 mmol) in water (17 mL) and Pd(PPh₃)₄ (0.569 g, 0.493 mmol). The reaction mixture was heated to reflux for 3 hours. The reaction was cooled to RT, and concentrated then partitioned between water and EtOAc. The organic phase was collected, dried over Na₂SO₄, filtered and concentrated to afford a brown oil which was purified by column chromatography (eluent 10% EtOAc/Hexane) to afford 58 (900 mg, 91% yield). ¹H NMR (400 MHz, DMSO-d₆) o (ppm) 10.39 (s, 1H) 7.93 (m, 1H), 7.62 (m, 2H), 7.45 (m, 5H).

Step 2. 3-Fluorobiphenyl-4-carboxylic acid (59).

To a solution of 58 (900 mg, 4.50 mmol) in MeCN (40 mL) was added sodium dihydrogenphosphate (2.697 g, 22.48 mmol) and water (20 mL). The mixture was stirred for 5 min before the addition of sodium chlorite (4.066 g, 22.48 mmol) in water (20 mL). It was then stirred for an additional 30 min and the resultant solid was collected by filtration to afford title compound 59 (900 mg, 93% yield), which was used without additional purification. MS (m/z): 217.1 (M+H)

### Step 3. 3-Fluorobiphenyl-4-carbonyl chloride (60)

To a suspension of 59 (200 mg, 0.925 mmol) in DCM (5 mL) was added DMF (1 drop) and oxalyl chloride (235 mg, 1.850 mmol) and the reaction mixture was stirred for 30 min. The mixture was concentrated to dryness to afford title compound 60 (210 mg, 97% yield), which was used directly in the synthesis of compound 61 (example 18), with no additional purification.

### Example 18

### 3-Fluoro-N-(3-fluoro-4-(2-(1-methyl-1H-imidazol-2-yl)thieno[3,2-b]pyridin-7-yloxy)phenyl)biphenyl-4-carboxamide (61)

Following the procedure described above for the synthesis of compound 57 (scheme 8, example 17) but replacing the acyl chloride 55 with the acyl chloride 60 (scheme 9), title compound 61 was obtained in 22% yield. Characterization of 61 is provided in the Table 2.

TABLE 2

| Cmpd. # | Ex. # | Structure | Characterization |
|---|---|---|---|
| 61 | 18 | 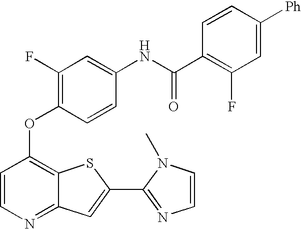 | ¹H NMR(400 MHz, DMSO-d₆) δ(ppm) 10.21(s, 1H), 8.53(d, J=5.5 Hz, 1H), 7.97(m, 1H), 7.89- 7.41(m, 12H), 7.05(s, 1H), 6.73(d, J=5.5 Hz, 1H), 3.98(s, 3H). MS(m/z): 539.2(M + H). |
| 62 | 19 | 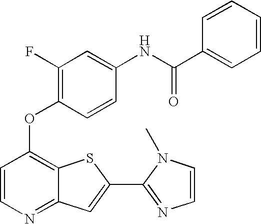 | ¹H NMR(400 MHz, DMSO-d₆) δ(ppm) 10.61(s, 1H), 8.54(d, J=5.5 Hz, 1H), 8.05(m, 1H), 7.98(m, 2H), 7.90(s, 1H), 7.56(m, 6H), 7.05(s, 1H), 6.78 (m, 1H), 4.0(s, 3H). MS(m/z): 445.5(M + H). |
| 63 | 20 | 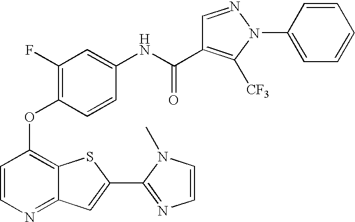 | ¹H NMR(400 MHz, DMSO-d₆) δ(ppm) 10.92(s, 8.54(d, J=5.5 Hz, 1H), 8.36(s, 1H), 7.96(m, 1H), 7.91(s, 1H), 7.56(m, 7H), 7.42(s, 1H), 7.05(s, 1H), 6.72(m, 1H), 4.0(s, 3H)(mono-formate salt). MS(m/z): 579.49(M + H). |
| 67 | 21 | 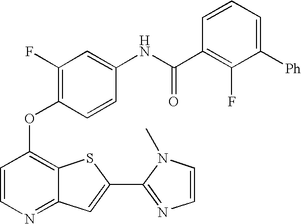 | ¹H NMR(400 MHz, DMSO-d₆) δ(ppm) 10.89(s, 1H), 8.52(d, J=5.5 Hz, 1H), 7.98(m, 1H), 7.90(s, 1H), 7.72-7.41(m, 1 1H), 7.0(s, 1H), 6.71(d, J=5.5 Hz, 1H), 3.99(s, 3H). MS(m/z): 539.2(M + H). |
| 68 | 22 | 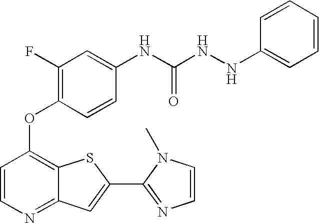 | ¹H NMR(400 MHz, DMSO-d₆) δ(ppm) 9.71(s, 1H) 8.48(d, J=5.5 Hz,1H), 8.41(s, 1H), 8.13(s, 1H), 8.04(s, 1H), 7.85(m, 4H), 7.46(m, 3H), 7.38(t, J=9.2 Hz, 1H), 7.22(t, J=8.2 Hz, 1H), 6.78(m, 3H), 6.60(d, J=5.5 Hz, 1H), 3.62(s, 2 H), 3.33(s, 3H), 3.24(s, 3H), 3.15(m, 1H)(mono-forinate salt). MS(m/z): 572.56(M + H). |

### Example 19

### N-(3-Fluoro-4-(2-(1-methyl-1H-imidazol-2-yl)thieno[3,2-b]pyridin-7-yloxy)phenyl)benzamide (62)

Following the procedure described above for the synthesis of compound 57 (scheme 8, example 17) but replacing the acyl chloride 55 with benzoyl chloride title compound 62 was obtained in 38% yield. Characterization of 62 is provided in the Table 2.

### Example 20

### N-(3-Fluoro-4-(2-(1-methyl-1H-imidazol-2-yl)thieno[3,2-b]pyridin-7-yloxy)phenyl)-1-phenyl-5-(trifluoromethyl)-1H-pyrazole-4-carboxamide (63)

Following the procedure described above for the synthesis of compound 57 (scheme 8, example 17) but replacing the acyl chloride 55 with 1-phenyl-5-(trifluoromethyl)-1H-pyrazole-4-carboxylic acid and EDC as a coupling agent, title compound 63 was obtained in 15% yield. Characterization of 63 is provided in the Table 2.

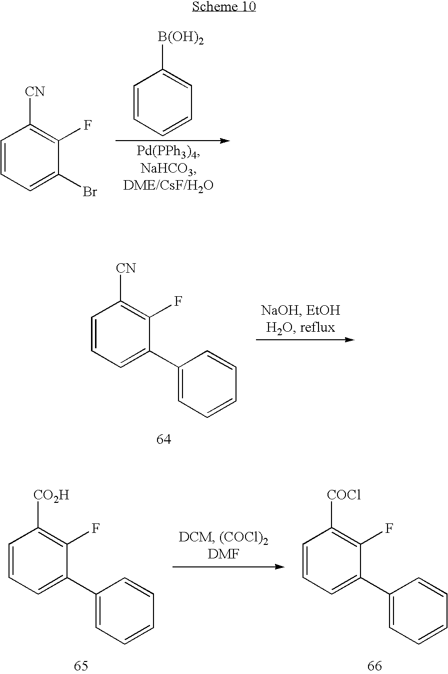

### Step 1. 2-Fluorobiphenyl-3-carbonitrile (64)

To a solution of 3-bromo-2-fluorobenzonitrile (1.0 g, 4.93 mmol) in DME (50 mL) was added phenylboronic acid (0.901 g, 7.39 mmol), cesium fluoride (2.245 g, 14.78 mmol), NaHCO₃ (1.241 g, 14.78 mmol) in water (17 mL) and Pd(PPh₃)₄ (0.569 g, 0.493 mmol). The reaction mixture was heated to reflux for 3 hours. The reaction was cooled to RT, and concentrated then partitioned between water and EtOAc. The organic phase was collected, dried over Na₂SO₄, filtered and concentrated to afford a brown oil which was purified by flash column chromatography (eluent 10% EtOAc/Hexane), to afford 64 (1.5 g, 65% yield) as a white solid. ¹H NMR (400 MHz, DMSO-d₆) δ (ppm) 7.71-6.79 (m, 8H).

### Step 2. 2-Fluorobiphenyl-3-carboxylic acid (65)

To a solution of 64 (1.4 g, 7.1 mmol) in EtOH (20 mL) and water (10 mL) was added NaOH (0.568 g, 14.2 mmol) and the reaction was heated to reflux for 3 hours. It was then cooled to RT, concentrated and the residue was partitioned between Et₂O and water. Organic layer was discarded. The aqueous phase was acidified to pH 1 and then extracted with EtOAc. The organic extract was dried over Na₂SO₄, filtered and evaporated. The residue was triturated with hexane to afford title compound 65 (420 mg, 27% yield), which was used without additional purification. MS (m/z): 217.1 (M+H).

### Step 3. 2-Fluorobiphenyl-3-carbonyl chloride (66)

To a suspension of 65 (260 mg, 1.203 mmol) in dry DCM (10 mL) was added DMF (1 drop) and oxalyl chloride (305 mg, 2.405 mmol) and the reaction mixture was stirred at RT for 1 hour. The acyl chloride 66 (282 mg, 100% yield) was concentrated to dryness and used immediately in the synthesis of compound 67 (example 21).

### Example 21

### 2-Fluoro-N-(3-fluoro-4-(2-(1-methyl-1H-imidazol-2-yl)thieno[3,2-b]pyridin-7-yloxy)phenyl)biphenyl-3-carboxamide (67)

Following the procedure described above for the synthesis of compound 57 (scheme 8, example 17) but replacing the acyl chloride 55 with the acyl chloride 66 (scheme 10), title compound 67 was obtained in 13% yield. Characterization of 67 is provided in the Table 2.

### Example 22

### N-(3-Fluoro-4-(2-(4-(((2-methoxyethyl)(methyl)amino)methyl)phenyl)thieno[3,2-b]pyridin-7-yloxy)phenyl)-2-phenylhydrazinecarboxamide (68)

To a solution of 4-nitrophenylchloroformate (182 mg, 0.905 mmol) in THF (7 mL) at 0° C. was added Hunig's base (117 mg, 0.905 mmol) and the mixture was stirred for 5 min. It was then cooled to −30° C. and a solution of the amine 56 [WO 2006/019264 A1] (198 mg, 0.453 mmol) in THF (1 mL) was added. The reaction mixture was warmed slowly to RT whereupon phenylhydrazine (245 mg, 2.263 mmol) was added. The mixture was stirred at RT for 24 hours, diluted with EtOAc and washed with water and saturated NaHCO₃ solution, dried over Na₂SO₄, filtered and concentrated to afford a black oil which was purified by flash column chromatography (eluent 10% MeOH in EtOAc) and by Gilson Reverse Phase HPLC (Aquasil C₁₈, eluent a linear gradient of 30% MeOH in water to 95% MeOH in water, 60 min) to give 68 (55 mg, 21% yield) as a yellow solid. Characterization of 68 is provided in the Table 2.

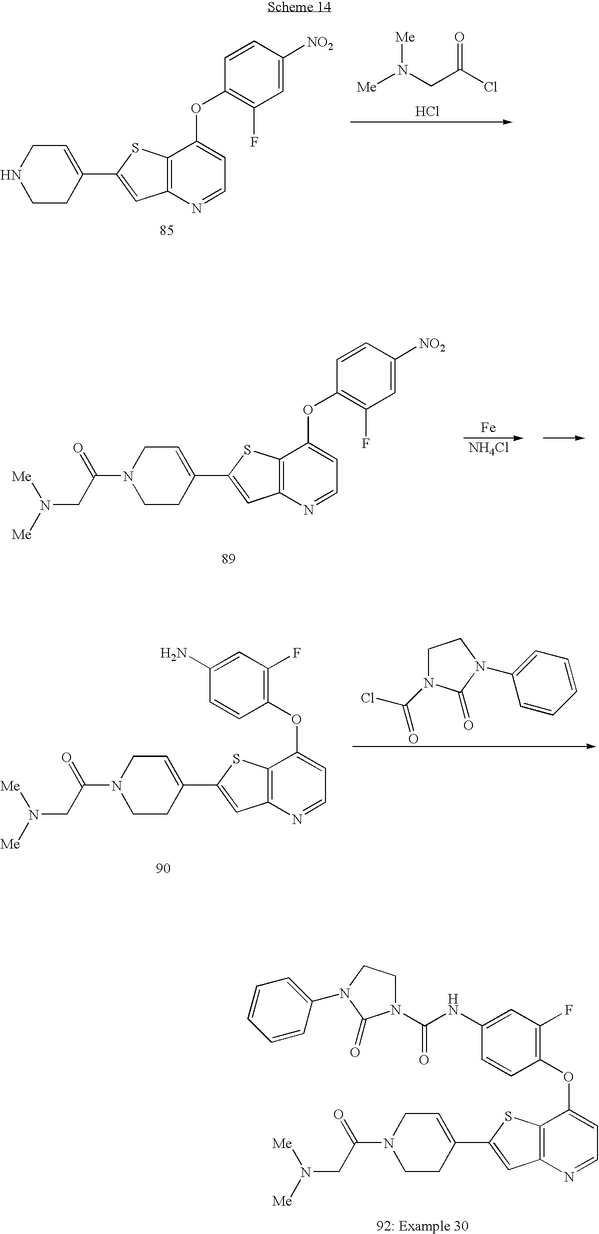

### Example 30

### N-(4-(2-(1-(2-(Dimethylamino)acetyl)-1,2,3,6-tetrahydropyridin-4-yl)thieno[3,2-b]pyridin-7-yloxy)-3-fluorophenyl)-2-oxo-3-phenylimidazolidine-1-carboxamide (92)

### Step 1. 2-(Dimethylamino)-1-(4-(7-(2-fluoro-4-nitrophenoxy)thieno[3,2-b]pyridin-2-yl)-5,6-dihydropyridin-1(2H)-yl)ethanone (89)

DIPEA (147.8 mL, 0.85 mmol) was added to a suspension of 7-(2-fluoro-4-nitrophenoxy)-2-(1,2,3,6-tetrahydropyridin-4-yl)thieno[3,2-b]pyridine (85), (US 2007/0004675 A1) (150 mg, 0.4 mmol) and 2-(dimethylamino)acetyl chloride hydrochloride (127.7 mg, 0.81 mmol) in DCM and the mixture was stirred for 1 h at room temperature. More DIPEA (29.5 mL, 0.17 mmol) and 2-(dimethylamino)acetyl chloride hydrochloride (25.5 mg, 0.16 mmol) were added and the reaction mixture was stirred for an additional hour. It was then transferred onto a silica gel chromatography column and eluted with 5% MeOH in DCM to afford the title compound 89 (100 mg, 54% yield) as creamy foam. MS (m/z): 475.2 (M+1).

### Step 2. 1-(4-(7-(4-Amino-2-fluorophenoxy)thieno[3,2-b]pyridin-2-yl)-5,6-dihydropyridin-1(2H)-yl)-2-(dimethylamino)ethanone (90)

Title compound 90 was obtained starting from the nitro compound 89 and following the same procedure as described above for the synthesis of amine 4 (scheme 1, step 4). MS (m/z): 427.4 (M+1).

### Step 3. N-(4-(2-(1-(2-(Dimethylamino)acetyl)-1,2,3,6-tetrahydropyridin-4-yl)thieno[3,2-b]pyridin-7-yloxy)-3-fluorophenyl)-2-oxo-3-phenylimidazolidine-1-carboxamide (92)

Title compound 92 was obtained starting from the amino compound 90 and following the same procedure as described above for the synthesis of compound 57 (example 18). ¹H NMR (400 MHz, DMSO-d₆) δ (ppm): 10.61 (s, 0.8H), 10.60 (s, 0.4H), 8.46 (d, J=5.3 Hz, 1H), 8.22 (s, 0.4H), 7.84 (d, J=12.1 Hz, 1H), 7.65-7.63 (m, 2H), 7.54 (m, 1H), 7.45-7.42 (m, 4H), 7.19 (t, J=7.3 Hz, 1H), 6.62 (d, J=5.3 Hz, 1H), 6.43 (m, 1H), 4.35 (m, 0.8H), 4.17 (m, 1.2H), 3.97 (s, 4H), 3.77 (m, 1.2H), 3.72 (m, 0.8H), 3.17 (s, 1.2H), 3.13 (s, 0.8H), 2.7 (m, 1.2H), 2.6 (m, 0.8H), 2.22 (s, 3.6H), 2.21 (s, 2.4H). MS (m/z): 615.3 (M+1).

Compounds 118-121 (examples 44-47) were prepared starting from 3-fluoro-4-(2-(1-methyl-1H-imidazol-4-yl)thieno[3,2-b]pyridin-7-yloxy)benzenamine (4) (scheme 1) according to the synthetic procedures similar to ones used to prepare compound 63 (example 20, table 2). Compound 122 (example 48) was synthesized from its bis-Boc-protected intermediate. Characterization of compounds 118-122 (examples 44-48) is provided in the table 3.

TABLE 3

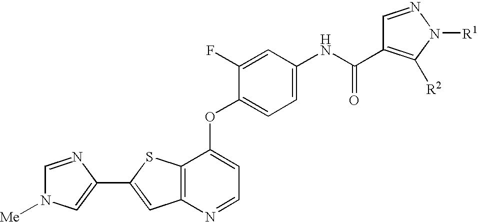

118-122: Examples 44-48

| Cpd | Ex. | R¹ | R² | Name | Characterization |
|---|---|---|---|---|---|
| 118 | 44 | Ph | CF₃ | N-(3-fluoro-4-(2-(1-methyl-1H- | ¹H NMR(400 MHz, DMSO-d₆) δ(ppm): |
| | | | | imidazol-4-yl)thieno[3,2-b]pyridin-7- | 10.91(s, 1H), 8.45(d, J=5.5 Hz, 1H), |
| | | | | yloxy)phenyl)-1-phenyl-5- | 8.37(s, 1H), 7.95(dd, J=12.8, 2.2 Hz, |
| | | | | (trifluoromethyl)-1H-pyrazole-4- | 1H), 7.87(d, J 1.2 Hz, 1H), 7.73(d, J= |
| | | | | carboxamide | 1.0 Hz, 1H), 7.70(s, 1H), 7.66-7.50(m, |
| | | | | | 7H), 6.62(dd, J=5.4, 0.7 Hz, 1H), 3.73 |
| | | | | | (s, 3H). MS(m/z): 579.2(M+H). |
| 119 | 45 | Ph | Me | N-(3-fluoro-4-(2-(1-methyl-1H- | ¹H NMR(400 MHz, DMSO-d₆) δ(ppm): |
| | | | | imidazol-4-yl)thieno[3,2-b]pyridin-7- | 10.20(s, 1H), 8.45(d, J=5.3 Hz, 1H), |
| | | | | yloxy)phenyl)-5-methyl-1-phenyl-1H- | 8.34(s, 1H), 8.00(dd, J=13.3, 2.3 Hz, |
| | | | | pyrazole-4-carboxamide | 1H), 7.87(d, J 1.2 Hz, 1H), 7.72(d, J= |
| | | | | | 0.8 Hz, 1H), 7.69(s, 1H), 7.65-7.46(m, |
| | | | | | 7H), 6.61(d, J=5.3 Hz, 1H), 3.73(s, |
| | | | | | 3H), 2.57(s, 3H). MS(m/z): 525.0 |
| | | | | | (M + H). |
| 120 | 46 | Ph | H | N-(3-fluoro-4-(2-(1-methyl-1H- | ¹H NMR(400 MHz, DMSO-d₆) δ(ppm): |
| | | | | imidazol-4-yl)thieno[3,2-b]pyridin-7- | 10.33(s, 1H), 9.15(s, 1H), 8.45(d, J= |
| | | | | yloxy)phenyl)-1-phenyl-1H-pyrazole- | 5.3 Hz, 1H), 8.36(s, 1H), 8.01(dd, J= |
| | | | | 4-carboxamide | 13.2, 2.2 Hz, 1H), 7.92(d, J=8.0 Hz, |
| | | | | | 2H), 7.87(s, 1H), 7.72(s, 1H), 7.69(s, |
| | | | | | 1H), 7.65-7.48(m, 4H), 7.40(t, J=7.3 |
| | | | | | Hz, 1H), 6.62(d, J=5.3 Hz, 1H), 3.73(s, |
| | | | | | 3H). MS(m/z): 511.1(M + H). |
| 121 | 47 | Me | CF₃ | N-(3-fluoro-4-(2-(1-methyl-1H- | ¹H NMR(400 MHz, DMSO-d₆) δ(ppm): |
| | | | | imidazol-4-yl)thieno[3,2-b]pyridin-7- | 10.49(s, 1H), 8.56(s, 1H), 8.44(d, J= |
| | | | | yloxy)phenyl)-1-methyl-5- | 5.5 Hz, 1H), 7.93(dd, J=13.0, 2.2 Hz, |
| | | | | (trifluoromethyl)-1H-pyrazole-4- | 1H), 7.87(d, J=1.4 Hz, 1H), 7.72(d, J= |
| | | | | carboxamide | 1.2 Hz, 1H), 7.69(s, 1H), 7.54(dd, J= |
| | | | | | 9.1, 2.2 Hz, 1H), 7.50(t, J 8.7 Hz, 1H), |
| | | | | | 6.60(dd, J=5.4, 0.7 Hz, 1H), 4.01(s, |
| | | | | | 3H), 3.73(s, 3H). MS(m/z): 517.1 |
| | | | | | (M + H). |
| 122 | 48 | Ph | NH₂ | 5-amino-N-(3-fluoro-4-(2-(1-methyl- | ¹H NMR(400 MHz, DMSO-d₆) δ(ppm): |
| | | | | 1H-imidazol-4-yl)thieno[3,2- | 9.90(s, 1H), 8.45(d, J=5.5 Hz, 1H), |
| | | | | b]pyridin-7-yloxy)phenyl)-1-phenyl- | 8.20(s, 1H), 7.98(dd, J=13.4, 2.4 Hz, |
| | | | | 1H-pyrazole-4-carboxamide | 1H), 7.87(d, J 1.2 Hz, 1H), 7.72(d, J= |
| | | | | | 1.0 Hz, 1H), 7.69(s, 1H), 7.62-7.52(m, |
| | | | | | 5H), 7.48(t, J 9.0 Hz, 1H), 7.42(tt, J= |
| | | | | | 7.0, 1.3 Hz, 1H), 6.61(dd, J=5.5, 0.6 |
| | | | | | Hz, 1H), 3.73(s, 3H). MS(m/z): 526.0 |
| | | | | | (M + H). |

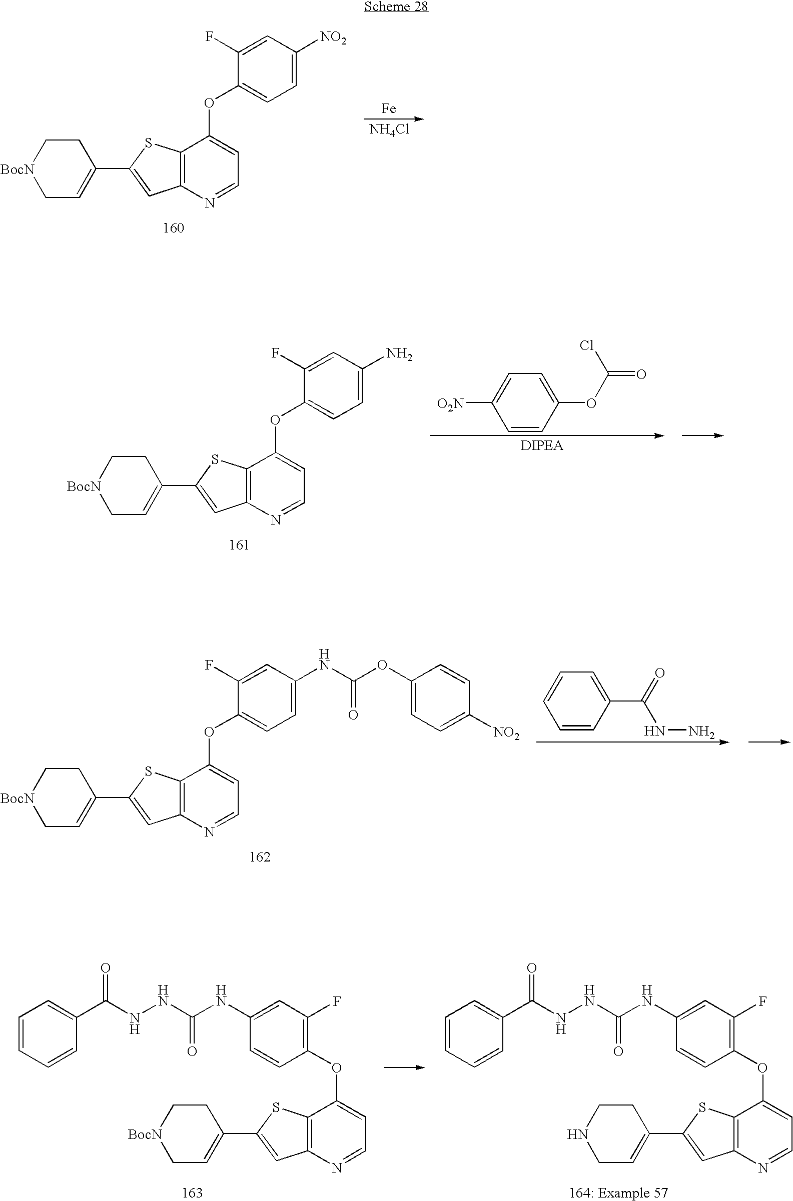

### Example 57

### 2-Benzoyl-N-(3-fluoro-4-(2-(1,2,3,6-tetrahydropyridin-4-yl)thieno[3,2-b]pyridin-7-yloxy)phenyl)hydrazinecarboxamide (164)

### Step 1. tert-Butyl 4-(7-(4-amino-2-fluorophenoxy)thieno[3,2-b]pyridin-2-yl)-5,6-dihydropyridine-1(2H)-carboxylate (161)

Iron powder (0.255 g, 4.56 mmol) was added to a hot mixture of tert-butyl 4-(7-(2-fluoro-4-nitrophenoxy)thieno[3,2-b]pyridin-2-yl)-5,6-dihydropyridine-1(2H)-carboxylate (160) [US 2007/0004675 A1] (0.2689 g, 0.570 mmol) and ammonium chloride (0.026 g, 0.485 mmol) in ethanol (5.43 ml) and water (2.72 ml) and was heated to reflux under vigorous stirring for 40 min. The mixture was filtered through a Celite® pad, the filtrate was collected and concentrated under reduced pressure. The residue was dissolved in DCM, and washed with water; the organic phase was dried over anhydrous Na₂SO₄ and concentrated under reduced pressure affording title compound 161 (0.2512 g, 0.569 mmol, 100% yield) as white foam. MS (m/z): 442.2 (M+H).

### Step 2. tert-Butyl 4-(7-(2-fluoro-4-((4-nitrophenoxy)carbonylamino)phenoxy)thieno[3,2-b]pyridin-2-yl)-5,6-dihydropyridine-1(2H)-carboxylate (162)

4-Nitrophenyl chloroformate (0.126 g, 0.626 mmol) was added to a mixture of the amine 161 (0.2512 g, 0.569 mmol) and DIPEA (0.119 ml, 0.683 mmol) in DCM (5.69 ml). The reaction mixture was stirred at room temperature overnight to form title compound 162 (0.569 mmol). The reaction mixture was used in the next step without isolation of 162. MS (m/z): 607.2 (M+H).

### Step 3. tert-Butyl 4-(7-(4-(2-benzoylhydrazinecarboxamido)-2-fluorophenoxy)thieno[3,2-b]pyridin-2-yl)-5,6-dihydropyridine-1(2H)-carboxylate (163)

Benzoic hydrazide (0.116 g, 0.854 mmol) was added to the solution of 162 (˜0.569 mmol) (see previous step) and the mixture was heated to reflux for 1 h. It was then concentrated under reduced pressure, the residue was dissolved in methanol and purified by preparative HPLC (column: Luna C18 (2), 5 cm ID; gradient: 60% MeOH to 95% MeOH in water, 60 min) affording title compound 163 (0.0343 g, 0.057 mmol, 10% yield) as white solid. MS (m/z): 604.3 (M+H).

### Step 4. 2-Benzoyl-N-(3-fluoro-4-(2-(1,2,3,6-tetrahydropyridin-4-yl)thieno[3,2-b]pyridin-7-yloxy)phenyl)hydrazinecarboxamide (164)

A solution of 163 (0.0195 g, 0.032 mmol) in TFA (1 mL) was stirred for 1 h at room temperature. It was then concentrated under reduced pressure. The residue was co-distilled with DCM and MeCN; dissolved in water and lyophilized affording title compound 164 (0.023 g, 99% yield). ¹H-NMR (400 MHz, CD₃OD): 8.60 (br, 1H), 7.93 (dd, J=7.0, 1.5 Hz, 2H), 7.73 (dd, J=13.1, 2.3 Hz, 1H), 7.67-7.56 (m, 3H), 7.56-7.49 (m, 2H), 7.39-7.31 (m, 2H), 6.91 (d, J=6.0 Hz, 1H), 6.58 (m, 1H), 3.96 (m, 2H), 3.54 (t, J=6.1 Hz, 2H), 2.97 (m, 2H) (presumably di-trifluoroacetate salt). MS (m/z): 505.2 (M+H).

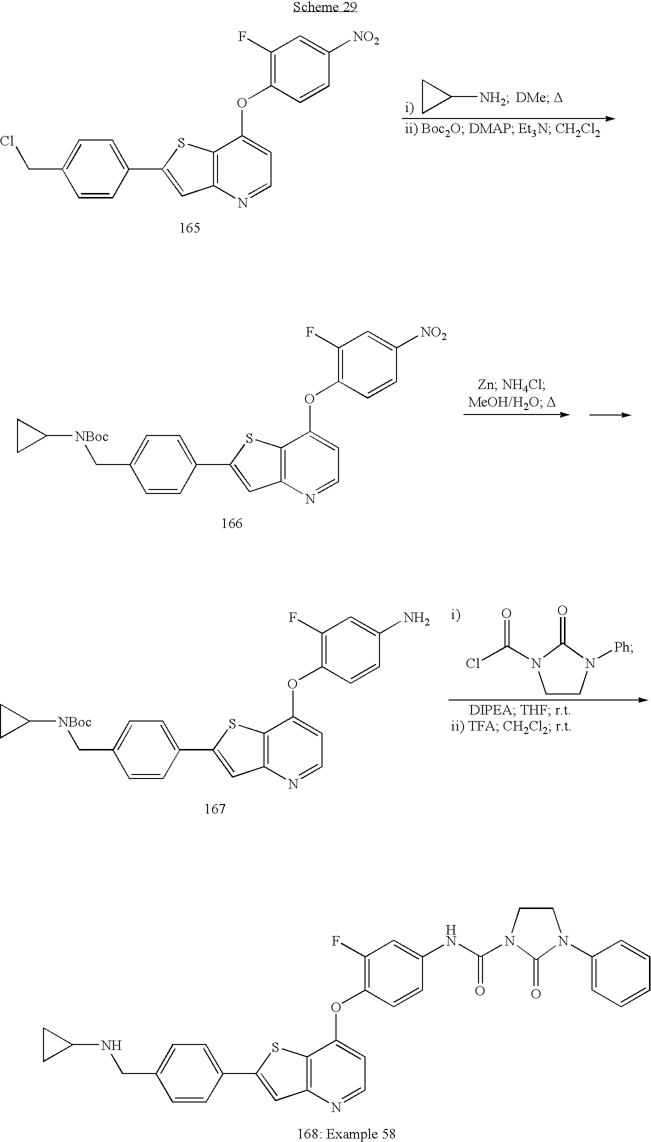

### Example 58

### N-(4-(2-(4-((cyclopropylamino)methyl)phenyl)thieno[3,2-b]pyridin-7-yloxy)-3-fluorophenyl)-2-oxo-3-phenylimidazolidine-1-carboxamide (168)

### Steps 1 and 2: tert-Butyl cyclopropyl(4-(7-(2-fluoro-4-nitrophenoxy)thieno[3,2-b]pyridin-2-yl)benzyl)carbamate (166)

To a suspension of 2-(4-(chloromethyl)phenyl)-7-(2-fluoro-4-nitrophenoxy)thieno[3,2-b]pyridine (165) [US 2006/0287343 A1] (1.50 g, 3.6 mmol) in DME (50 mL) was added cyclpropylamine (4 mL) in a pressure flask. The flask was sealed and the reaction mixture heated to 90° C. for 2 hrs, then cooled. The solvent was removed under reduced pressure; the residue was dissolved in EtOAc and washed with water and brine. The organic phase was collected, dried over sodium sulfate, filtered and concentrated. To the resulting crude product in dichloromethane (50 mL) at room temperature was added triethylamine (1.0 mL, 7.1 mmol), DMAP (0.055 g, 0.63 mmol) and Boc₂O (1.15 g, 5.3 mmol). The reaction mixture was stirred at room temperature overnight, then the mixture was washed sequentially with water, and brine; dried over anhydrous sodium sulfate, filtered, and concentrated. The residue was purified by column chromatography (eluent 75% ethyl acetate/hexanes) to afford title compound 166 (0.70 g, 36% yield) as a yellow oil. MS (m/z): 536.2. (M+H):

### Step 3: tert-Butyl 4-(7-(4-amino-2-fluorophenoxy)thieno[3,2-b]pyridin-2-yl)benzyl(cyclopropyl)carbamate (167)

To the nitro compound 166 (0.70 g, 1.3 mmol) MeOH (50 mL) was added Zn dust (1.0 g, 15 mmol) and ammonium chloride (0.125 g, 2.3 mmol) in water (5 mL). The resulting mixture was heated to reflux for 2 h, then filtered through celite and concentrated. Silica gel chromatography (eluent 70% ethyl acetate/hexanes) provided 167 (0.61 g, 92% yield). ¹H NMR (400 MHz, DMSO-d₆) δ (ppm): 8.46 (d, J=5.5, 1H); 7.99 (s, 1H); 7.85 (d, J=8.2, 2H); 7.33 (d, J=8.4, 2H); 7.11 (t, J=9.0, 1H); 6.55-6.51 (m, 2H); 6.46-6.43 (m, 1H); 5.54 (s, 2H); 4.40 (m, 2H); 2.43 (m, 1H); 1.41 (s, 9H); 0.67-0.59 (m, 4H). MS (m/z): 506.3 (M+H).

### Steps 4 and 5: N-(4-(2-(4-((Cyclopropylamino)methyl)phenyl)thieno[3,2-b]pyridin-7-yloxy)-3-fluorophenyl)-2-oxo-3-phenylimidazolidine-1-carboxamide (168)

To a suspension of aniline 167 (0.10 g, 0.20 mmol) and DIPEA (0.25 mL, 0.19 g, 1.4 mmol) in dry THF (25 mL) was added a suspension of freshly prepared 2-oxo-3-phenylimidazolidine-1-carbonyl chloride [US 2007/0004675 A1] (0.07M in THF, 4.0 mL, 0.28 mmol) and the resulting mixture was stirred at r.t. for 3 h. It was then concentrated and the residue was purified by silica gel chromatography (eluent 90% ethyl acetate/hexanes). The product was dissolved in dichloromethane (20 mL) and trifluoroacetic acid (2 mL) and stirred for 18 h at r.t. The mixture was concentrated and purified by reverse phase HPLC (Aquasil C-18 column, 35-85% MeOH/H₂O+HCO₂H, 30 min. linear gradient elution) and lyophilization, to afford title compound 168 (0.062 g, 52% yield). Characterization of 168 is provided in the table 4.

TABLE 4

| Cmpd. | Ex. | | |
|---|---|---|---|
| # | # | Structure | Characterization |
| 168 | 58 | 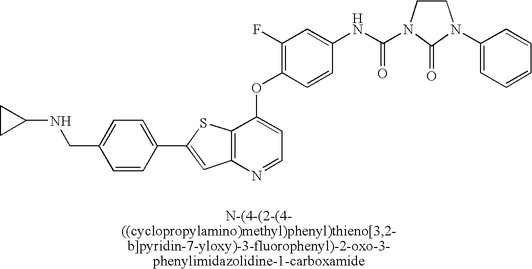 | ¹H NMR(400 MHz, DMSO-d₆) δ(ppm): 10.59(s, 1H), 8.51(d, J=5.5 Hz, 1H), 8.03(s, 1H), 7.86(dd, J=13.3, 2.5 Hz, 1H), 7.85-7.82(m, 2H), 7.65-7.62(m, 2H), 7.50-7.41(m, 6H), 7.20-7.16(m, 1H), 6.64(dd, J=5.3, 1.0 Hz, 1H), 3.98-3.94(m, 4H), 3.77 (s, 2H), 2.04(septet, J=3.5 Hz, 1H), 0.37-0.33(m, 2H), 0.28-0.25(m, 2H). MS(m/z): 594.3(M + H). |
| 169 | 59 | 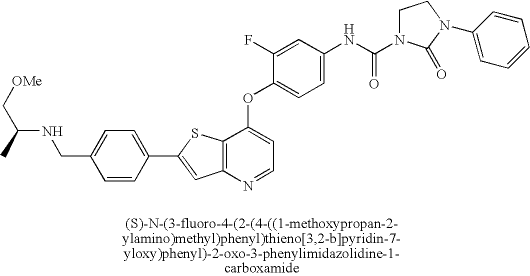 | ¹H NMR(400 MHz, DMSO-d₆) δ(ppm) :10.58(s, 1H), 8.51(d, J=5.5 Hz, 1H), 8.02(s, 1H), 7.89-7.79(m, 3H), 7.63(d, J=8.2 Hz, 2H), 7.54- 6.64(d, J=5.3 Hz, 1H), 4.02-3.90(m, 4H), AB system(□_{A} =3.82, □_{B} =3.74, J=14.0 Hz, 2H), 3.30-3.15(m, 5H), 2.82-2.72(m, 1H), 0.98(d, J=6.5 Hz, 3H), one NH is missing. MS (m/z): 626.3(M + H). |
| 170 | 60 | 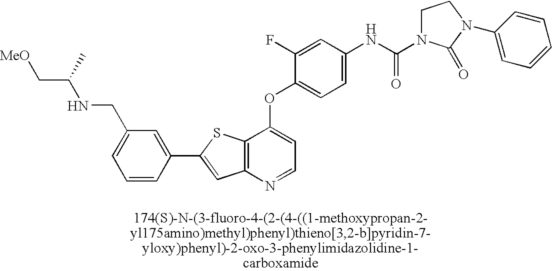 | ¹H NMR(400 MHz, DMSO-d₆) δ(ppm): 10.59(s, 1H), 8.52(d, J=5.5 Hz, 1H), 8.04(s, 1H), 7.90-7.83(m, 2H), 7.76(d, J 7.2 Hz, 1H), 7.64(d, J=7.8 Hz, 2H), 7.55-7.38(m, 6H), 7.18(t, J=7.4 Hz, 1H), 6.65(d, J=5.3 Hz, 1H), 4.05-3.90(m, 4H), 3.87 (d, J=13.9 Hz, 1H), 3.79(d, J=13.9 Hz, 1H), 3.35-3.15(m, SH), 2.81(hex, J=6.1 Hz, 1H), 1.00(d, J=6.5 Hz, 3H). MS(m/z): 626.3(M + H). |
| 171 | 61 | 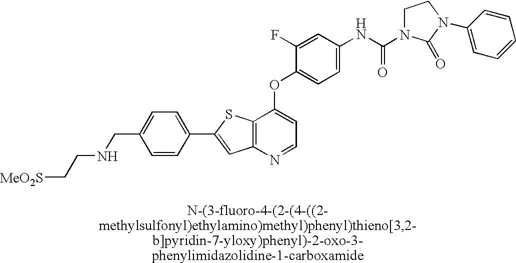 | ¹H NMR(400 MHz, DMSO-d₆) δ(ppm): 10.59(s, 1H), 8.51( d, J=5.2 Hz, 1H), 8.04(s, 1H), 7.89-7.84(m, 3H), 7.64(dt, J=8.0, 0.8 Hz, 2H), 7.54-7.40(m, 6H), 7.18(t, J=7.6 Hz, 1H), 6.64(d, J=5.2 Hz, 1H), 4.02- 3.92(m, 4H), 3.77(s, 2H), 3.27(t, J=6.8 Hz, 2H), 3.03(s, 3H), 2.92(t, J=6.8 Hz, 2H). MS(m/z): 660.3(M + H). |
| 172 | 62 | 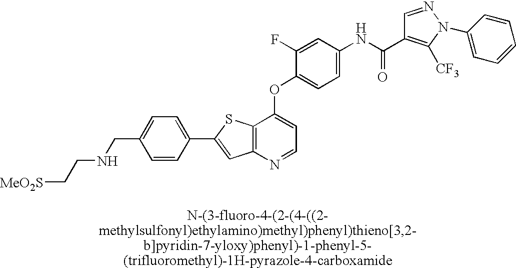 | ¹H NMR(400 MHz, DMSO-d₆) δ(ppm): 10.93(s, 1H), 8.52(d, J=5.6 Hz, 1H), 8.36(s, 1H), 8.20(s, 1H), 8.06(s, 1H), 8.97(d, J=12.4 Hz, 1H), 7.87(d, J=8.0 Hz, 2H), 7.67-7.52(m, 7H), 7.48(d, J=8.0 Hz, 2H), 6.67(d, J=5.6 Hz, 1H), 3.78(s, 2H), 3.28(t, J=6.4 Hz, 2H), 3.04(s, 3H), 2.92(t, J=6.4 Hz, 2H). MS(m/z): 710.1 (M + H). |
| 173 | 63 | 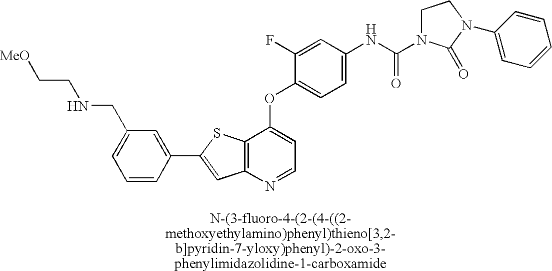 | ¹H NMR(400 MHz, DMSO-d₆) δ(ppm): 10.59(s, 1H), 8.53(d, J=5.6 Hz, 1H), 8.17(s, 1H), 8.06(s, 1H), 7.92(s, 1H), 7.87(dd, J=8.8, 2.4 Hz, 1H), 7.82(dt, J=7.2, 1.6 Hz, 1H), 7.64(d, J=8.0 Hz, 2H), 7.54-7.40(m, 6H), 7.18(t, J=7.2 Hz, 1H), 6.66(dd, J=5.6, 1.2 Hz, 1H), 4.0-3.94(m, 4H), 3.93(s, 2H), 3.47(t, J=5.6 Hz, 2H), 3.27(s, 3H), 2.81(t, J=5.6 Hz, 2H) (presumably formate salt). MS(m/z): 612.3(M + H). |
| 174 | 64 | 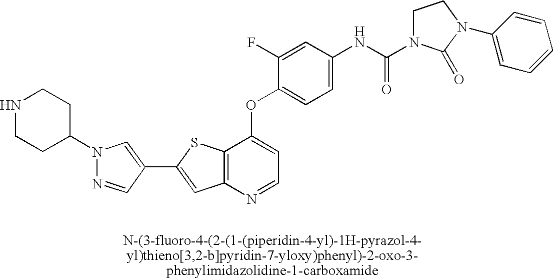 | ¹H NMR(400 MHz, DMSO-d₆) δ(ppm): 10.59(s, 1H), 8.82-8.70(m, 1H), 8.55-8.40(m, 3H), 8.10(s, 1H), 7.86(dd, J=12.8, 2.0 Hz, 1H), 7.76 (s, 1H), 7.64(dd, J=8.4, 1.0 Hz, 2H), 7.52-7.40(m, 4H), 7.19(t, J=7.2 Hz, 1H), 6.62(d, J=5.6 Hz, 1H), 4.62- 4.51(m, 1 H), 4.02-3.91(m, 4H), 3.44 (d, J=12.8 Hz, 2H), 3.11(q, J=11.8 Hz, 2H), 2.30- 2.09(m, 4H) (presumably formate salt). MS(m/z): 598.2(M + H). |
| 175 | 65 | 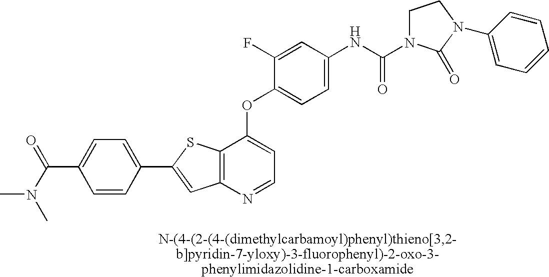 | ¹H NMR(400 MHz, DMSO-d₆) δ(ppm): 10.59(s, 1H), 8.54(d, J=5.6 Hz, 1H), 8.16(s, 1H), 7.96(d, J=8.8 Hz, 2H), 7.87(dd, J=13.2, 2.4 Hz, 1H), 7.64(d, J=8.0 Hz, 2H), 7.57- 7.40(m, 6H), 7.18(t, J=7.6 Hz, 1H), 6.68(dd, J=5.6, 0.8 Hz, 1H), 4.02- 3.91(m, 4H), 3.01(s, 3H), 2.96(s, 3H). MS(m/z): 596.1(M + H). |
| 176 | 66 | 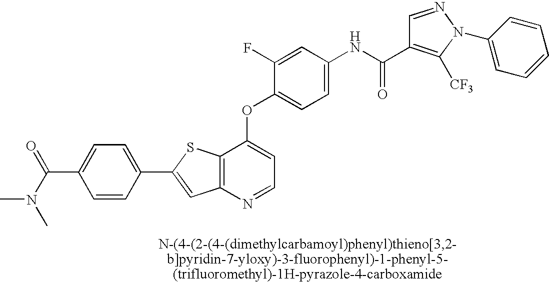 | ¹H NMR(400 MHz, DMSO-d₆) δ(ppm): 10.92(s, 1H), 8.54(d, J=5.6 Hz, 1H), 8.36(s, 1H), 8.17(s, 1H), 8.00-7.94(m, 3H), 7.65-7.53(m, 9H), 6.70(dd, J=5.6, 1.2 Hz, 1H), 3.01(s, 3H), 2.96(s, 3H). MS(m/z): 646.1 (M + H). |
| 177 | 67 | 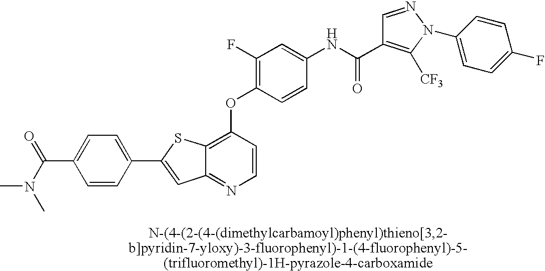 | ¹H NMR(400 MHz, DMSO-d₆) δ(ppm): 10.95(s, 1H), 8.54(d, J=5.2 Hz, 1H), 8.37(s, 1H), 8.17(s, 1H), 8.02-7.93(m, 3H), 7.69-7.52(m, 6H), 7.47(t,J8.8 Hz,2H), 6.70(d,J=5.2 Hz, 1H), 3.01(s, 3H), 2.96(s, 3H). MS(m/z): 664.0(M + H). |
| 178 | 68 | 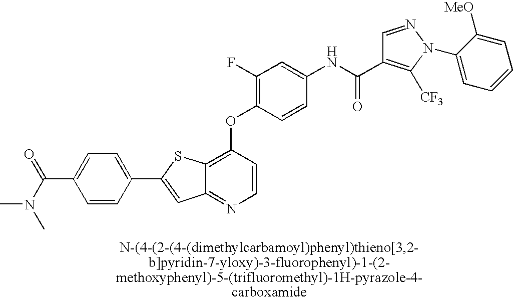 | ¹H NMR(400 MHz, DMSO-d₆) δ(ppm): 10.85(s, 1H), 8.54(d, J=5.6 Hz, 1H), 8.36(s, 1H), 8.17(s, 1H), 8.01-7.94(m, 3H), 7.65-7.52(m, 5H), 7.47(dd, J=8.0, 1.6 Hz, 1H), 7.30 (dd, J=8.4, 0.8 Hz, 1H), 7.14(td, J=7.6, 1.2 Hz, 1H), 6.70(d, J=5.6 Hz, 1H), 3.80(s, 3H), 3.01(s, 3H), 2.96(s, 3H). MS(m/z): 676.3(M + H). |
| 179 | 69 | 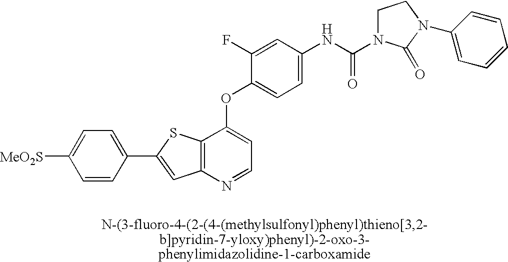 | ¹H NMR(400 MHz, DMSO-d₆) δ(ppm): 10.60(s, 1H), 8.57(d, J=5.6 Hz, 1H), 8.31(s, 1H), 8.20(d, J=8.4 Hz, 2H), 8.14(s, formate, 0.4H), 8.05 (d, J=8.4 Hz, 2H), 7.88(dd, J=13.2, 2.4 Hz, 1H), 7.64(d, J=7.6 Hz, 2H), 7.53(t, J=8.8 Hz, 1H), 7.50-7.40(m, 3H), 7.19(t, J=7.2 Hz, 1H), 6.71(d, J=4.4 Hz, 1H), 4.02-3.92(m, 4H), 3.29 (s, 3H)(presumably formate salt). MS (m/z): 603.1(M + H). |
| 180 | 70 | 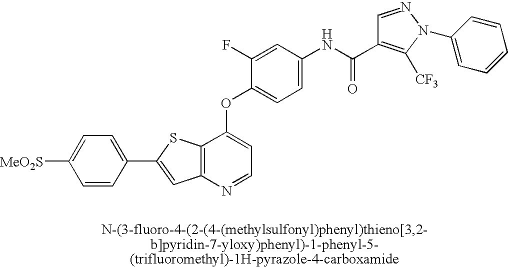 | ¹H NMR(400 MHz, DMSO-d₆) δ(ppm): 10.92(s, 1H), 8.58(d, J=5.6 Hz, 1H), 8.36(s, 1H), 8.32(s, 1H), 8.20(d, J 8.8Hz, 2H), 8.05(d, J=8.8 Hz, 2H), 7.97(dd, J=12.4, 2.0 Hz, 1H), 7.66-7.53(m, 7H), 6.74(dd, J=5.6, 0.8 Hz, 1H), 3.29(s, 3H). MS (m/z): 653.0(M + H). |
| 181 | 71 | 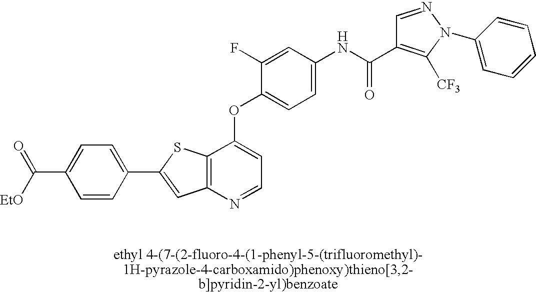 | ¹H NMR(400 MHz, DMSO-d₆) δ(ppm): 10.92(s, 1H), 8.56(d, J=5.6 Hz, 1H), 8.36(s, 1H), 8.26(s, 1H), 8.12-8.05(m, 4H), 7.97(d, J=12.0 Hz, 1H), 7.66-7.52(m, 7H), 6.72(d, J=5.6 Hz, 1H), 4.36(q, J=7.2 Hz, 2H), 1.35(t, J=7.2 Hz, 3H). MS (m/z): 647.1(M + H). |
| 182 | 72 | 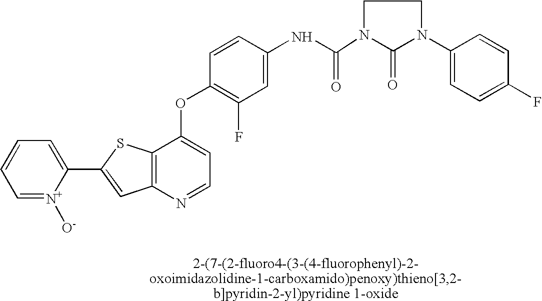 | ¹H NMR(400 MHz, DMSO-d₆) δ(ppm): 10.55(s, 1H), 8.69(s, 1H), 8.65(dd, J=8.2, 1.6 Hz, 1H), 8.59(d, J=5.7Hz, 1H), 8.49(dd, J 6.5,0.8 Hz, 1H), 7.85(dd, J=12.7, 2.4 Hz, 1H), 7.4-7.7(m, 6H), 7.28(m, 2H), 6.67(d, J=5.5 Hz, 1H), 3.95(s, 4H). MS(m/z): 560.2(M + 1). |
| 183 | 73 | 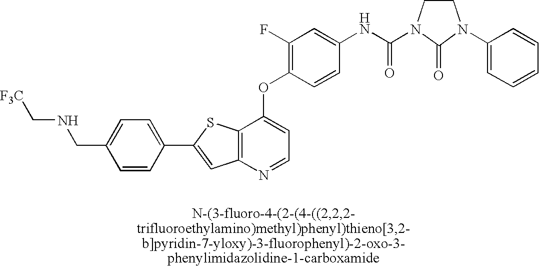 | ¹H NMR(400 MHz, DMSO-d₆) δ(ppm): 10.59(s, 1H), 8.51(d, J=5.5 Hz, 1H), 8.05(s, 1H), 7.88-7.84(m, 3H), 7.65-7.62(m, 2H), 7.49-7.41(m, 6H), 7.20-7.15(m, 1H), 6.64(dd, J=5.5, 0.8 Hz, 1H), 4.01-3.92(m, 4H), 3.84(s, 2H), 3.25-3.17(m, 2H), 3.08- 3.00(m, 1H). MS(m/z): 636.2 (M + H). |
| 184 | 74 | 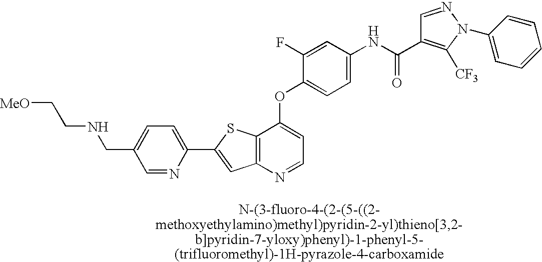 | ¹H NMR(400 MHz, DMSO-d₆) δ(ppm): 10.93(s, 1H), 8.58(d, J=2.2 Hz, 1H), 8.54(d, J=5.3 Hz, 1H), 8.37 (s, 1H), 8.34(s, 1H), 8.25(d, J=8.0 Hz, 1H), 8.18(s, 1H), 7.96(dd, J=12.7, 2.2 Hz, 1H), 7.91(dd, J=8.2, 2.2 Hz, 1H), 7.64-7.60(m, 3H), 7.59- 7.53(m, 4H), 6.71(dd, J=5.5, 1.0 Hz, 1H), 3.81(s, 2H), 3.42(t, J=5.7 Hz, 2H), 3.25(s, 3H), 2.68(t, J=5.5 Hz, 2H)(presumably formate salt). MS(m/z): 663.1(M + H). |
| 185 | 75 | 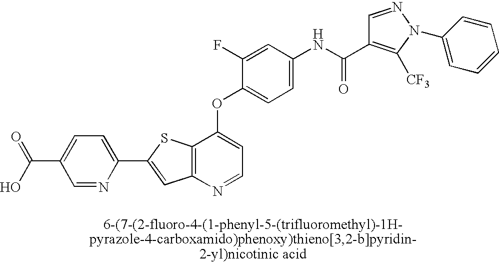 | ¹H NMR(400 MHz, DMSO-d₆) δ(ppm): 10.97(s, 1H), 9.04(t, J=1.4 Hz, 1H), 8.55(d, J=5.5 Hz, 1H), 8.43 (s, 1H), 8.37(s, 1H), 8.31(s, 2H), 7.97 (dd, J=12.9, 2.2 Hz, 1H), 7.62-7.53 (m, 7H), 6.73(d, J=5.5 Hz, 1H). MS (m/z): 620.1(M + H). |
| 186 | 76 | 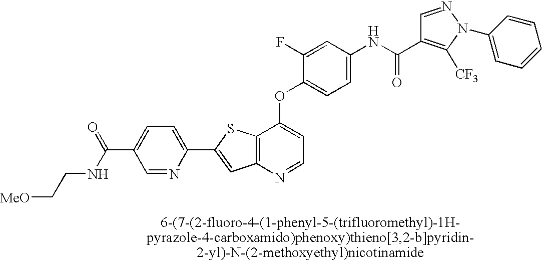 | ¹H NMR(400 MHz, DMSO-d₆) δ(ppm): 10.92(s, 1H), 9.04(s, 1H), 8.84-8.82(m, 1H), 8.56(d, J=5.5 Hz, 1H), 8.49(s, 1H), 8.41(d, J=8.2 Hz, 1H), 8.36-8.32(m, 2H), 7.96(dd, J=12.7, 1.6 Hz, 1H), 7.63-7.53(m, 7H), 6.74(d, J=5.3 Hz, 1H), 3.48-3.44(m, 4H), 3.28(s, 3H). MS(m/z): 677.1 (M + H). |
| 187 | 77 | 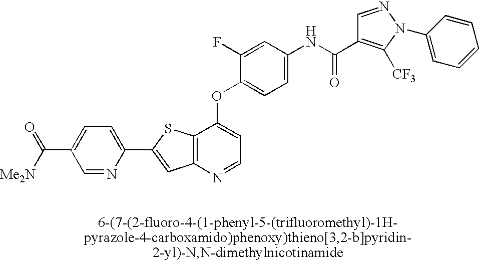 | ¹H NMR(400 MHz, DMSO-d₆) δ(ppm): 10.92(s, 1H), 8.69(d, J=1.2 Hz, 1H), 8.56(d, J=5.3 Hz, 1H), 8.47 (s, 1H), 8.37-8.34(m, 2H), 8.02(dd, J=8.2, 2.2 Hz, 1H), 7.96(dd, J=13.3, 2.2 Hz, 1H), 7.63-7.53(m, 7H), 6.74 (d, J=5.3 Hz, 1H), 3.02(s, 3H), 2.98 (s, 3H). MS(m/z): 647.5(M + H). |
| 188 | 78 | 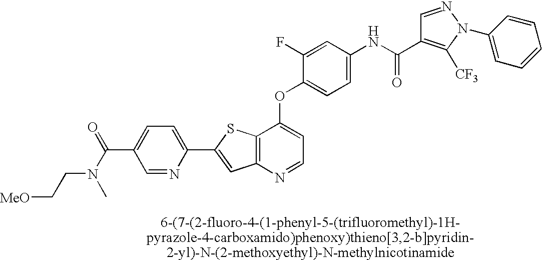 | ¹H NMR(400 MHz, DMSO-d₆) δ(ppm): 10.94(s, 1H), 8.66(s, 1H), 8.57(s, 1H), 8.47(s, 1H), 8.38-8.35 (m, 2H), 8.00-7.95(m, 2H), 7.65-7.52 (m, 7H), 6.76(s, 1H), 3.65-3.55(m, 2H), 3.45(s, 3H), 3.23(m, 2H), 3.03 (s, 3H). MS(m/z): 691.1(M + H). |
| 189 | 79 | 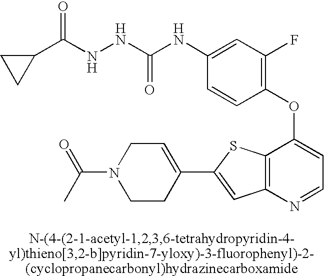 | ¹H-NMR(DMSO-d₆, 400 MHz): 9.89 (s, 1H), 9.32(br, 1H), 8.45(d, J=5.3 Hz, 1H), 8.35(s, 1H), 7.73-7.71(m, 1H), 7.57-7.55(m, 1H), 7.41-7.33(m, 2H), 6.12-6.59(m, 1H), 6.42-6.40(m, 1H), 4.22-4.15(m, 2H), 3.70-3.65(m, 2H), 2.69-2.60(m, 2H), 2.09(s, 1.7H), 2.05(s, 1.3H), 1.65-1.61(m, 1H), 0.71-0.72(m, 4H). MS(m/z): 510.2 (M + H). |
| 190 | 80 | 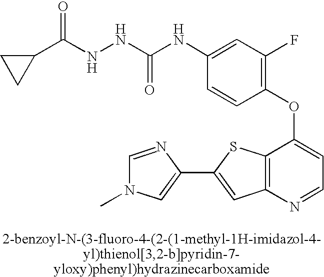 | ¹H-NMR(400 MHz, DMSO-d₆) δ(ppm): 10.34(br, 1H), 9.56(br, 1H), 8.69(br, 1H), 8.42(d, J=5.5 Hz, 1H), 7.94-7.92(m, 2H), 7.86(d, J=1.0 Hz, 1H), 7.77-7.74(m, 1H), 7.72(d, J=1.0 Hz, 1H), 7.68(s, 1H), 7.61-7.57 (m, 1H), 7.53-7.49(m, 2H), 7.42-7.35 (m, 2H), 6.56(d, J=4.9 Hz, 1H), 3.72 (s, 3H). MS(m/z): 503.2(M + H). |
| 191 | 81 | 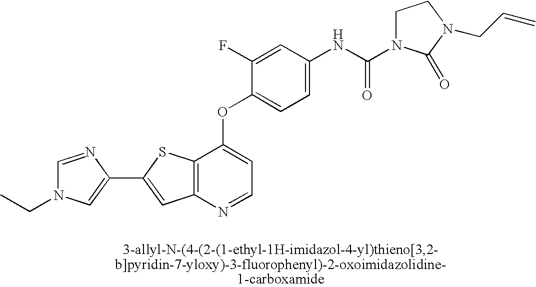 | ¹H-NMR(400 MHz, DMSO-d₆) δ(ppm): 10.60(s, 1H), 8.44(d, J=5.5 Hz, 1H), 7.96(s, 1H), 7.96-7.78(m, 2H), 7.68(s, 1H), 7.46(t, J=9.0 Hz, 1H), 7.36(dd, J=9.0, 1.6Hz, 1H), 6.58(d, J=5.5 Hz, 1H), 5.86-5.76(m, 1H), 5.30-5.21(m, 2H), 4.06(q, J=7.2 Hz, 2H), 3.86-3.8 1(m, 4H), 3.45- 3.77(m, 2H), 1.40(t, J=7.2 Hz, 3H). MS(m/z): 507.2(M + H). |
| 192 | 82 | 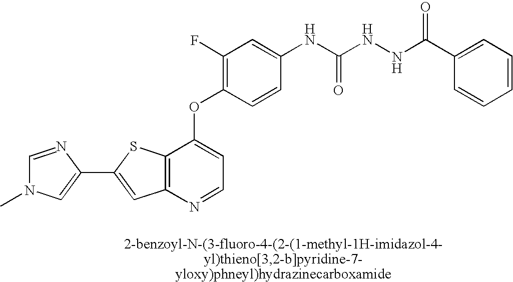 | ¹H-NMR(400 MHz, DMSO-d₆) 10.34 (br, 1H), 9.56(br, 1H), 8.69(br, 1H), 8.42(d, J=5.5 Hz, 1H), 7.94-7.92(m, 2H), 7.86(d, J 1.0 Hz, 1H), 7.77- 7.74(m, 1H), 7.72(d, J=1.0 Hz, 1H), 7.68(s, 1H), 7.61-7.57(m, 1H), 7.53- 7.49(m, 2H), 7.42-7.35(m, 2H), 6.56 (d, J=4.9 Hz, 1H), 3.72(s, 3H). MS (m/z): 503.2(M + 1). |
| 193 | 83 | 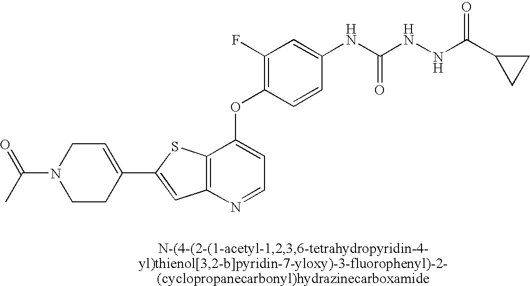 | ¹H-NMR(400 MHz, DMSO-d₆): 9.89 (s, 1H), 9.32(br, 1H), 8.45(d, J=5.3 Hz, 1H), 8.35(s, 1H), 7.73-7.71(m, 1H), 7.57-7.55(m, 1H), 7.41-7.33(m, 2H), 6.12-6.59(m, 1H), 6.42-6.40(m, 1H), 4.22-4.15(m, 2H), 3.70-3.65(m, 2H), 2.69-2.60(m, 2H), 2.09(s, 1.7H), 2.05(s, 1.3H), 1.65-1.61(m, 1H), 0.71-0.72(m, 4H). MS(m/z,): 510.2 (M + 1). |
| 194 | 84 | 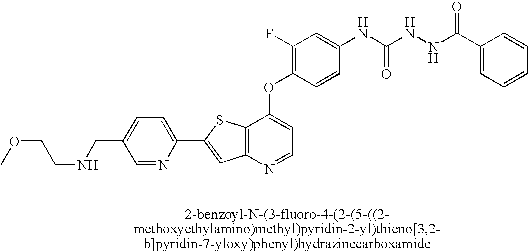 | ¹H-NMR(DMSO-D6, 400 MHz) 10.36(br, 1H), 9.37(br, 1H), 8.57- 8.49(m, 3H), 8.38(s, 1H), 8.23(dd, J8.2Hz, 1H), 7.95-7.88(m, 3H), 1 H), 7.52-7.49(m, 2H), 7.45-7.41(m, 2H), 6.66(d, J=5.5Hz, 1H), 3.78(s, 2H), 3.41(t, J=5.7Hz, 2H), 3.24(s, 3H), 2.65(t, J=5.7Hz, 2H), 2.28(br, 1H). m/z: 294.2(100%)(M + 1)⁺²/2, 587.3(48%)(M + 1)⁻. |
| 195 | 85 | 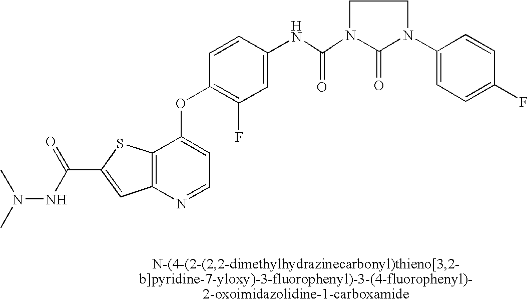 | DMSO-d6 10.55(s, 0.44H), 10.54(s, 0.56H), 9.94(s, 0.44H), 9.47(s, 0.56H), 8.57(dd, 1H, J5.5 Hz, J=7.8 Hz), 8.20(s, 1H), 7.84(dd, 1H, J=2.2 Hz, J=13.0 Hz), 7.64(m, 2H), 7.4- 7.55(m, 2H), 7.27(t, 2H, J=9.0 Hz), 6.76(d, 0.44H, J=5.1 Hz), 6.68(d, 0. 56H, J=5.5 Hz), 3.94(s, 4H), 2.62 (s, 2.7H), 2.60(s, 3.3H) mixture of 2 rotamers MS(m/z): (M + 1) 553.3(100%) |
| 196 | 86 | 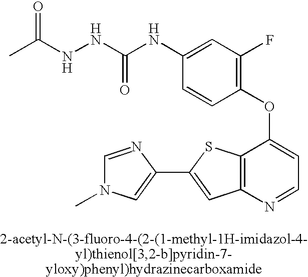 | MS(m/z): 441.2(M + H). |
| 197 | 87 | 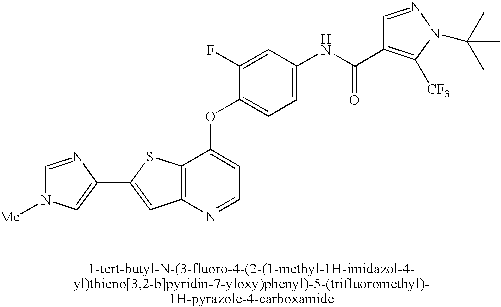 | ¹H NMR(400 MHz, DMSO-d₆) δ(ppm) :10.88(s, 1H), 8.43(d, J=5.5 Hz, 1H), 7.98(s, 1H), 7.94-7.85(m, 2H), 7.72(bs, 1H), 7.69(s, 1H), 7.56- 7.46(m, 2H), 6.60(d, J=5.5 Hz, 1H), 3.73(s, 3H), 1.67(s, 9H). MS(m/z): 559.2(M + H). |
| 198 | 88 | 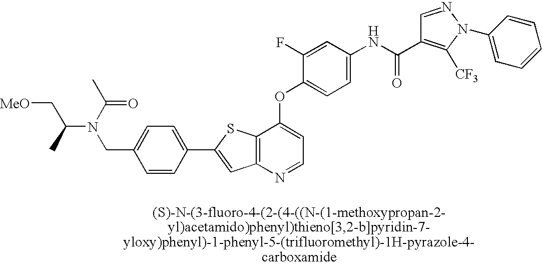 | MS(m/z): 718.3(M + H). |
| 199 | 89 | 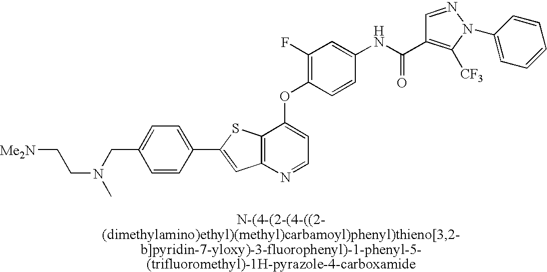 | MS(m/z): 703.3(M + H). |
| 200 | 90 | 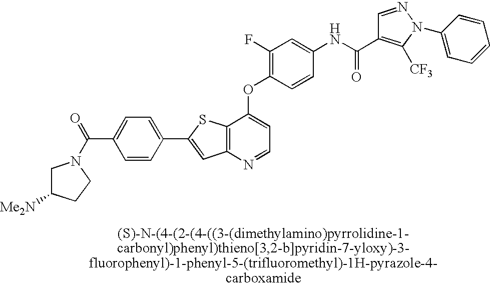 | MS(m/z): 715.3(M + H). |
| 201 | 91 | 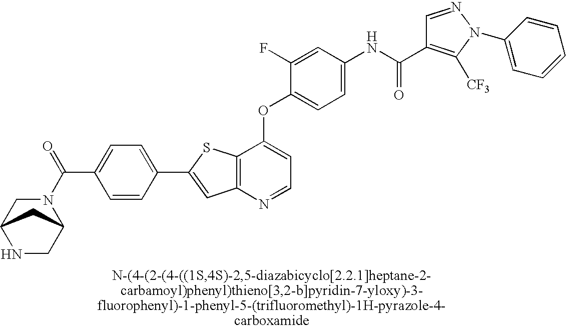 | MS(m/z): 699.3(M + H). |
| 202 | 92 | 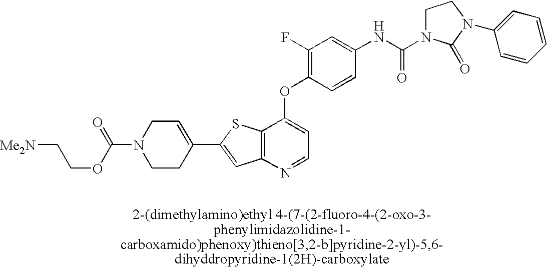 | ¹H NMR(400 MHz, DMSO-d₆) δ(ppm): 1H: 10.57(s, 1H), 8.47(d, J=5.5 Hz, 1H), 8.14(s, 0.5H), 7.83 (dd, J=2.3Hz, J=13.1 Hz, 1H), 7.62(m, 2H), 7.56(s, 1H), 7.48-7.4 1(m, 4H), 7.18(t, J=7.5 Hz, 1H), 6.26(d, J=5.5 Hz, 1H), 6.41(m, 1H), 4.26(t, J=4.9Hz, 2H), 4.13(m, 2H), 3.99-3.92 (m, 4H), 3.05(m, 2H), 2.63(m, 2H), 2.58(s, 6H) MS(m/z): 645.2(M + H). |
| 203 | 93 | 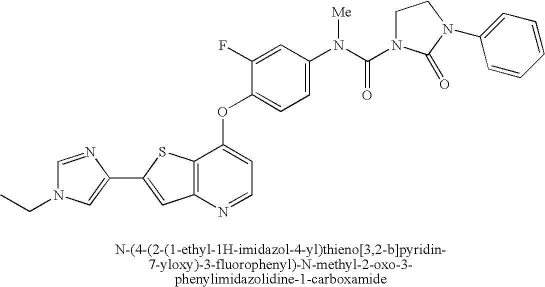 | ¹H NMR(400 MHz, DMSO-d₆) δ(ppm): 1H: 8.21(d, J=5.4Hz, 1H), 7.97(d, J=1.2Hz, 1H), 7.81(d, J=1.2Hz, 1H), 7.67(s, 1H), 7.61(dd, J=2.5Hz, J=12.1 Hz, 1H), 7.49-7.42 (m, 3H), 7.3 8-7.30(m, 3H), 7.12-7.08 (m, 1H), 6.31(dd, J=0.6 Hz, J=5.4 Hz, 1H), 4.06(q, J=7.2 Hz, 2H), 3.86(s, 4H), 3.38(s, 3H), 1.40(t, J=7.2 Hz, 3H). MS(m/z): 557.2(M + H). |
| 204 | 94 | 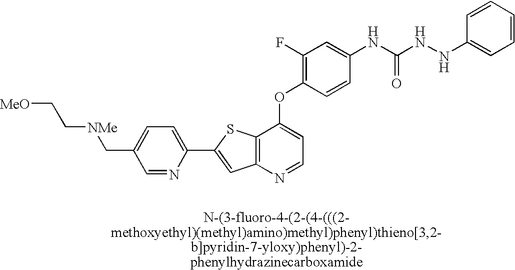 | ¹H NMR(400 MHz, DMSO-d₆) δ(ppm): 1H: 9.71(s, 1H), 8.48(d, J=5.47 Hz, 1H), 8.41(s, 1H), 8.13(s, 1H), 8.04(s, 1H), 7.85(m, 4H), 7.46 (m, 3H), 7.38(t, J=9.19Hz, 1H), 7.22(t, J=8.22 Hz, 1H), 6.78(m, 3H), 6.60(d, J=5.47 Hz, 1H), 3.62(s, 2H), 3.33(s, 3H), 3.24(s, 3H), 3.15 (m, 1H). MS(m/z): 572.56(M + H). |

### Pharmaceutical Compositions

In one embodiment, the invention provides pharmaceutical compositions comprising an inhibitor of VEGF receptor signaling and HGF receptor signaling according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In certain preferred embodiments, compositions of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt(s)” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

Inhibition of VEGF Receptor Signaling and HGF Receptor Signaling

In another embodiment the invention provides a method of inhibiting VEGF receptor signaling and HGF receptor signaling in a cell, comprising contacting a cell in which inhibition of VEGF receptor signaling and HGF receptor signaling is desired with an inhibitor of VEGF receptor signaling and HGF receptor signaling according to the invention. Because compounds of the invention inhibit VEGF receptor signaling and HGF receptor signaling, they are useful research tools for in vitro study of the role of VEGF receptor signaling and HGF receptor signaling in biological processes.

Preferably, the method according to this embodiment of the invention causes an inhibition of cell proliferation of the contacted cells. The phrase “inhibiting cell proliferation” is used to denote an ability of an inhibitor of VEGF receptor signaling and HGF receptor signaling to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers and comparing the size of the growth of contacted cells with non-contacted cells.

Preferably, growth of cells contacted with the inhibitor is retarded by at least 50% as compared to growth of non-contacted cells. More preferably, cell proliferation is inhibited by 100% (i.e., the contacted cells do not increase in number). Most preferably, the phrase “inhibiting cell proliferation” includes a reduction in the number or size of contacted cells, as compared to non-contacted cells. Thus, an inhibitor of VEGF receptor signaling and HGF receptor signaling according to the invention that inhibits cell proliferation in a contacted cell may induce the contacted cell to undergo growth retardation, to undergo growth arrest, to undergo programmed cell death (i.e., to apoptose), or to undergo necrotic cell death.

In some preferred embodiments, the contacted cell is a neoplastic cell. The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some preferred embodiments, the contacted cell is in an animal. Thus, the invention provides a method for treating a cell proliferative disease or condition in an animal, comprising administering to an animal in need of such treatment a therapeutically effective amount of a VEGF receptor signaling and HGF receptor signaling inhibitor of the invention. Preferably, the animal is a mammal, more preferably a domesticated mammal. Most preferably, the animal is a human.

The term “cell proliferative disease or condition” is meant to refer to any condition characterized by aberrant cell growth, preferably abnormally increased cellular proliferation. Examples of such cell proliferative diseases or conditions amenable to inhibition and treatment include, but are not limited to, cancer. Examples of particular types of cancer include, but are not limited to, breast cancer, lung cancer, colon cancer, rectal cancer, bladder cancer, leukemia and renal cancer. In particularly preferred embodiments, the invention provides a method for inhibiting neoplastic cell proliferation in an animal comprising administering to an animal having at least one neoplastic cell present in its body a therapeutically effective amount of a VEGF receptor signaling and HGF receptor signaling inhibitor of the invention.

### Assay Examples

### Assay Example 1

Inhibition of c-met and VEGF Activity

The following protocols were used to assay the compounds of the invention.

In Vitro Receptor Tyrosine Kinase Assays (c-Met/HGF Receptor and VEGF Receptor KDR)

These tests measure the ability of compounds to inhibit the enzymatic activity of recombinant human c-Met/HGF receptor and VEGF receptor enzymatic activity.

A 1.3-kb cDNA corresponding to the intracellular domain of c-Met or c-Met IC (Genbank accession number NP000236-1 amino acid 1078 to 1337) is cloned into the BamHI/XhoI sites of the pBlueBacHis2A vector (Invitrogen) for the production of a histidine-tagged version of that enzyme. This construct is used to generate recombinant baculovirus using the Bac-N-Blue system according to the manufacturer's instructions (Invitrogen).

The c-Met IC protein is expressed in Hi-5 cells (Trichoplusia Ni) upon infection with recombinant baculovirus construct. Briefly, Hi-5 cells grown in suspension and maintained in serum-free medium (Sf900 II supplemented with gentamycin) at a cell density of about 2×10⁶ cells/ml are infected with the above-mentioned viruses at a multiplicity of infection (MOI) of 0.2 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Hi-5 cell pellets infected with the C-Met IC recombinant baculovirus are thawed and gently resuspended in Buffer A (20 mM Tris pH 8.0, 10% glycerol, 1 μg/ml pepstatin, 2 μg/ml Aprotinin and leupeptin, 50 μg/ml PMSF, 50 μg/ml TLCK and 10 μM E64, 0.5 mM DTT and 1 mM Levamisole) using 3 ml of buffer per gram of cells. The suspension is Dounce homogenized after which it is centrifuged at 22500 g, 30 min., 4° C. The supernatant (cell extract) is used as starting material for purification of c-Met IC.

The supernatant is loaded onto a QsepharoseFF column (Amersham Biosciences) equilibrated with Buffer B (20 mM Tris pH 8.0, 10% glycerol) supplemented with 0.05M NaCl. Following a ten column volume (CV) wash with equilibration buffer, bound proteins are eluted with a 5 CV salt linear gradient spanning from 0.05 to 1M NaCl in Buffer B. Typically, the conductivity of selected fractions rank between 6.5 and 37 mS/cm. This Qsepharose eluate has an estimated NaCl concentration of 0.33M and is supplemented with a 5M NaCl solution in order to increase NaCl concentration at 0.5M and also with a 5M Imidazole (pH 8.0) solution to achieve a final imidazole concentration of 15 mM. This material is loaded onto a HisTrap affinity column (GE Healthcare) equilibrated with Buffer C (50 mM NaPO₄ pH 8.0, 0.5M NaCl, 10% glycerol) supplemented with 15 mM imidazole. After a 10 CV wash with equilibration buffer and an 8 CV wash with buffer C+40 mM imidazole, bound proteins are eluted with an 8 CV linear gradient (15 to 500 mM) of imidazole in buffer C. C-Met IC enriched fractions from this chromatography step are pooled based on SDS-PAGE analysis. This pool of enzyme undergoes buffer exchange using PD-10 column (GE Healthcare) against buffer D (25 mM HEPES pH 7.5, 0.1M NaCl, 10% glycerol and 2 mM β-mercaptoethanol). Final C-Met IC protein preparations concentrations are about 0.5 mg/ml with purity approximating 80%. Purified c-Met IC protein stocks are supplemented with BSA at 1 mg/ml, aliquoted and frozen at −80° C. prior to use in enzymatic assay.

In the case of VEGF receptor KDR a 1.6-kb cDNA corresponding to the catalytic domain of VEGFR2 or KDR (Genbank accession number AF035121 amino acid 806 to 1356) is cloned into the Pst I site of the pDEST20 Gateway vector (Invitrogen) for the production of a GST-tagged version of that enzyme. This construct is used to generate recombinant baculovirus using the Bac-to-Bac™ system according to the manucfacturer's instructions (Invitrogen).

The GST-VEGFR2₈₀₆₋₁₃₅₆ protein is expressed in Sf9 cells (Spodoptera frugiperda) upon infection with recombinant baculovirus construct. Briefly, Sf9 cells grown in suspension and maintained in serum-free medium (Sf900 II supplemented with gentamycin) at a cell density of about 2×10⁶ cells/ml are infected with the above-mentioned viruses at a multiplicity of infection (MOI) of 0.1 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Sf9 cell pellets infected with the GST-VEGFR2₈₀₆₋₁₃₅₆ recombinant baculovirus are thawed and gently resuspended in Buffer A (PBS pH 7.3 supplemented with 1 μg/ml pepstatin, 2 μg/ml Aprotinin and leupeptin, 50 μg/ml PMSF, 50 μg/ml TLCK and 10 μM E64 and 0.5 mM DTT) using 3 ml of buffer per gram of cells. Suspension is Dounce homogenized and 1% Triton X-100 is added to the homogenate after which it is centrifuged at 22500 g, 30 min., 4° C. The supernatant (cell extract) is used as starting material for purification of GST-VEGFR2₈₀₆₋₁₃₅₆.

The supernatant is loaded onto a GST-agarose column (Sigma) equilibrated with PBS pH 7.3. Following a four column volume (CV) wash with PBS pH 7.3+1% Triton X-100 and 4 CV wash with buffer B (50 mM Tris pH 8.0, 20% glycerol and 100 mM NaCl), bound proteins are step eluted with 5 CV of buffer B supplemented with 5 mM DTT and 15 mM glutathion. GST-VEGFR2₈₀₆₋₁₃₅₆ enriched fractions from this chromatography step are pooled based on U.V. trace i.e. fractions with high O.D.₂₈₀. Final GST-VEGFR2₈₀₆₋₁₃₅₆ protein preparations concentrations are about 0.7 mg/ml with purity approximating 70%. Purified GST-VEGFR2₈₀₆₋₁₃₅₆ protein stocks are aliquoted and frozen at −80° C. prior to use in enzymatic assay.

Inhibition of c-Met/HGF receptor and VEGFR/KDR is measured in a DELFIAT assay (Perkin Elmer). The substrate poly(Glu₄,Tyr) is immobilized onto black high-binding polystyrene 96-well plates. The coated plates are washed and stored at 4° C. During the assay, enzymes are pre-incubated with inhibitor and Mg-ATP on ice in polypropylene 96-well plates for 4 minutes, and then transferred to the coated plates. The subsequent kinase reaction takes place at 30° C. for 10-30 minutes. ATP concentrations in the assay are 10 uM for C-Met (5× the Kₘ) and 0.6 uM for VEGFR/KDR (2× the Kₘ). Enzyme concentration is 25 nM (C-Met) or 5 nM (VEGFR/KDR). After incubation, the kinase reactions are quenched with EDTA and the plates are washed. Phosphorylated product is detected by incubation with Europium-labeled anti-phosphotyrosine MoAb. After washing the plates, bound MoAb is detected by time-resolved fluorescence in a Gemini SpectraMax reader (Molecular Devices). Compounds are evaluated over a range of concentrations and IC₅₀'s (concentration of compounds giving 50% inhibition of enzymatic activity) are determined.

C-Met Phosphorylation Cell-Based Assay

This test measures the ability of compounds to inhibit HGF stimulated auto-phosphorylation of the c-Met/HGF receptor itself in a whole cell system.

MNNGHOS cell line expressing TPR-MET fusion protein are purchased from ATCC. The TPR-MET is the product of a chromosomal translocation placing the TPR locus on chromosome 1 upstream of the MET gene on chromosome 7 encoding for its cytoplasmic region catalytic domain. Dimerization of the Mᵣ 65,000 TPR-Met oncoprotein through a leucine zipper motif encoded by the TPR portion leads to constitutive activation of the met kinase. Constitutive autophosphorylation occurs on residues Tyr361/365/366 of TPR-Met. These residues are homologous to Tyr1230/1234/1235 of MET which become phosphorylated upon dimerization of the receptor upon HGF binding.

Inhibitor of c-Met formulated as 30 mM stocks in DMSO. For MNNGHOS treatments, cells, compounds are added to tissue culture media at indicated doses for 3 hours prior to cell lysis. Cells are lysed in ice-cold lysis buffer containing 50 mM HEPES (pH 7.5), 150 mM NaCl, 1.5 mM MgCl₂, 10% glycerol, 1% Triton X-100, 1 mM 4-(2-Aminoethyl)benzenesulfonyl fluoride hydrochloride, 200 μM sodium orthovanadate, 1 mM sodium fluoride, 10 μg/ml of leupeptin, 10 μg/ml of aprotinin/ml, 1 ug/ml of pepstatin and 50 ug/ml Na-p-Tosyl-L-lysine chloromethyl ketone hydrochloride.

Lysate are separated on 5-20% PAGE-SDS and immunoblots are performed using Immobilon P polyvinylidene difluoride membranes (Amersham) according to the manufacturer's instructions for handling. The blots are washed in Tris-buffered saline with 0.1% Tween 20 detergent (TBST). Tyr361/365/366 of TPR-Met are detected with polyclonal rabbit antibodies against tyrosine phosphorylated Met (Biosource International) and secondary antibodies anti-rabbit-horseradish peroxidase (Sigma) by chemiluminescence assays (Amersham, ECL) performed according to the manufacturer's instructions and followed by film exposure. Signal is quantitated by densitometry on Alpha-Imager. IC₅₀ values, as shown in Table 5, are defined as the dose required to obtain 50% inhibition of the maximal HGF stimulated phosphorylated c-Met levels.

The activities of some of the compounds according to the invention measured by the above assays are displayed in the following table. In the table (Table 5), “a” indicates inhibitory activity at a concentration of less than 250 nanomolar; “b” indicates inhibitory activity at a concentration ≧250 but <500 nanomolar, “c” indicates inhibitory activity at ≧500 but <1000 nanomolar; “d” indicates inhibitory activity ≧1000 nanomolar, and “e” indicates no activity as measured by that assay.

TABLE 5
Biological profile of selected compounds

| | | | | Y1230-34-35 |
|---|---|---|---|---|
| | | C-Met | VEGFR | TPR-MET phosphorylation |
| Example | Cpd | (IC₅₀, μM) | (IC₅₀, μM) | (IC₅₀, μM) |
| 1 | 8 | c | a | d |
| 2 | 12 | a | a | c |

Alternatively, the following assay is used to determine inhibition of c-Met phosphorylation.

The c-Met receptor is expressed in numerous cancer cell lines derived from tumors of epithelial origin. In MKN45 gastric carcinoma cells the c-Met gene is amplified, resulting in several-fold overexpression of the receptor and its constitutive activation. A sensitive method to follow c-Met phosphorylation in these cells was developed. In previous studies with earlier generation c-Met inhibitors, we established that the IC₅₀s for the inhibition of c-Met phosphorylation were identical using this novel ELISA approach and standard western blot procedure, with antibodies directed against the activating autocatalysis tyrosine residues of c-MET (Tyr Y1230-34-35).

Cell treatments: MKN45 cells are seeded into wells of 96-well plates at a density of 3×10⁴ cells/well in RPMI medium supplemented with 10% FBS. Cells are grown for 48 h prior to treatments with compounds of interest. Inhibitors are added to the medium in triplicate wells at the indicated doses. After 3 h of treatment, media is aspirated and cells are lysed by one freeze-thaw cycle in 50 μL/well hypotonic lysis buffer (25 mM HEPES pH 7.5, 10 mM NaCl with 1 mM 4-(2-aminoethyl)benzenesulfonyl fluoride hydrochloride, 200 μM sodium orthovanadate, 1 mM sodium fluoride, 10 μg/mL of leupeptin, 10 μg/mL of aprotinin/mL, 1 μg/mL of pepstatin and 50 μg/mL Na-p-tosyl-L-lysine chloromethyl ketone hydrochloride.

Detection of phosphorylated c-Met by direct ELISA: Lysate samples (5 μL) from wells of treatment plates are transferred to 80 μL of binding buffer (25 mM HEPES pH 7.5, 200 mM NaCl) in wells of high binding white polysterene 96-well plates (Corning). After an overnight protein binding incubation at 4° C., lysates are aspirated and wells are blocked for 1 h at 37° C. in TBST supplemented with 5% BSA. Plates are incubated with the primary antibodies anti-phospho-Tyrosine (Millipore, 4G10) diluted 1/15000 in TBST supplemented with 5% BSA for 1 h at room temperature. Plates are washed 3 times on a plate washer (SkanWasher, Molecular Devises), and incubated with the reporter antibody anti-rabbit-horseradish peroxidase (Sigma) diluted 1/15000 in TBST supplemented with 5% BSA, for 1 h at room temperature. Plates are washed 3 times with TBST using on a plate washer and subsequently incubated with chemiluminescent substrate solution (ECL, Roche). Luminescence signal is captured on a Polar Star Optima apparatus (BMG LabTech).

Average values of the triplicate samples are used to prepare IC₅₀ curves using a 4-parameter fit model. These curves are calculated using, for example, GraFit 5.0 software. For assay standardization purpose, an internal control is included on each experimental test plate.

The activities of some of the compounds according to the invention measured by the above assays are displayed in the following table (Table 6). In the table, “a” indicates inhibitory activity at a concentration of less than 250 nanomolar; “b” indicates inhibitory activity at a concentration ≧250 but <500 nanomolar, “c” indicates inhibitory activity at ≧500 but <1000 nanomolar; “d” indicates inhibitory activity ≧1000 nanomolar, and “e” indicates no activity as measured by that assay.

TABLE 6

| | | | | | |
|---|---|---|---|---|---|
| | | | | CMET | |
| | | | | | ELISA |
| | | | A549 | DU145 | UM = |
| | | | INHIB | SCATT | C-MET |
| | | | WND | HEAL- | IC50 in |
| | CMET | VEGFR | ING | IC50 | MKN45 |
| STRUCTURE | IC50 | IC50 | IC50 UM | UM | UM |

| | | | | | |
|---|---|---|---|---|---|
| 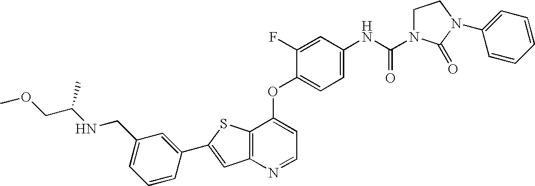 | a | a | b | b | a |
| 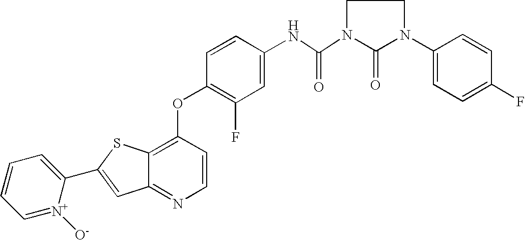 | a | a | a | a | a |
| 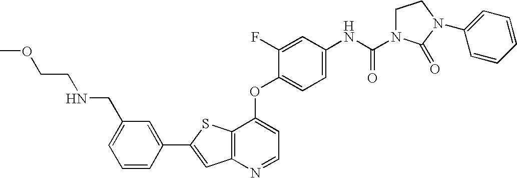 | a | a | b | a | a |
| 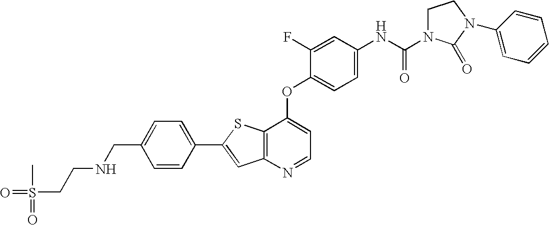 | a | a | d | b | a |
| 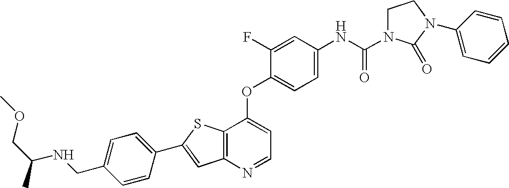 | a | a | a | a | a |
| 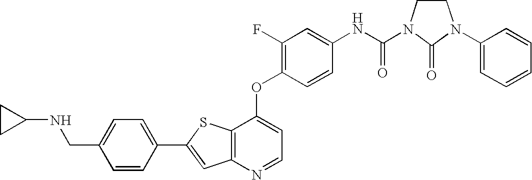 | a | a | b | a | a |
| 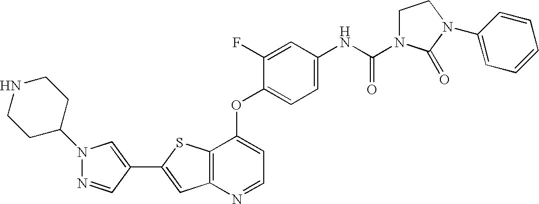 | a | a | d | b | a |
| 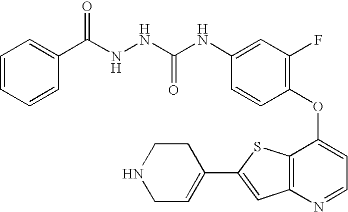 | b | a | d | d | d |
| 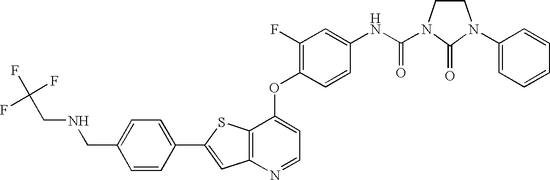 | | b | d | d | c |
| 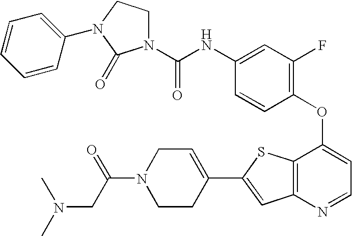 | a | a | a | a | a |
| 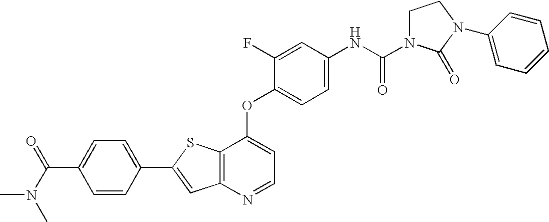 | a | a | a | b | a |
| 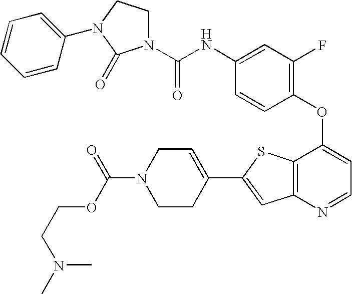 | a | a | b | b | a |
| 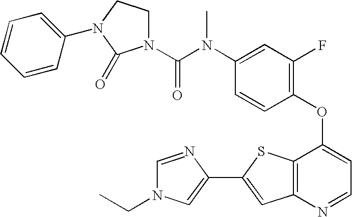 | c | a | d | d | c |
| 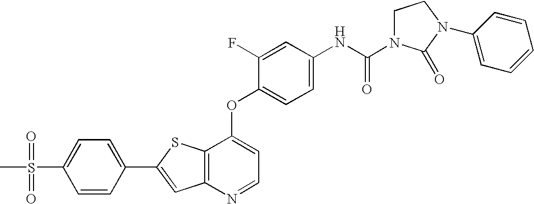 | b | b | | | a |
| 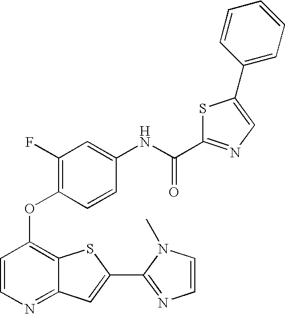 | d | d | | | |
| 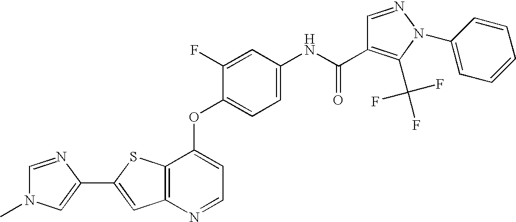 | a | d | b | d | c |
| 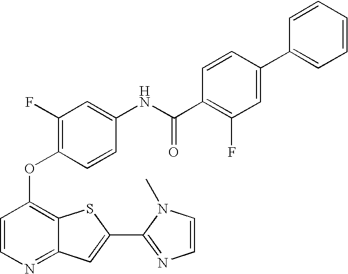 | a | d | | | |
| 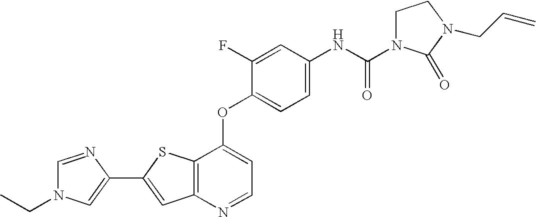 | a | a | b | b | b |
| 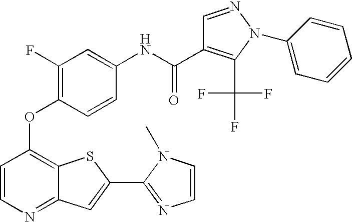 | a | d | d | d | |
| 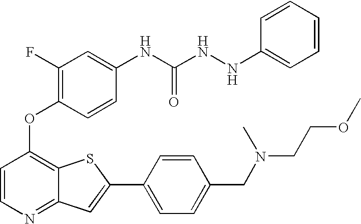 | b | d | b | d | a |
| 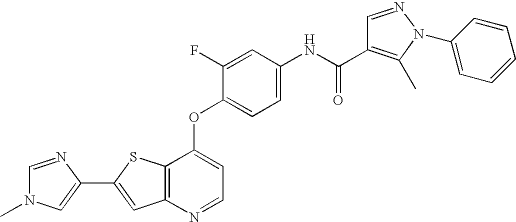 | a | d | b | d | d |
| 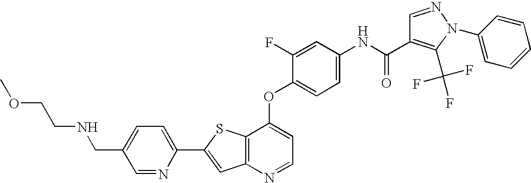 | a | d | b | b | b |
| 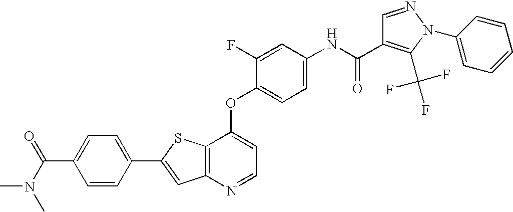 | a | d | a | b | a |
| 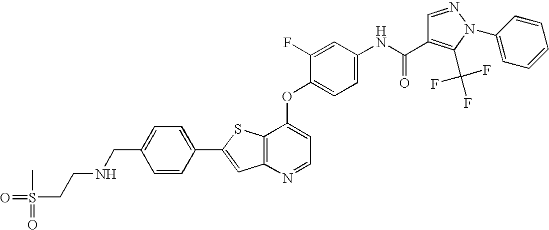 | a | d | d | d | d |
| 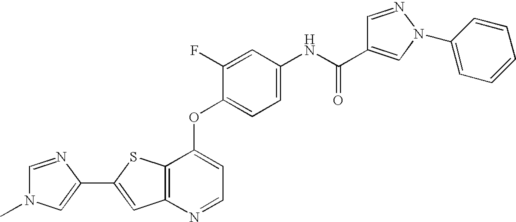 | a | d | b | d | c |
| 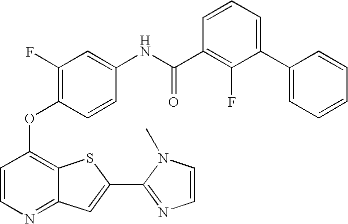 | a | a | b | d | c |
| 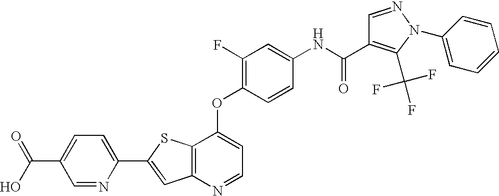 | a | d | d | e | d |
| 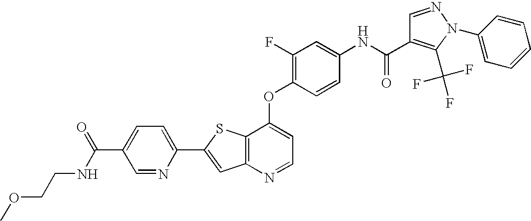 | a | d | e | e | e |
| 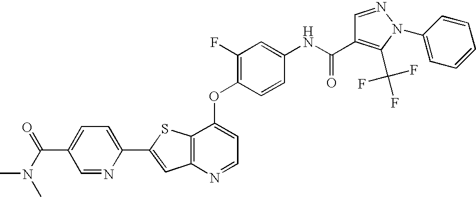 | a | d | b | b | |
| 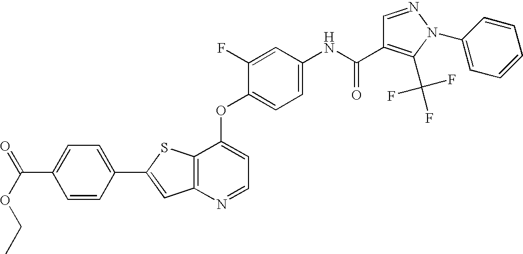 | d | d | d | d | |
| 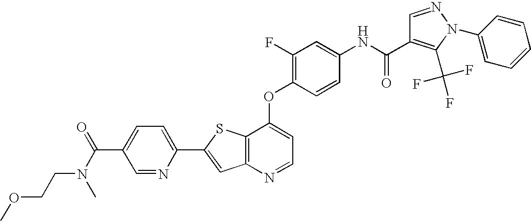 | a | d | b | d | |
| 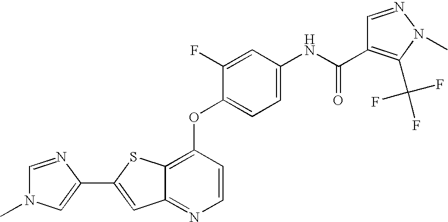 | a | c | d | e | |
| 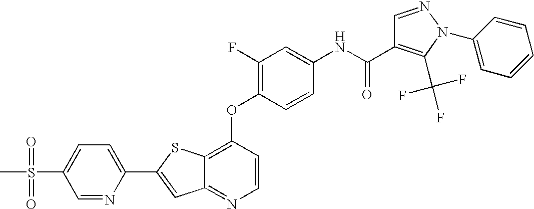 | a | d | a | b | |
| 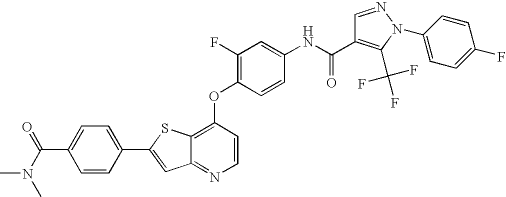 | a | d | d | d | |
| 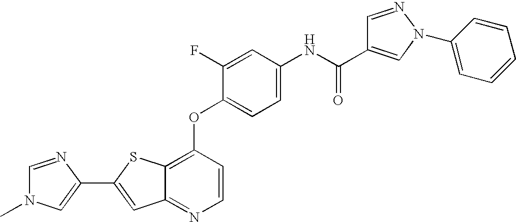 | a | c | d | d | |

In Vivo Solid Tumor Disease Model

This test measures the capacity of compounds to inhibit solid tumor growth.

Tumor xenografts are established in the flank of female athymic CD1 mice (Charles River Inc.), by subcutaneous injection of 1×10⁶ U87, A431 or SKLMS cells/mouse. Once established, tumors are then serially passaged s.c. in nude mice hosts. Tumor fragments from these host animals are used in subsequent compound evaluation experiments. For compound evaluation experiments female nude mice weighing approximately 20 g are implanted s.c. by surgical implantation with tumor fragments of 30 mg from donor tumors. When the tumors are approximately 100 mm³ in size (7-10 days following implantation), the animals are randomized and separated into treatment and control groups. Each group contains 6-8 tumor-bearing mice, each of which is ear-tagged and followed individually throughout the experiment.

Mice are weighed and tumor measurements are taken by calipers three times weekly, starting on Day 1. These tumor measurements are converted to tumor volume by the well-known formula (L+W/4)³ 4/3π. The experiment is terminated when the control tumors reach a size of approximately 1500 mm³. In this model, the change in mean tumor volume for a compound treated group/the change in mean tumor volume of the control group (non-treated or vehicle treated)×100 (ΔT/ΔC) is subtracted from 100 to give the percent tumor growth inhibition (% TGI) for each test compound. In addition to tumor volumes, body weight of animals is monitored twice weekly for up to 3 weeks.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1.-10. (canceled)

11. A compound of the Formula (V) and racemic mixtures, diastereomers and enantiomers thereof: 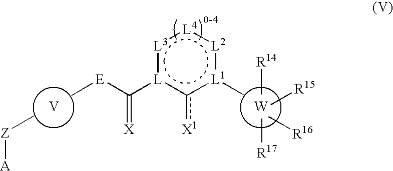
and N-oxides, hydrates, solvates, pharmaceutically acceptable salts, prodrugs and complexes thereof, wherein A, Z, V, E, X, W, R¹⁴, R¹⁵, R¹⁶ and R¹⁷ are as defined in claim 1; [symbol]
is a single or double bond;
X¹ is selected from the group consisting of O, S, CH₂, N—CN, N—O-alkyl, NH and N(C₁-C₆alkyl) when [symbol] is a double bond, or
X¹ is selected from the group consisting of H, halogen, alkyl, alkenyl, alkynyl, alkoxy, NH(alkyl) and alkyl-thio, each of which is optionally substituted, when [symbol] is a single bond;
L and L are independently selected from the group consisting of —CH—, —N—, —C(halogen)- and —C(C₁-C₆alkyl)-;
L² and L³ are independently selected from the group consisting of CH, CH₂, N, O and S;
L⁴ is selected from the group consisting of absent, CH, CH₂, N, O and S; and the group 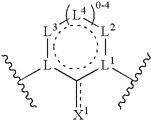
is aromatic or non-aromatic, provided that two 0 are not adjacent to each other;
with the proviso that when [symbol] is a single bond, Formula (V) excludes those compounds wherein
Z is O;
V is a 6 membered aryl ring system or a 6 membered heteroaryl ring system containing one heteroatom; and
X¹ is selected from the group consisting of H, halogen, alkyl, alkenyl, alkynyl, CN and alkoxy;
provided that this proviso does not exclude those compounds wherein W is substituted by either an alkenyl or alkynyl.
with the proviso that Formula (V) excludes those compounds wherein
Z is selected from the group consisting of O, S, CH₂, N(Bn), N(H) and N (optionally substituted alkyl);
E is N(H) or N(alkyl);
X is O;
D is selected from the group consisting of H, halogen, NO₂, cyano, OR^{b}, NR^{b}R^{b}, CO₂R^{b}, C(O)NR^{b}R^{b}, SO₂R^{b}, SO₂NR^{b}R^{b}, NR^{b}SO₂R^{b}, NR^{b}C(O)R^{b}, NR^{b}CO₂R^{b}, —CO(CH₂)₁R^{b}, —CONH(CH₂)₁R^{b}, alkylaminoalkyl, alklaminoalkynyl, C₁-C₆alkyl, substituted C₁-C₆alkyl, C₃-C₇cycloalkyl, substituted C₃-C₇ cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl and substituted heterocycloalkyl;
wherein R^{b} is selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl and substituted heterocycloalkyl; and
the group 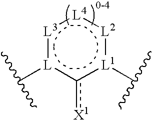
is selected from the group consisting of aryl, heteroaryl, and heterocylcoalkyl; and
with the proviso that Formula (V) excludes those compounds wherein
A is selected from the group consisting of 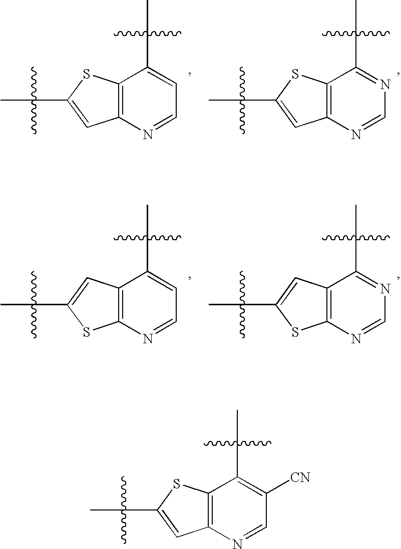
E is selected from the group consisting of —N(H)—, —N(C₁-C₆alkyl)- and —N(H)CH₂—;
R¹⁴, R¹⁵, R¹⁶ and R¹⁷ are each H;
Z is selected from the group consisting of —O—, —N(C(O)(C₁-C₆alkyl)), —S—, —CH₂—, —N(H)—, and —N(C₁-C₆alkyl); and
D is selected from the group consisting of —H, C₁-C₆ alkyl, C₃-C₁₀ cycloalkyl, —C(O)N42R⁴³, —Y—NR⁴²R⁴³, —NR⁴²C(═O)R⁴³, —SO₂R⁴², —SO₂NR⁴²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, C(═NR⁴²)NR³⁷R⁴³, —NR³⁷C(═NR⁴²)NR³⁷R⁴³, —C(O)R⁴², —CO₂R⁴², —C(O)(C₆-C₁₀ aryl), —Y—(C₆-C₁₀ aryl), —Y-(5-10 membered heterocyclyl), —CO₂R^{6a}, wherein the aforementioned D groups other than —H are optionally substituted, or is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different;
wherein R⁴², R⁴³, Y, R³⁷, R^{6a}, Z³ and Z⁴ are as defined in Formula (I); and
with the proviso that Formula (V) excludes those compounds wherein
X is O (except when Z¹² is OC(S) or C(O)) wherein Z¹² is as defined in Formula (I);
XI is O;
V is an unsubstituted 5 or 6 membered aryl ring system or an unsubstituted 5 or 6 membered heteroaryl ring system containing between one and three heteroatoms;
Z is selected from the group consisting of O, S, NH and N (optionally substituted C₁-C₄alkyl); and
D is selected from the group consisting of —H, halogen, trihalomethyl, —CN, nitro, —OR^{e}, —N(R^{e})R^{e}, —S(O)O₂R^{e}, —SO₂N(R^{e})R^{e}, —CO₂R^{e}, —C(O)N(R^{e})R^{e}, —N(R^{e})SO₂R^{e}, N(R^{e})C(O)R^{e}, —NCO₂R^{e}, —C(O)R^{e}, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₂-C₆alkenyl, optionally substituted aryl C₂-C₆alkynyl, optionally substituted heterocycle, optionally substituted heterocycle C₁-C₆alkyl, optionally substituted heterocyle C₂-C₆alkenyl, optionally substituted heterocycle C₂-C₆alkynyl, optionally substituted heteroaryl, optionally substituted heteroaryl C₁-C₆alkyl, optionally substituted heteroaryl C₂-C₆alkenyl, optionally substituted heteroaryl C₂-C₆alkynyl, and M-R^{e} wherein
each R^{e} is independently selected from the group consisting of H, optionally substituted C₁-C₆alkyl, optionally substituted C₂-C₆alkenyl, optionally substituted C₂-C₆alkynyl, optionally substituted aryl, optionally substituted aryl C₁-C₆alkyl, optionally substituted aryl C₁-C₆alkenyl, optionally substituted aryl C₁-C₆alkynyl, optionally substituted heterocycle, optionally substituted heteorcycle C₁-C₆alkyl, optionally substituted heteorcycle C₁-C₆alkenyl, optionally substituted heteorcycle C₁-C₆alkynyl; or any two of R^{e}, when taken together with a common nitrogen to which they are attached, can form an optionally substituted 5-7 membered heterocycle or an optionally substituted 5-7 membered heteroaryl, said optionally substituted 5-7 membered heterocycle or optionally substituted 5-7 membered heteroaryl optionally containing at least one additional annular heteroatom selected from N, O, S and P;
M is selected from the group consisting of —O—, —S(O)₀₋₂—, NH and N (optionally substituted C₁-C₆alkyl); and
R⁸⁰ is selected from the group consisting of H, halogen, —OR^{e}, —S(O)₀₋₂R^{e}, NO₂, —N(R^{c})R^{c}, and optionally substituted C₁-C₆alkyl;
provided that this proviso does not exclude those compounds wherein W is substituted by a halogen and either an alkenyl or alkynyl.

12. A compound represented by the Formula (V-A) and racemic mixtures, diastereomers and enantiomers thereof: 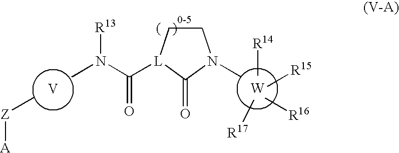
and N-oxides, hydrates, solvates, pharmaceutically acceptable salts, prodrugs and complexes thereof, wherein the groups A, Z, V, R¹⁴, R¹⁵, R¹⁶, R¹⁷ and W are as defined in claim 1; and wherein
L is either —CH— or N, and
R¹³ is selected from the group consisting of H, C₁-C₆alkyl, substituted C₁-C₆alkyl, cycloalkyl, substituted cycloalkyl, OH, unsubstituted —O—(C₁-C₆alkyl), substituted —O—(C₁-C₆alkyl).

13. The compound according to claim 12, wherein W is phenyl.

14.-35. (canceled)

36. A compound having the Formula (V-B) and racemic mixtures, diastereomers and enantiomers thereof: 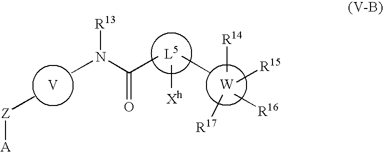
and N-oxides, hydrates, solvates, pharmaceutically acceptable salts, prodrugs and complexes thereof, wherein A, Z, V, W, R¹³, R¹⁴, R¹⁵, R¹⁶, R¹⁷ are as defined in Formula (I), and wherein L⁵ is selected from the group consisting of thiazolyl, phenyl and pyrazole, preferably pyrazole, and X^{h} is selected from the group consisting of absent, H, halogen, —NH₂, alkyl and —CF₃, preferably —CF₃

37. A pharmaceutical composition comprising a compound according to claim 11, and a pharmaceutically acceptable carrier.

38. A method of inhibiting kinase activity, the method comprising contacting the kinase with an inhibiting effective amount of a compound according to claim 11, or a composition thereof.

39. A method of inhibiting kinase activity in a cell, the method comprising contacting the cell with an inhibiting effective amount of a compound according to claim 11, or a composition thereof.

40. A method of treating a cell proliferative disease in a patient, the method comprising administering to the patient in need of such treatment a therapeutically effective amount of a compound according to claim 11, or a composition thereof.

41. A pharmaceutical composition comprising a compound according to claim 12, and a pharmaceutically acceptable carrier.

42. A method of inhibiting kinase activity, the method comprising contacting the kinase with an inhibiting effective amount of a compound according to claim 12, or a composition thereof.

43. A method of inhibiting kinase activity in a cell, the method comprising contacting the cell with an inhibiting effective amount of a compound according to claim 12, or a composition thereof.

44. A method of treating a cell proliferative disease in a patient, the method comprising administering to the patient in need of such treatment a therapeutically effective amount of a compound according to claim 12, or a composition thereof.

45. A pharmaceutical composition comprising a compound according to claim 13, and a pharmaceutically acceptable carrier.

46. A method of inhibiting kinase activity, the method comprising contacting the kinase with an inhibiting effective amount of a compound according to claim 13, or a composition thereof.

47. A method of inhibiting kinase activity in a cell, the method comprising contacting the cell with an inhibiting effective amount of a compound according to claim 13, or a composition thereof.

48. A method of treating a cell proliferative disease in a patient, the method comprising administering to the patient in need of such treatment a therapeutically effective amount of a compound according to claim 13, or a composition thereof.

49. A pharmaceutical composition comprising a compound according to claim 36, and a pharmaceutically acceptable carrier.

50. A method of inhibiting kinase activity, the method comprising contacting the kinase with an inhibiting effective amount of a compound according to claim 36, or a composition thereof.

51. A method of inhibiting kinase activity in a cell, the method comprising contacting the cell with an inhibiting effective amount of a compound according to claim 36, or a composition thereof.

52. A method of treating a cell proliferative disease in a patient, the method comprising administering to the patient in need of such treatment a therapeutically effective amount of a compound according to claim 36, or a composition thereof.
